(19) United States
(12) Patent Application Publication
(10) Pub. No.: US 2025/0269050 A1
(43) Pub. Date: Aug. 28, 2025

(54) CYCLIC DICHALCOGENIDE ADC LINKER UNITS AND USES THEREOF

(71) Applicants: Oliver THORN-SESHOLD, MUNICH (DE); Jan FELBER, MUNICH (DE); Lukas ZEISEL, MUNICH (DE)

(72) Inventors: Oliver THORN-SESHOLD, MUNICH (DE); Jan FELBER, MUNICH (DE); Lukas ZEISEL, MUNICH (DE)

(21) Appl. No.: 18/587,313

(22) Filed: Feb. 26, 2024

(51) Int. Cl. A61K 47/68; A61K 45/06; A61P 35/00

(52) U.S. Cl. CPC A61K 47/68037; A61K 45/06; A61K 47/6849; A61K 47/6889; A61P 35/00

(57) ABSTRACT

The present application relates to a multicomponent immunoconjugate that is capable of targeting a cellular environment and releasing a molecular cargo in the presence of a reductant, and is thus suitable for treating, ameliorating or preventing a disorder selected from a neoplastic disorder, particularly cancer; atherosclerosis; an autoimmune disorder; an inflammatory disease; and a chronic inflammatory autoimmune disease. Precursors of the immunoconjugate are also disclosed.

### TECHNICAL FIELD

The present application relates to compounds having the formula (I), (II), (III), (IV), or (V). The compound having the formula (I) is a multicomponent immunoconjugate that is capable of targeting a cellular environment and releasing a molecular cargo in the presence of a reductant, and is thus suitable for treating, ameliorating or preventing a disorder selected from a neoplastic disorder, particularly cancer; atherosclerosis; an autoimmune disorder; an inflammatory disease; and a chronic inflammatory autoimmune disease. The compounds having the formula (II), (III), (IV), or (V) can be used to assemble the multicomponent immunoconjugate of the formula (I) according to methods known in the art. The invention further relates to a pharmaceutical composition comprising the same.

### PRIOR ART

Most therapeutic drugs contain highly bioactive molecules that influence native biological systems with high potency and may cause cytotoxic effects. Systemic, untargeted administration of these therapeutics leads to high a level of systemic side effects that drastically affect tolerability in patients and limit the safe use of highly active therapeutic modalities. Efficient and low side-effect therapeutics (e.g., targeted anti-cancer drugs) require targeting strategies. Thereby, the therapeutic effect may, e.g., be directed by monoclonal antibodies for specific antigens with, e.g., cancer-correlated upregulation, such as the anti-HER2 targeted antibody trastuzumab. These can be used to deliver potent molecular payloads through antibody-drug conjugates (ADCs) (also referred to as “immunoconjugates”) that are internalized into tumor cells at a high degree of specificity and result in low systemic drug exposure; and ultimately high efficacy in patients with low adverse effects (Sievers et al. Annu. Rev. Med. 2013, 64, 15). Promising advances have been achieved in recent years by designing highly tuned molecular architectures: Antibody-drug conjugates usually comprise an active chemical agent (a highly bioactive therapeutic payload, e.g., cytotoxic drugs such as the antimitotic agent monomethyl auristatin E (MMAE)) that is chemically attached the monoclonal antibody through a linker unit of flexible choice (Fu et al. Signal Transduction and Targeted Therapy 2022, 7, 93). The biological activity of the therapeutic payload (i.e., the therapeutically active compound) is usually masked when bound within the immunoconjugate. Its activity can be restored upon cleavage of the therapeutic payload from the macromolecular conjugate. Additionally, during this process a functional group crucial for its biological activity may be liberated by this cleavage (e.g., being a crucial functional group for binding the target protein).

A flexible linker unit usually connects the active therapeutic payload to the targeting unit. The flexible linker unit usually contains a self-immolative spacer unit (e.g., para-aminobenzyloxycarbonyl-PABC) (Carl et al. J. Med. Chem. 1981, 24, 479) that is directly linked to the therapeutic payload. This spacer unit can be further attached to a cleavable unit (e.g., a short peptide labile to enzymatic proteolysis) prone to be cleaved in a biological environment. Desirably, it is cleaved intracellularly; and more desirably, it is mostly cleaved in inflamed/tumorous environments. After irreversible cleavage of this cleavable unit, e.g., through enzymatic intracellular reactions, the therapeutic payload is usually liberated by a chemical fragmentation reaction cascade promoted by the spacer unit (de Groot et al. J. Org. Chem. 2001, 66, 26, 8815). The cleavable unit is usually connected via a warhead group to a targeting unit, that guides the fully functional immunoconjugate to a target cell in a pathological diseased state. Preferred targeting units include monoclonal antibodies; alternatively, the targeting unit may also be a primary, polyclonal and/or humanized antibody, antibody mimetic, nanobody, de novo immunoprotein or (macro) molecular targeting ligand. The warhead group (e.g., a maleimide attached to the cleavable unit via its N atom) is usually a reactive functional group that allows for efficient conjugation of the linker-payload to the targeting unit, e.g., by reaction with an appropriate amino acid side chain at the targeting unit (e.g., a free sulfhydryl group from an antibody's cysteine side chain can react with a maleimide warhead). Research in antibody-drug conjugate design has primarily focused on choosing and tuning the biological target, the targeting unit, and the conjugation chemistry. This has evolved various highly efficient antibody attachment strategies to connect linker-payloads and targeting units with high chemical robustness, excellent in vivo stability and without affecting the targeting properties.

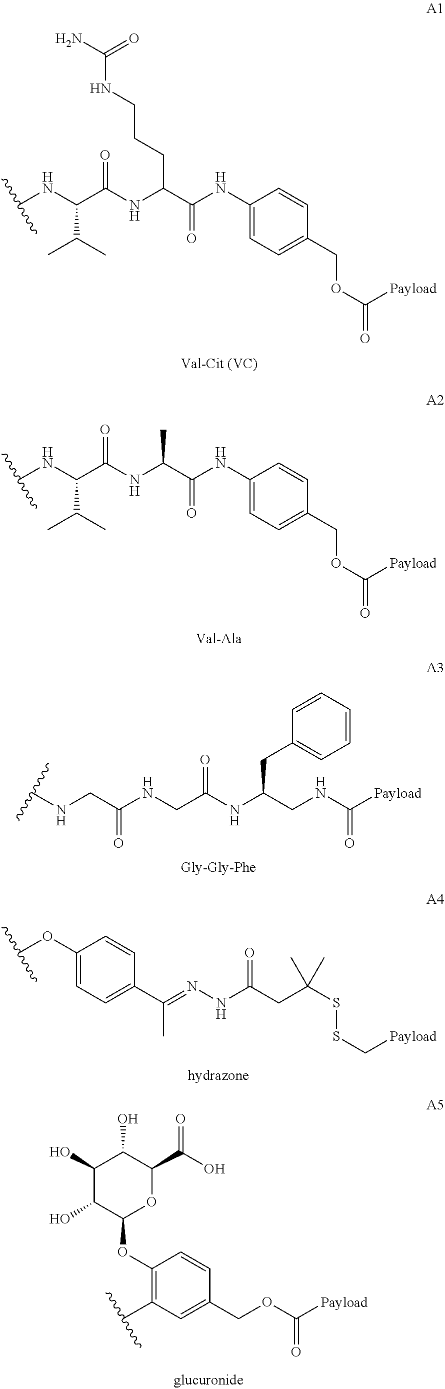

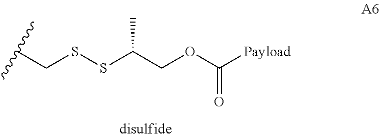

Chemical robustness and stability in the extracellular environment, combined with efficient, reliable, and high-turnover intracellular release of the therapeutic payload, are the central performance criteria that direct therapeutic success of antibody-drug conjugates. The choice and assembly of these units (such as the cleavable unit) within the flexible linker unit, and its self-immolative behaviour, are crucial for performance. In contrast to the extensive developments around the targeting unit and conjugation (also referred to as “bioconjugation”) chemistry, the choice of cleavable units has almost exclusively clustered around protease-labile oligopeptides. For example, cathepsin B is a ubiquitous cysteine protease that specifically cleaves the valine-citrulline dipeptide Val-Cit (VC). Thereby, cleavage of VC, when assembled in the VC-PABC A1 unit (cleavable unit linked to a self-immolative spacer), initiates the fragmentation-release cascade. VC-PABC A1 has been incorporated in the chemical design of various approved anti-cancer immunoconjugates, including Adcetris®, Lumoxiti®, Polivy®, Padcev®, Tivdak® (Fu et al. Signal Transduction and Targeted Therapy 2022, 7, 93). Other oligopeptidic linker units Val-Ala A2 or Gly-Gly-Phe A3 are found in the approved immunoconjugates Zylonta® and Enhertu®. The use of regular VC cleavable linkers may cause problems when plasma stability and protease specifity is intended for long circulation periods (days to weeks). Using adapted peptide sequences like Glu-Val-Cit improved its off-target cleavage resistance, but also further increased molecular complexity and molecular weight. Among the limited alternative units that are labile to chemical, hydrolytic or enzymatic cleavage and may initiate a fragmentation of the therapeutic payload from the antibody are hydrazones like A4 (found in the approved immunoconjugates Besponsa® and Mylotang®), glucuronides like A5, galactosides, arylsulfates, acetals, phosphates, nitrophenyls, or linear disulfides like A6. Most of these groups suffer from poor systemic plasma stability which limits their use and appearance in approved immunoconjugates (Mckertish et al., Biomedicines 2021, 9, 872).

Disulfide linker units, which so far have been limited to linear disulfides, have been used as cleavable linker units in immunoconjugates with generally high lability to reduction, that are known to be cleaved by monothiols such as glutathione (GSH) (Zhang et al. Drug Metabolism and Disposition 2019, 47, 1156). GSH is a highly abundant small redox-active peptide that is present intracellularly at high levels (ca. 10 mM). More importantly, in view of an application as cleavable linkers in immunoconjugates, GSH and other monothiol reducing peptides/proteins are also found at significant levels in the extracellular medium and blood plasma. This extracellular non-robustness poses a significant drawback for the use of linear disulfides for in vivo application in immunoconjugates. α-alkylation of linear disulfides decreased reducibility and moderately increased the plasma stability, but mechanistically these motifs lack the option for kinetic reversibility upon an initial thiol attack and are thus intrinsically instable (Su et al. Acta Pharmaceutica Sinica B 2021, 11, 12, 3889-3907). Until now, these factors limit the applicability of disulfides as cleavable units for immunoconjugates as their extracellular stability remains poor.

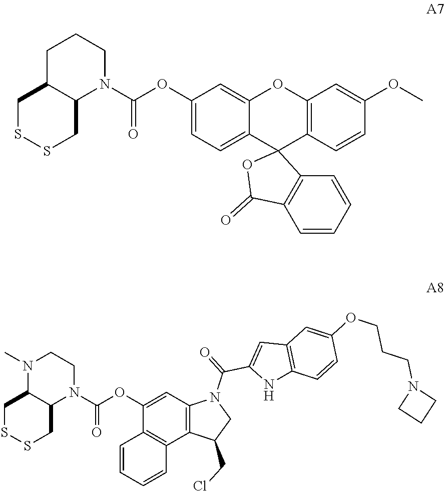

The use of dichalcogenides for small molecule drug activation has been almost entirely limited to linear disulfides that are intrinsically irreversibly cleaved by a single thiol nucleophilic addition. Recent advances in the development of novel cyclic disulfide units are based on bicyclic piperidine-fused 1,2-dithianes that combine thermodynamic stability and kinetic reversibility. Therefore, they are exclusively reduced intracellularly, and only by the strongest dithiol-type proteins, such as thioredoxin/glutaredoxin (Felber et al., J. Am. Chem. Soc. 2021, 143, 23, 8791; PCT/EP2022/057483), which possess specific activity in inflamed and/or tumorous environments. These piperidine dithiane reducible units were used to release and activate diagnostic agents for imaging redox enzyme activity in cell-free settings using a turn-on fluorogenic probe A7 (Felber et al., J. Am. Chem. Soc. 2021, 143, 23, 8791). Piperidine dithiane reducible units were also used in cells to control the release of a therapeutic cytotoxic agent from a bioreductive small molecule prodrug (e.g. molecules P10 and P21 in PCT/EP2022/059280).

However, it should be noted that these piperidine reducible units are not only “slow” to release (hours; Zeisel et al. ChemRxiv 2023, https://doi.org/10.26434/chemrxiv-2023-tm21m), but are also monofunctional, in that only a cargo molecule can be attached to them: there is no possibility of modifying them to feature a separate targeting moiety.

Instead, bicyclic piperazine-fused 1,2-dithianes have been used in cells to liberate a therapeutic cytotoxic agent of the duocarmycin family from e.g. the bioreductive small molecule prodrug A8 (Felber et al. ACS Cent. Sci. 2023, 9, 4, 763; PCT/EP2022/059280). As reducible units within small molecule constructs, piperazine-fused cyclic disulfides exhibited faster payload-releasing properties as compared to their piperidine-fused counterparts.

Crucially however, the molecular designs used in those reports were restricted to the use of only small molecule side chain attachments (e.g., —Ac, -Me, —H) on the free piperazine nitrogen, presumably since large molecular attachments near the —SS-bond (such as, e.g., biomacromolecules) were assumed to counteract cellular entry and/or reductive turnover and therefore to block performance.

Recently, piperazine-fused 1,2-dithianes have also been reported to release fluorophore cargos, while keeping their reduction-selectivity profile for dithiol reductases of the thioredoxin family (Zeisel et al. ChemRxiv 2023, https://doi.org/10.26434/chemrxiv-2023-tm21m). In this report, it was showcased that the piperazine's second nitrogen can be substituted to feature a second moiety: such that the second moiety may add additional functionality, although the release of the active cargo should still be directed by the piperazine-disulfide. Reactive groups that can serve as synthetic handles for assembling extended fluorogenic constructs, such as NHS-esters (A9), α-haloamides (A10) or aldehydes (A11), were reported as the second moiety; also, small molecule moieties were included as the second moieties (-Me A12, 4-methylpiperazinyl-4-butanamide A13, and glutathione-derived tripeptide A14).

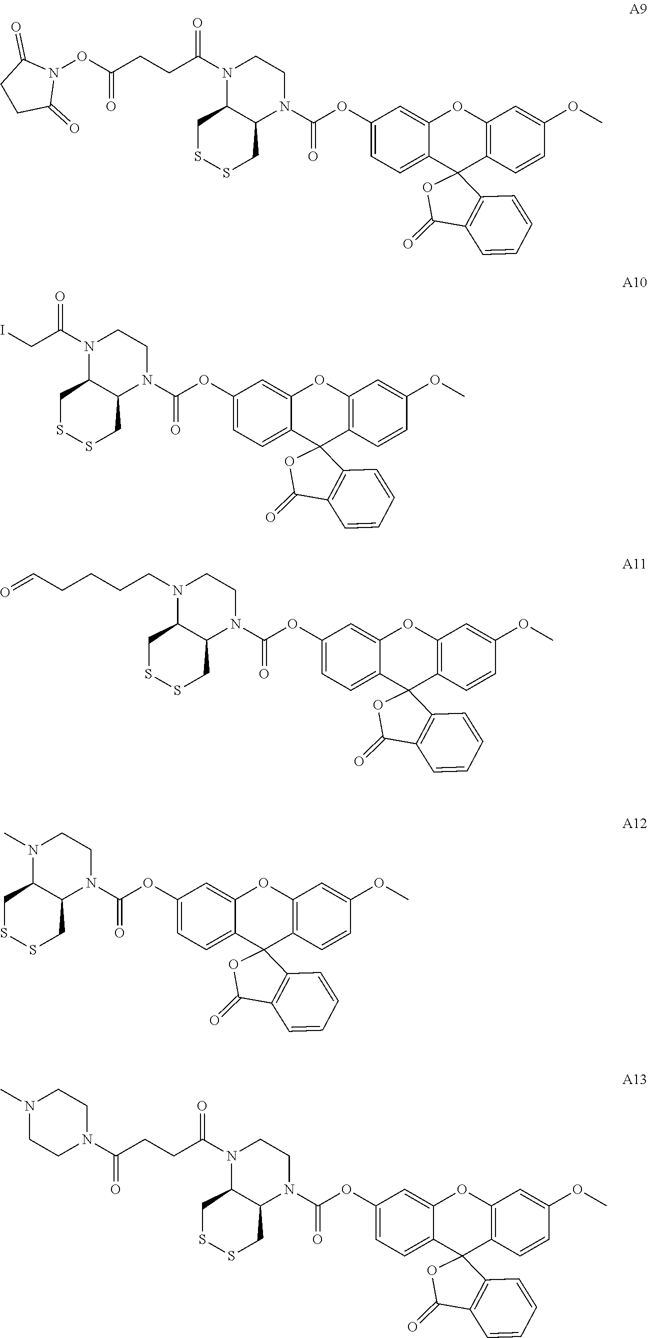

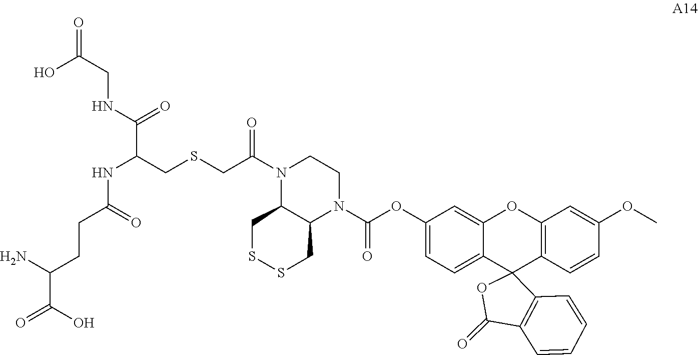 [SMILES: COc1ccc2c(c1)Oc1cc(OC(=O)N3CCN(C(=O)CSCC(NC(=O)CCC(N)C(=O)O)C(=O)NCC(=O)O)[C@@H]4CSSC[C@@H]43)ccc1C21OC(=O)c2ccccc21]

However, once again, only very small molecule groups were postulated as the possible second moieties in these fluorogenic designs. The performance of any cyclic dichalcogenide, including piperazine-fused cyclic disulfides, as a reducible and cleavable unit for releasing active agents (including fluorophores or therapeutics or other cargoes) from large biomolecular conjugates, was never yet tested: in all these reports, cyclic disulfides were limited to liberating cargoes from small molecular probes and prodrugs (typified as “monoprodrugs” in the scheme below). Furthermore, the trends in the relevant prior art since cyclic dichalcogenides first began to be used as reducible triggers for cargo release (from ca. 2014, as described in Felber et al., PCT/EP2022/057483), teach that only small molecule side chains could feasibly be tolerated without sacrificing the effectiveness of reduction by cellular reductases, since (a) despite ten years of publications, no biomolecular conjugates were reported; and (b) specific examples such as the glutathione conjugate A14 (which had been reported to show “a small kinetic Grx1-preference” relative to non-glutathionylated analogues such as A13) can be plausibly interpreted as showing that specificity for a desired spectrum of reductants is sacrificed by the presence of even a small second moiety (here, a tripeptide), which argues against the logic of the present invention (that discovers that the second moiety can in fact be designed and chosen flexibly, to install targeting without losing an otherwise favourable reductant profile).

It was one object of this invention to provide a novel class of fused cyclic dichalcogenide-based cleavable units for high-performance payload release in antibody-drug conjugates (ADCs), immunoconjugates, and other bifunctional targeted therapeutic constructs. We disclose that ring-fused cyclic disulfides are particularly suitable as such cleavable units, with unexpectedly fast reduction-to-release kinetics that may even benefit from the sterically-pressured environment of the cleavable unit when it is contained in a macromolecular conjugate (typified as “immunoconjugate” in the scheme below). These dichalcogenides will also be similarly or even more robust against reductive and non-reductive extracellular cleavage than their small molecule analogues (“monoprodrug” in the scheme below). Fused cyclic dichalcogenide cleavable units will allow for high-performance use in large constructs, e.g., antibody-drug conjugates, and are compatible with a variety of state-of-the-art antibodies (or other targeting units), bioconjugation techniques, self-immolative spacers, and therapeutic payloads. Notably, the molecular feature that primarily dictates the release behaviour of the molecules of the invention is the dichalcogenide cleavable unit they contain.

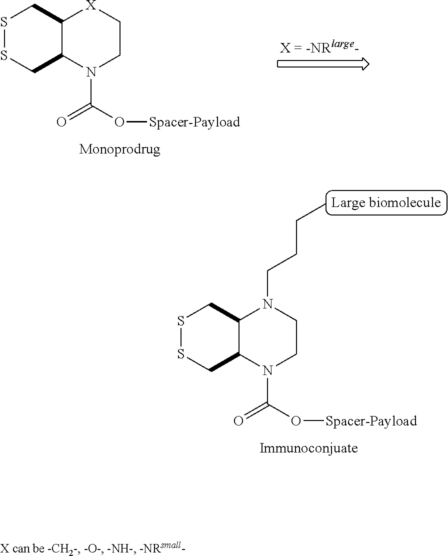

In macromolecular immunoconjugates, the fused cyclic dichalcogenide cleavable units of the invention will confer superior properties as compared to state-of-the-art cleavable units, including one or more the following specific characteristics:
1) Fast, high-turnover intracellular reductive cleavage, liberation and activation of the therapeutic payload from the immunoconjugate upon intracellular reduction primarily mediated by dithiol-type reductases of the thioredoxin family;
2) Chemical robustness to extracellular hydrolysis, extracellular proteolysis, or extracellular reduction, as mediated through typical levels of extracellular species (e.g. proteases, esterases, monothiols, reductases), which is ensured by the highly stable carbamate linkages at the cleavable unit and by the reversibility of monothiol addition to fused cyclic dichalcogenides: so evading decomposition in the blood stream and the extracellular space;
3) Full modularity of the bicyclic dichalcogenide cleavable units for the use in large multicomponent conjugates with the flexibility to independently combine with self-immolative spacers and chemical bioconjugation warheads of choice. These combinations are chemically robust so that for compounds of the invention only the reduction-mediated cleavage of the piperazine dithiane moiety directs the payload release and, ultimately, the therapeutic benefit of these constructs.

It was a further object of the present invention to provide macromolecular therapeutic constructs which are capable of releasing a molecular payload from an immunoconjugate after reductive cleavage, and which
(A) are suitable for treating, ameliorating or preventing a disorder associated with disulfide reductive activity; and/or
(B) are suitable for treating, ameliorating or preventing a disorder selected from a neoplastic disorder, particularly cancer; atherosclerosis; an autoimmune disorder; an inflammatory disease; and a chronic inflammatory autoimmune disease; and/or
(C) are suitable for treating, ameliorating or preventing a disorder associated with a dysregulated redox state and/or with hypoxia and/or with activation of the hypoxic response pathway; and/or
(D) are useful because they are efficiently activated upon delivery into cells and thus are useful as cleavable units.

### SUMMARY OF THE INVENTION

The present invention refers to a compound having the formula (I)

T-K-A-L-B (I)

The compound having the formula (I) is a multicomponent immunoconjugate that is capable of targeting a cellular environment and releasing a molecular cargo B in the presence of a reductant, and is thus suitable for treating, ameliorating or preventing a disorder selected from a neoplastic disorder, particularly cancer; atherosclerosis; an autoimmune disorder; an inflammatory disease; and a chronic inflammatory autoimmune disease.

Precursors of the claimed compound having the formula (I) are compounds having the formulae (II), (III), (IV) and (V):

K′-A-L-B (II)

K′-A-L′ (III)

Q¹-D²-D¹-A-L-B (IV)

K″-A-L′ (V)

Stereoisomers, racemic mixtures, pharmaceutically acceptable salts, esters, hydrates, or solvates of the compounds having the formulae (I), (II), (III), (IV) and (V) are also within the scope of the present invention.

The key to the invention is the integration of the redox-sensitive structure-A-which is used to control both the integrity of the assembled conjugate and its release of the molecular cargo -B that is conditional upon the reductive stimulus of interest, with the aim to ensure selective delivery (e.g. protection of healthy tissues, as outlined above). The choices of molecular cargo -B and of linking group -L- and of targeting unit T- and of linker -W- are not particularly limiting: for example, the cargo B can be chosen flexibly according to the effect that a compound of the invention shall deliver, and the targeting unit can be chosen according to the type of biomolecule and attachment that are appropriate.

### DESCRIPTION OF THE FIGURES

FIG. 1 indicates the results of anti-proliferation cellular assays using the compounds of the invention (examples 4 and 5). The anti-proliferative activity of an immunoconjugate reflects its antigen-mediated uptake into the antigen-positive cell line and the intracellular release of the therapeutic payload. This experiment compares the compounds of the invention that are based on either the trans-fused or the cis-fused piperazine 1,2-dithiane-based reductively cleavable unit in a HER2-positive or HER2-negative cell line. The receptor mediated uptake primarily dictates the activity of the examples 4 and 5 with a strong shift between antigen-positive and antigen-negative cell lines. Considering the high potency of the active payload of the duocarmycin family in these constructs these results indicate good stability of the reductively cleavable unit in cell media. Moreover, the overall stronger activity of trans-fused piperazine derived immunoconjugates compared to their cis-fused counterparts in targeted settings indicates faster intracellular release of the active payload. The data for HER2-negative cell lines supports higher robustness of the cis-fused ADC towards off-target drug release.

FIG. 2 indicates the results of anti-proliferation cellular assays using the compounds of the invention (examples 6 and 7). The anti-proliferative activity of an immunoconjugate reflects its antigen-mediated uptake into the antigen-positive cell line and the intracellular release of the bioactive payload. This experiment compares the compounds of the invention 6 and 7 that are based on the same cleavable unit and the same therapeutic payload, but on two different antibodies targeting different antigens. The activity of examples 6 and 7 is determined in two different cell lines that are either Trop-2- or CD20-positive. A strong shift in activity between targeted and untargeted immunoconjugate reflects the matching combination of antibody with its antigen-positive cell line, for example 6 targeting Trop-2, and 7 targeting CD20. The low activity of 6 in a CD20-positive cell line and 7 in Trop-2-positive cell line cross-validates the stability and robustness of the cleavable linker unit.

FIG. 3 indicates the results of an in vivo pharmacokinetics experiment in mice comparing the compounds of the invention (examples 6 and 7) with the approved immunoconjugate Trodelvy® that is based on the same therapeutic payload but uses a different cleavable unit strategy for intracellular release. The results show a fast and significant loss of intact immunoconjugate in mouse plasma already after a short period of time for Trodelvy®. However, for examples 6 and 7, the immunoconjugates are intact for much longer in mouse plasma, indicating the high chemical robustness of the reductively cleavable unit in an extracellular environment, and permitting longer-lasting antibody-mediated delivery of the therapeutic cargo to the target tissue, as well as lower side-effects from cargo release in plasma.

FIG. 4 indicates the results of an anti-cancer efficacy study in mice with a Trop-2-positive breast cancer cancer cell line comparing the compound of the invention 6 with the approved immunoconjugate Trodelvy®. The results show a strong suppression of tumor growth in the in vivo model for all immunoconjugates (single treatment at day 7) as compared to the vehicle group that did not receive treatment at day 7. For mice receiving Trodelvy® tumors significantly start to grow again after day 30. This effect presumably reflects the high lability of the cleavable unit used in Trodelvy® in mouse serum as indicated by results shown in FIG. 3. However, longer-lasting tumor regression is determined for 6, reflecting the high chemical robustness of the cleavable unit in vivo.

### DEFINITIONS

Unless stated otherwise, the following definitions apply.

The term “cleavable unit” refers refers to any molecular motif that covalently connects two parts of a larger (bio) molecular construct and that contains a potentially reactive site where, after a chemical or enzymatic reaction at this site, a reaction is initiated which irreversibly disconnects the two parts of the construct. Examples thereof are to be found below.

The term “functional group” is used in the sense that is understood by those skilled in the art, to indicate a small chemical motif or a protected variant of the same, that can participate in a reaction with another chemical motif (usually of a different nature). Examples of a “functional group” in this sense include an amine, a carboxylic acid, an alkyne, etc.

The term “immunoconjugate” refers to a conjugate of a targeting unit with a drug through any bioconjugation method known in the art, such as a chemotherapeutic agent, a toxin, an immunotherapeutic agent. A linker unit is any chemical moiety that links an antibody or an antigen binding fragment covalently to the drug.

The term “antibody-drug conjugate” (abbreviated as “ADC”) refers to a conjugate of an antibody or an antigen binding fragment with a drug through any bioconjugation method known in the art, such as a chemotherapeutic agent, a toxin, an immunotherapeutic agent. A linker unit is any chemical moiety that links an antibody or an antigen binding fragment covalently to the drug.

The term “targeting unit” refers to an antibody, functional fragment, antigen-binding antibody fragment, antigen-binding region, antibody fragment, Fynomer®, antibody mimetic, affibody, adnectin, anticalin, DARPin, avimer, nanofitin, affilin, Kunitz domain peptide, trispecific binding molecule, or probody.

The term “antibody” refers to immunoglobulin molecules, preferably comprised of four polypeptide chains, two heavy (H) chains and two light (L) chains which are typically inter-connected by disulfide bonds. Depending on the amino acid sequence of the constant domain of their heavy chains, intact antibodies can be assigned to different “classes”. There are five major classes of intact antibodies: IgA, IgD, IgE, IgG, and IgM, and several of these may be further divided into subclasses. A preferred class of immonoglobulins for use in the present invention is IgG. A “functional fragment” or “antigen-binding antibody fragment” of an antibody/immunoglobulin hereby is defined as a fragment of an antibody/immnuoglobulin that retains the antigen-binding region. An “antigen-binding region” of an antibody typically is found in one of mor hyper variable region(s) of an antibody. “Functional fragments”, “antigen-binding antibody fragments”, or “antibody fragments” of the invention include but are not limited to Fab, Fab′, Fab′-SH, F(ab′)₂, and Fv fragments; diabodies; fusion proteins; single domain antibodies (Dabs), linear antibodies, single-chain antibody molecules; and multispecific, such a bi- and tri-specific, antibodies formed from antibody fragments, a fragment(s) produced by a Fab expression library, or an epitope-binding fragment(s) of any of the above which immunospecifically bind to a target antigen (e.g., a cancer cell antigen, a viral antigen or a microbial antigen).

The term “antibody fragment” refers to a portion of an intact antibody comprising the antigen-binding or variable region thereof. To be of use in the present invention, the antibody fragment should have the requisite number of sites for attachment to a drug-linker.

The terms “monoclonal antibody” and/or “human/humanized antibody” refer to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

The term “nanobody” refers to an antibody-derived therapeutic protein that contains a heavy-chain domain of an antibody with its unique structural and functional properties but with sigificantly reduced moelcular size

As used herein, the term “antibody mimetic” refers to compounds which, like antibodies, can specifically bind antigens, but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunitz domain peptides, Fynomers®, trispecific binding molecules and prododies. These polypeptides are well known in the art and are described in further detail herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10th extracellular domain of human fibronectin III (10Fn3), which adopts an g-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity, i.e. against HLA-J, can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci USA. 96 (5): 1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity, i.e. for HLA-J, can be selected, for example, by phage display techniques. The binding specificity of the different A-domains contained in an avimer may but does not have to be identical (Weidle U H, et al., (2013), Cancer Genomics Proteomics; 10 (4): 155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule, such as e.g. HLA-J, by randomising the amino acids on the binding surface (Mouratou B, Béhar G, Paillard-Laurance L, Colinet S, Pecorari F., (2012) Methods Mol. Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity, i.e. against HLA-J, is effected, for example, by phage display or ribosome display techniques. Depending on the scaffold, affilins have a molecular weight of approximately 10 or 20 kDa. As used herein, the term affilin also refers to di- or multimerised forms of affilins (Weidle U H, et al., (2013), Cancer Genomics Proteomics; 10 (4): 155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDa and domains with the required target specificity, i.e. against HLA-J, can be selected by display techniques such as phage display (Weidle et al., (2013), Cancer Genomics Proteomics; 10 (4): 155-68).

As used herein, the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20 (2): 57-68, Gebauer and Skerra (2009) Curr Opinion in Chemical Biology 13:245-255, or Schlatter et al. (2012), MAbs 4:4, 1-12).

The term “bispecific binding molecule” or “chimeric antibody” as used herein refers to a polypeptide molecule that possesses two binding domains and is thus capable of binding, preferably specifically binding to two different epitopes. The term “trispecific binding molecule” as used herein refers to a polypeptide molecule that possesses three binding domains and is thus capable of binding, preferably specifically binding to three different epitopes. At least one of these two/three epitopes is a target epitope of molecules of the invention. The two other epitopes may also be target epitopes or may be epitopes of one or two different antigens.

As used herein, the term “probody” refers to a protease-activatable antibody prodrug. A probody consists of an authentic IgG heavy chain and a modified light chain. A masking peptide is fused to the light chain through a peptide linker that is cleavable by tumor-specific proteases. The masking peptide prevents the probody binding to healthy tissues, thereby minimizing toxic side effects.

The term “bioconjugation warhead” refers to reactive chemical functional groups that allow for conjugation of the targeting unit to the linker-drug unit, resulting in a functional immunoconjugate. These functional groups are well-known and bioconjugation may be achieved by any method known in the art.

The term “alkyl” refers to a saturated straight or branched hydrocarbon chain. In any of the below definitions, C₁₋₄-alkyl is not particularly limited, and refers to a saturated straight or branched hydrocarbon chain, which has 1 to 4 carbon atoms, preferably 1 to 3 carbon atoms, more preferably 1 or 2 carbon atoms, even more preferably 1 carbon atom. Preferably C₁₋₄-alkyl can be methyl, ethyl, n-propyl, or i-propyl, more preferably it can be methyl or ethyl, most preferably it can be methyl.

In any of the below definitions, C₂₋₄-alkylene is not particularly limited, and refers to a saturated straight or branched hydrocarbon chain, which has 2 to 4 carbon atoms, preferably 2 or 3 carbon atoms, more preferably 2 carbon atoms. Preferably C₂₋₄-alkylene can be ethylene, or n-propylene, more preferably it can be ethylene.

An aromatic ring is any not particularly limited and refers to any aromatic carbocyclic ring, which has 5 to 7 carbon atoms, preferably 5 or 6 carbon atoms, more preferably the aromatic ring is phenyl. The aromatic ring can optionally be annulated to one or more carbocyclic or heterocyclic rings. Examples of annulated rings include but are not limited to naphthyl, quinolyl, isoquinolyl, quinoxalyl, benzopyranonyl, benzo[1,3]dioxolyl, benzothiazolyl, 2,3-dihydro-1H-benzo[e]indole, xanthenyl or indolyl, preferably naphthyl, quinolyl or xanthenyl.

The heteroaromatic ring is any not particularly limited and refers to any aromatic heterocyclic ring, which has 5 to 7 ring atoms, preferably 5 or 6 ring atoms. The heteroaromatic ring can optionally be annulated to one more carbocyclic or heterocyclic rings. Examples of the heteroaromatic ring include thiophenyl, pyridinyl, pyrazolyl, pyrimidinyl, purinyl, pyrrolo, furanyl, oxazolyl, thiazolyl, imidazolyl, benzofuranyl, benzothiophenyl, benzothiazolyl, benzoxazolyl or naphthyridinyl, preferably pyrrolo, pyridinyl, thiophenyl or furanyl.

An aliphatic moiety is a saturated or unsaturated, linear, branched or cyclic moiety containing between 1 to 80 carbon atoms and optionally 0 to 19 heteroatoms, preferably 0 to 15 heteroatoms, wherein the heteroatoms are typically chosen from O, N, S, Se, Si, Hal, B or P, preferably chosen from O, N, Hal, S, Si or P, more preferably O, N, Hal, S, Si, even more preferably chosen from O, N or Hal.

A carbocyclic ring is a cyclic structure containing from 4 to 15 carbon atoms. Examples of the carbocyclic ring include cyclobutane, cyclopentane, cyclohexane, benzene, cycloheptane, naphthalene or anthracene, preferably cyclohexane, cyclopentane, benzene or naphthalene, more preferably benzene or naphthalene.

A heterocyclic ring is a cyclic structure containing at least one heteroatom selected from N, O, Si, S, B, P, and Se, and containing at least one carbon atom. Examples of the heterocyclic ring include thiophene, pyridine, pyrazole, pyrimidine, pyrrole, furan, oxazole, thiazole, imidazole, quinoline, isoquinoline, benzofuran, benzothiophene, benzothiazole, benzoxazole, naphthyridine, piperidine, piperazine, pyrrolidine, tetrahydrothiophene, tetrahydrofuran or pyran, preferably pyridine, furan, thioazole, quinoline, isoquinoline, benzothiazole, piperidine, piperazine or pyran, more preferably pyridine, quinoline, piperidine or piperazine.

Halogen refers to —F, —Cl, —Br or —I, preferably —F or —Cl.

The term “leaving group” refers to a chemical moiety that detaches from a larger chemical structure while breaking the covalent bond and leaving a chemical fragment behind. The electron pair of the bond remains with the leaving group. In this process the leaving group may be replaced by a new chemical moiety that is then covalently linked to the chemical fragment. As used herein, a leaving group may be used to activate a chemical structure for the intended conjugation with a certain other chemical structure, e.g., for bioconjugation of a chemical warhead and amino acid side chains of antibodies, or else, for connecting different units of a linker between an antibody and its therapeutic payload in an immunoconjugate.

If a moiety is referred to as being “substituted” by a substituent it can in each instance include one or more of the indicated substituents. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclisation, elimination etc., As used herein, the term “substituted” is contemplated to include all permissible substitutents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such es a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthiol, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

Disulfide reductase (also referred to as disulfide oxidoreductase) refers to an enzyme capable of reducing dichalcogenides to the corresponding dichalcogenols. Examples thereof include thioredoxin reductase 1 (TrxR1), TrxR2, thioredoxin glutathione reductase (TrxR3/TGR), endoplasmic reticulum resident protein 18 (ERp18), ERp44, ERp57, ERp72, ERdj5, methionine-R-sulfoxide reductase (MsrB1), protein disulfide isomerase A 1 (PDIA1), PDIA2, PDIA3, PDIA4, PDIA5, PDIA6, EndoPDI, PDIp, disulfide bond formation protein A (DsbA), DsbB, DsbC, or glutathione disulfide reductase (GR). A disulfide effector protein (or redox effector protein or simply effector protein) is a redox active protein, that bears a reducing thioredoxin-like domain and that is itself reductively enabled by an upstream disulfide oxidoreductase enzyme. Examples thereof include thioredoxin 1 (Trx1), Trx2, thioredoxin-related protein of 14 kDa (TRP14), thioredoxin-related transmembrane protein 1 (TMX1), TMX2, TMX3, TMX4, thioredoxin-like protein 1 (TXNL1), human macrothioredoxin (hMTHr), anterior gradient protein 2 (AGR2), AGR3, glutaredoxin 1 (Grx1), Grx2, Grx3, Grx4, Grx5, GrxA or Grx6.

The term “cancer”, as used herein, refers to a group of proliferative diseases characterized by uncontrolled division of abnormal cells in a subject. Non-limiting examples of cancers include acoustic neuroma, adenocarcinoma, angiosarcoma, basal cell carcinoma, bile duct carcinoma, breast cancer, bronchogenic carcinoma, cervical cancer, bladder carcinoma, chondrosarcoma, chordoma, choriocarcinoma, craniopharyngioma, cystadenocarcinoma, embryonal carcinoma, endotheliosarcoma, ependymoma, epithelial carcinoma, Ewing's tumor, fibrosarcoma, hemangioblastoma, leiomyosarcoma, liposarcoma, Merkel cell carcinoma, melanoma, mesothelioma, myelodysplastic syndrome, myxosarcoma, oligodendroglioma, osteogenic sarcoma, ovarian cancer, pancreatic cancer, papillary adenocarcinomas, papillary carcinoma, pinealoma, prostate cancer, renal cell carcinoma, retinoblastoma, rhabdomyosarcoma, sebaceous gland carcinoma, seminoma, squamous cell carcinoma, sweat gland carcinoma, synovioma, testicular tumor, Wilms' tumor, adrenocortical carcinoma, urothelial carcinoma, gallbladder cancer, parathyroid cancer, Kaposi sarcoma, colon carcinoma, gastrointestinal stromal tumor, anal cancer, rectal cancer, small intestine cancer, brain tumor, glioma, glioblastoma, astrocytoma, neuroblastoma, medullary carcinoma, medulloblastoma, meningioma, leukemias including acute myeloid leukemia, multiple myeloma, acute lymphoblastic leukemia, liver cancer including hepatoma and hepatocellular carcinoma, lung carcinoma, non-small-cell lung cancer, small cell lung carcinoma, lymphangioendotheliosarcoma, lymphangiosarcoma, primary CNS lymphoma, non-Hodgkin lymphoma, and classical Hodgkin's lymphoma.

The term “therapeutic cargo” or “therapeutic payload”, as used herein, represents a therapeutic agent to be delivered to a site of enzymatic activity or disease (e.g. a tumour, a site of inflammatory or autoimmune disease). Cargos used in the compounds of the invention may be small molecules. Examples include agents for treating, ameliorating, or preventing a neoplastic disorder, an inflammatory disorder (e.g. a non-steroidal anti-inflammatory drug (NSAID), a disease-modifying anti-rheumatic drug (DMARD), or mesalamine), atherosclerosis; an autoimmune disorder; a chronic inflammatory autoimmune disorder; ischaemia; and reperfusion injury as well as kinase inhibitors. Examples also include therapeutically acceptable DNA-alkylating agents, DNA-intercalating agents or tubulin-inhibiting agents. Examples particularly include “cytotoxic therapeutic agents” as listed below.

The term “pharmaceutically acceptable salt” refers to a salt of a compound of the present invention. Suitable pharmaceutically acceptable salts include acid addition salts which may, for example, be formed by mixing a solution of compounds of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, hemisulfate, heptanoate, hexanoate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, succinate, tannate, tartrate, teoclate, tosylate, triethiodide, undecanoate, valerate, and the like (see, for example, S. M. Berge et al.; J. Pharm. Sci. 1977, 66, 1-19).

When the compounds of the present invention are provided in crystalline form, the structure can contain solvent molecules. The solvents are typically pharmaceutically acceptable solvents and include, among others, water (hydrates) or organic solvents. Examples of possible solvates include hydrates, ethanolates and iso-propanolates.

The term “pharmaceutically acceptable ester” refers to an ester of a compound of the present invention. Suitable pharmaceutically acceptable esters include acetyl, butyryl, and acetoxymethyl esters.

The term “proteinogenic amino acid” herein comprises the 20 principal naturally-occurring L-amino acids (glycine, alanine, valine, leucine, isoleucine, phenylalanine, proline, methionine, serine, threonine, cysteine, tyrosine, tryptophan, asparagic acid, glutamic acid, asparagine, arginine, histidine, lysine, asparagine and glutamine) which are to be attached, as the monopeptide, via their carboxyl terminus to the specified amine nitrogen of the compound, creating amides. Preferably the proteinogenic amino acid is selected from leucine, serine and lysine.

The term “self-immolative spacer”, also called “traceless linker” or “self-immolative linker”, as used herein, represents a multivalent (e.g., divalent) group covalently linking a dichalcogenide-containing group which will be further defined below (as the moiety A), to a cargo B, in such a way that reductive cleavage of the dichalcogenide bond in the moiety A is followed by a sequence of reactions which result in the cleavage of a bond between the self-immolative linker group and the cargo B, thereby releasing the cargo.

The stereochemistry at the ring atoms bridging the two rings of the bicyclic dichalcogenide structure of the compounds of formula (I)-(V) of the invention is not particularly limited; any absolute configuration of the two bridging ring tertiary carbon atoms is within the scope of the compounds of formula (I)—(V). Either carbon can be separately either R, or S, or a mixture of R and S in any proportions (including racemic and undefined proportions); therefore the dichalcogenide ring may be considered to be cis-fused to the carbamate-containing ring with any given stereochemistry, or trans-fused to the carbamate-containing ring with any given stereochemistry, or present as a mixture in any proportions of cis-fused and trans-fused with any given stereochemistries (including racemic and undefined proportions). Preferably, the disulfide ring is either cis-fused or trans-fused.

The term “cytotoxic therapeutic agent” refers to compounds that have antineoplastic and/or antitumoral efficacy and which are typically useful in the treatment of cancer, optionally as part of combination therapies. Such agents include topoisomerase inhibitors, duocarmycins, pyrrolobenzodiazepines, trioxacarcins, nitrogen mustards, calicheamycins, mitomycins, doxorubicin derivatives, toxoids, auristatins, tubulysins, folic acid analogues, nitrosoureas, pyrimidines, tamoxifens, androgens, maytansinoids, aziridines, methylamelamines, platinum complexes, bifunctional PROTAC degraders, and molecular glue degraders. Illustrative examples of the therapeutic agent A¹-OH, A²-NH-A³, A⁴-SH, or A⁵-C(O) OH include camptothecin, topotecan, NH₂-bicyclo[1.1.1]pentane-7-MAD-MDCPT, deruxtecan (Dxd), exatecan, Gly-cyclopropane-exatecan (HY-13631), 10-hydroxycamptothecin, 10-hydroxybelotecan, belotecan, 10-hydroxygimatecan, gimatecan, CKD-602, BNP-1350, sinotecan, 7-ethyl-10-hydroxy-camptothecin (SN-38), 10-hydroxy-20-acetoxy-camptothecin, irinotecan (CPT-11), RFS 2000, duocarmycin A, duocarmycin SA, seco-duocarmycin, duocarmycin DM, duocarmycin GA, duocarmycin MB, duocarmycin MA, seco-DUBA, duocarmycin DC1, duocarmycin DC4, CBI-TMI, DC0-NH2, CC-1065, adozelesin, KW-2189, carzelesin, bizelesin, 5-hydroxy-seco-cyclopropabenzaindoles, 5-hydroxy-seco-(2-methyl-cyclopropa)benzaindoles, 5-hydroxy-seco-cyclopropamethoxybenzaindoles, 5-amino-seco-cyclopropabenzaindoles, C16-modified trioxacarcins, DC-45-A1, DC-45-A2, trioxacarcin A, trioxacarcin C, trioxacarcin D, LL-D49194α1, pyrrolobenzodiazepine, chlorambucil, 4-(bis(2-chloroethyl)amino)phenol, 4-(bis(2-bromoethyl)amino)phenol, 4-((2-chloroethyl-2′-mesylethyl)amino)phenol, 4-(bis(2-mesylethyl)amino)phenol, chlornaphazine, cyclophosphamide, estramustine, ifosfamide, mechlorethamine, mechlorethamine oxide, melphalan, novembichin, phenesterin, prednimustine, trofosfamide, uracil mustard, calicheamycin, calicheamycin α1, calicheamycin β1, calicheamycin γ1, calicheamycin ω1, calicheamycin T, calicheamycin δ1, calicheamycin ε1, dynemicin, dynemicin A, carabicin, carzinophillin, actinomycin, antrmycin, bleomycin, cactinomycin, carninomycin, chromomycin, dactinomycin, marcellomycin, mitomycin C, mycophenolic acid, nogalamycin, olivomycin, peplomycin, potfiromycin, puromycin, quelamycin, clodronate, esperamicin, neocarzinostatin, aclacinomysin, azaserine, daunorubicin, detorubicin, 6-diazo-5-oxo-L-norleucine, doxorubicin (Adrlimycin®), morpholino-doxorubicin, cyanomorpholino-doxorubicin, 2-pyrrolino-doxorubicin, deoxydoxorubicin, DMEA-PNU-159682, doxorubicin-N,O-acetal, pirarubicin, epirubicin, esorubicin, rodorubicin, tubercidin, zorubicin, daunomycin, streptomigrin, streptozotocin, ubenimex, valrubicin, denopterin, methotrexate, pemetrexed, raltitrexed, pteropterin, trimetrexate, fludarabine, 6-mercaptopurine, thiamiprine, thiguanine, folinic acid, aceglatone, aldophophamide glycoside, aminolevulinic acid, eniluracil, amsacrine, bestrabucil, bisantrene, edatrexate, defofamine, demecolcine, diaziquone, elfornithine, elliptinium acetate, etoglucid, hydroxyurea, lentinan, lonidainine, carmustine, chlorozotocin, fotemustine, lomustine (CCNU), nimustine, ranimnustine, ancitabine, azacytidine, 6-azauridine, carmofur, cytarabine, dideoxyuridine, doxifluridine, enocitabine, gemcitabine, 6-thioguanine, troxacitabine, 5-azacytidine, 5-fluordeoxyuridine, cytarabine, cladribine, clofarabine, decitabine, floxuridine, nelarabine, tegafur, tioguanine, calusterone, dromostanolone, propionate, epitiostanol, mepitiostane, testolactone, aminoglutethimide, mitotane, trilostane, maytansinoids, maytansine DM1, maytansine DM3, N-Me-L-Ala-maytansinol, ansamitocin, mitoguazone, mitoxantrone, mopidanmol, nitraerine, pentostatin, phenamet, losoxantrone, 2-ethylhydrazide, procarbazine, razoxane, rhizoxin, sizofiran, spirogermanium, tenuazonic acid, triaziquone, trichothecene, T-2 toxin, verracurin A, roridin A, anguidine, urethane, dacarbazine, mannomustine, mitobronitol, mitolactol, pipobroman, gacytosine, arabinoside, cyclophosphamide (Cytoxan®), busulfan, improsulfan, piposulfan, benzodopa, meturedopa, uredopa, carboquone, altretamine, triethylenemelamine, triethylenephosphoramide, triethylenethiophosphoramide, trimethylolomelamine, bullatacin, bullatacinone, ribociclib, erlotinib (Tarceva®), bortezomib (Velcade®), fulvestrant (Faslodex®), letrozole (Femara®), imatinib (Gleevec®), sutent (SU11248), PTK787/ZK 222584, oxaliplatin (Eloxatin®), cisplatin, carboplatin, 5-fluorouracil (5-FU), capecitabine, leucovorin, rapamycin (Rapamune®), lapatinib (Tykerb®), lonafamib, sorafenib (Bay43-9006), gefitinib (Iressa®), baricitinib, filgotinib, tofacitinib, upadacitinib, ruxolitinib, peficitinib, decemotinib, solcitinib, itacitinib, fostamatinib, Ivarmacitinib (SHR0302), AG1478, AG1571, thiotepa, MT-802, SJF620, P131, L181, CJH-005-067, DD-04-015, DD-03-171, MZ-1, SNIPER (BRD4)-1, 1-208 (Kymera), dBET1, BETd-260/ZBC260, A1874, QCA570, ARV-771, ARV-825, ARV-110, ARV-471, DT2216, MS40, SJF-α, SJF-δ, 753b, SIM1, I-8, DP-C-1, CFT7455, eragidomide (CC-90009), NVP-DKY709, E7820, (R)-CR8, TMX-4116, NRX-252114, BI-3802, mezigdomide (CC-92480), thalidomide, lenalidomide, pomalidomide, avodomide (CC-122), 5-hydroxythalidomide, FPFT-2216, CC-647, CC-3060, iberdomide, CC-885, ZXH-1-161, indisulfam, paclitaxel (Taxol®), docetaxel (Taxotere®), cabazitaxel, epothilone A, epothilone B, epothilone, etoposide, teniposide, eleutherobin, vinblastine, vincristine, vindesine, vinorelbine, novantrone, edatrexate, aminopterin, xeloda, ibandronate, difluoromethylornithine (DFMO), retinoic acid, capecitabin, monomethyl auristatin E (MMAE), monomethyl auristatin F (MMAF), monomethyl auristatin D (MMAD), auristatin, PF-06380101 (auristatin-0101), duostatin, dolastatins, dolastatin 10, dolastatin 15, symplostatin 1, symplostatin 3, symplostatin 4, auristatin PE, zinostatin, spongistatin, tubulysin IM-3, tubulysin A, tubulysin B, tubulysin C, tubulysin G, tubulysin I, tamoxifen (Nolvadex®), raloxifene, droloxifene, 4-hydroxytamoxifen, trioxifene, keoxifene, LY117018, onapriston,e toremifene (Fareaton®), 4 (5)-imidazoles, megestrol acetate (Megase®), exemestane (Aromasin®), anastrozole (Arimidex®), flutamide, nilutamide, bicalutamide, leuprolide, goselerin, SC209, hemiasterlin, muscotoxin A, thailanstatin A, PNU-159682, γ-amanitin, β-amanitin, mensacarcin, luisol A, myotoxin B, isofistularin-3, tomaymycin DM, polyketomycin, piercidin A, SGD-1881, anthramycin (AMC), asparaginase, azathioprine, buthionine sulfoximine (BSO), cytidine arabinoside, cytochalasin B, gramicidin D, mithramycin, palytoxin, plicamycin, enediyene, lexitropsin, echinomycin, netropsin, nocodazole, colcimid, colchicine, combretastatin, cemadotin, discodermolide, T67 (Tularik®), cryptophycin 1, cryptophycin 8, sarcodictyin, pancratistatin, narciclasine, 2-epi-narciclasine, narciprimine, bryostatin, and callystatin.

In the context of the invention the term “immunomodulator” refers to therapeutically active compounds that interact with the immune system, which may for example be useful in the treatment of cancer or autoimmune diseases, optionally as part of combination therapies. Such agents include anti-PD-1 antibody such as pembrolizumab and nivolumab, anti-PD-L1 antibody such as avelumab, anti-CTLA4 antibody such as ipilimumab, cytokine agonists such as proleukin and interferon alfa-2a/interferon alfa-2b, mesalamine, baricitinib, filgotinib, tofacitinib, upadacitinib, ruxolitinib, peficitinib, decemotinib, solcitinib, itacitinib, fostamatinib, and SHR0302.

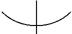

denotes the point of attachment to the adjacent moiety.

### DETAILED DESCRIPTION OF THE INVENTION

The present invention refers to compound having the formula (I)

T-K-A-L-B (I)

The present invention is also directed to a pharmaceutical composition comprising the compound according to claim 1, or a stereoisomer, racemic mixture, pharmaceutically acceptable salt, ester, hydrate, or solvate thereof, and a pharmaceutically acceptable carrier or excipient.

In a further embodiment, the present invention relates to a method of treating, ameliorating, preventing a disorder selected from a neoplastic disorder; atherosclerosis; an autoimmune disorder; an inflammatory disease; a chronic inflammatory autoimmune disease; ischaemia; and reperfusion injury, wherein a therapeutically effective amount of a compound according to claim 1, or a stereoisomer, racemic mixture, pharmaceutically acceptable salt, ester, hydrate, or solvate thereof, is administered to a patient in need thereof.

Precursors of the claimed compound having the formula (I) are compounds having the formulae (II), (III), (IV) and (V).

K′-A-L-B (II)

K′-A-L′ (III)

Q¹-D²-D¹-A-L-B (IV)

K″-A-L′ (V)

Stereoisomers, racemic mixtures, pharmaceutically acceptable salts, esters, hydrates, or solvates of the compounds having the formulae (I), (II), (III), (IV) and (V) are also within the scope of the present invention.
T is a targeting unit that binds to a target biomolecule of interest. The target biomolecule of interest is preferably a cellular biomolecule of a natural (i.e naturally occurring) cell or a cell of a cell line, in particular a cancer cell or cancer cell line. In one embodiment, the target biolmolecule can be selected from a protein, a peptide or a polysaccharide, preferably a peptide or a protein. T is not particularly limited and can be an antibody or an antigen binding fragment. In one embodiment, T is selected from a monoclonal antibody (mAb), polyclonal antibody, antibody fragment, Fab, Fab′, Fab′-SH, Fv, single chain Fv, diabody, linear antibody, bispecific antibody, multispecific antibody, chimeric antibody, humanized antibody, human antibody, fusion protein, nanobody, fusion protein comprising the antigen-binding portion of an antibody and antibody mimetic, preferably T is a monoclonal antibody (mAb). It is understood that the targeting unit comprises one or more binding subunits which are capable of binding to the target molecule. The the antigen can be selected from APO-3, α-fetoprotein, BCMA, carcinoembryogenic antigen, CD2, CD3, CD8, CD11a, CD11b, CD11c, CD18, CD19, CD20, CD22, CD28, CD29, CD30, CD40, CD41, CD49a, CD49b, CD49c, CD49d, CD49e, CD49f, CD70, CD79, CD79b CD90, CD103, CD104, CD152/CTLA-4, CD221, PD-1, ICOS, CD27, CD40, CD95/Fas, CD134/BCMA, EGFR, EpCAM, FAP, HER2, IGF-1 receptor, IL-6, mor antigen CTAA16.88, MUC1, Nectin-4, osteoprotegerin, PDGF-Ra, SMAL7, TACSTD1, tissue factor (TF), TRAIL, vimentin, Apo2/TRAIL-R1, TRAIL-R2, TRAIL-R3, TRAIL-R4, TROP-2, C-type lectin, S-type lectin, and I-type lectin. Examples of T include but are not limited to ABT-806, ABT-874, adalimumab, adecutumumab, aflibercept, afutuzumab, alefacept, alemtuzumab, AMG-53, anetumab, atacicept, atezolizumab, atlizumab, basiliximab, belantamab, belatacept, belimumab, bevacizumab, bivatuzumab, blinatumomab, brentuximab, canakinumab, cantuzumab, catumaxomab, certolizumab, cetuximab, citatuzumab, conatumumab, dacetuzumab, daclizumab, daratumumab, denosumab, dinutuximab, durvalumab, eculizumab, efalizumab, elotuzumab, enfortumab, epratuzumab, etanercept, etaracizumab, farletuzumab, figitumumab, futuximab, gemtuzumab, girentuximab, glembatumumab, golimumab, HuMax CD20, HuMax EGFR, HuMax-CD4, ibritumomab, IDEC-114, IGN101, infliximab, inotuzumab, ipilimumab, iratumumab, isatuximab, labetuzumab, lexatumumab, lintuzumab, lorvotuzumab, loncastuximab, lucatumumab, mapatumumab, matuzumab, mepolizumab, milatuzumab, mogamulizumab, moxetumomab, muromonab-CD3, abciximab, natalizumab, necitumumab, nimotuzumab, nivolumab, ofatumumab, olaratumab, omalizumab, oportuzumab, oregovomab, otelixizumab, palivizumab, panitumumab, pankomab, pembrolizumab, pertuzumab, polatuzumab, pritumumab, ramucirumab, ranibizumab, rilonacept, rituximab, robatumumab, romiplostim, rovalpituzumab, sacituzumab, sibrotuzumab, siltuximab, tacatuzumab, teplizumab, tetulumab, ticilimumab, tigatuzumab, tisotumab, tocilizumab, tositumomab, trastuzumab, tremelimumab, tucotuzumab, ustekinumab, vadastuximab, veltuzumab, vorsetuzumab, votumumab, and zalutumumab. The targeting unit can be linked to K via any atom present in the targeting unit such as C, O, N, S, Se, or P, preferably via O, N, S, more preferably via S.
K is a linker that is composed of —W-D⁴-D³-D²-D¹-, wherein W is bound to T and D¹ is bound to the N atom of A. Thus, the compounds having the formula (I) can be written as follows:

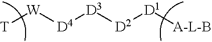

A is a structure of formula (I)

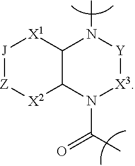

Thus, the compounds having the formula (I) have the formula

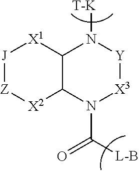

J and Z are independently selected from S or Se such that either J is Se and Z is S, or J is S and Z is Se, or J and Z are both S, preferably J and Z are both S.
X¹ is —(CR^{d}₂)ₘ—, X¹ is preferably —CH₂—.
X² is —(CR^{e}₂)ₙ—, X² is preferably —CH₂—.
X³ is —CR^{f}₂—, X³ is preferably —CH₂—.
Y is —(CR^{g}₂)ₚ—; preferably —(CH₂)— or —(CH₂)—(CH₂)—, more preferably —(CH₂)—.
L is a bond or a self-immolative spacer which is bound to the C(═O) of A. In one embodiment, L is a bond. In another embodiment, L is selected, so that the compound having the formula (I) is selected from:

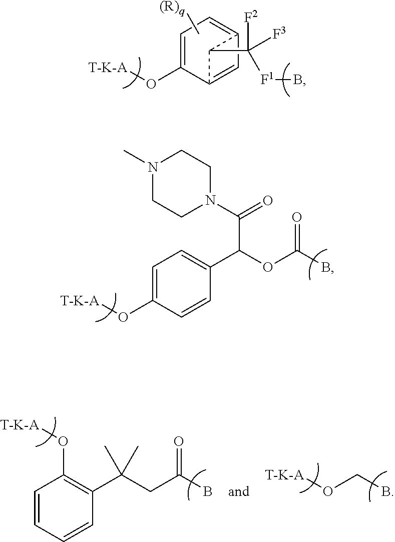

In a further embodiment, L is selected, so that the compound having the formula (I) is selected from:

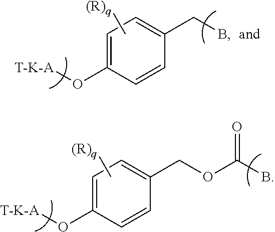

B is —O-A¹, —N(A²)-A³, —S-A⁴, or —O(O)C-A⁵. Consequently, the compounds having the formula (I) have the formula

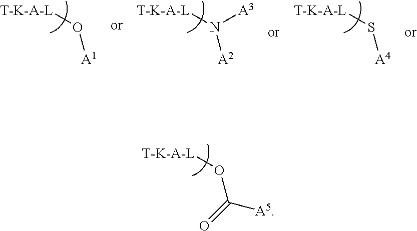

A¹ is selected such that A¹-OH is a therapeutic agent which contains an —OH moiety attached to a carbon, and for which the bond attaching L to that O atom replaces its bond to hydrogen that is present in the agent A¹-OH. A¹-OH is not particularly limited; it contains from 3 to 80 carbon atoms, preferably from 5 to 50 carbon atoms, more preferably 6 to 50 carbon atoms; as well as 1 or more heteroatoms, preferably 1 to 20 heteroatoms, more preferably 1 to 15 heteroatoms, wherein the heteroatoms can be selected from O, N, S, Se, Si, Hal, B or P, preferably selected from O, N, Hal, S, Si or P, more preferably selected from O, N, Hal, S, Si, even more preferably selected from O, N or Hal. Preferably, A¹ can be selected such that A¹-OH is a therapeutic agent where the —OH group is a hydroxyl group that is attached to a carbon which is part of a 5- or 6-membered aromatic or heteroaromatic ring, where this ring can be fused to a further ring or rings, such that the carbon is preferably part of a phenyl, pyridinyl, naphthyl, quinolyl, isoquinolyl, indolyl, pyrrolyl, thiophenyl, pyridinyl, pyrazolyl, pyrimidinyl, furanyl, oxazolyl, thiazolyl, imidazolyl, purinyl, pyrrolo, benzofuranyl, benzothiophenyl, benzothiazolyl, benzoxazolyl, naphthyridinyl, quinoxalyl, benzopyranonyl, benzo[1,3]dioxolyl, benzothiazolyl, 2,3-dihydro-1H-benzo[e]indole, xanthenyl or indolyl ring; more preferably it is part of a phenyl, pyridinyl, benzopyranonyl, naphthyl, benzo[1,3]dioxolyl, 2,3-dihydro-1H-benzo[e]indole, quinolyl or xanthenyl ring; still more preferably, it is part of a phenyl ring; and in all cases the ring can be substituted and can be part of a larger structure which forms the therapeutic agent A¹-OH.
A² and A³ are selected such that A²-NH-A³ is a therapeutic agent which contains an —NH₂ or —NH-moiety, where the N atom is bound to either one or two carbons as indicated, and for which the bond attaching L to that N atom replaces its bond to hydrogen that is present in the agent A²-NH-A³. A² and A³ can be independently selected and are not particularly limited; A²-NH-A³ contains from 3 to 80 carbon atoms, preferably from 5 to 50 carbon atoms, more preferably 6 to 50 carbon atoms; as well as 1 or more heteroatoms, preferably 1 to 20 heteroatoms, more preferably 1 to 15 heteroatoms, wherein the heteroatoms can be selected from O, N, S, Se, Si, Hal, B or P, preferably selected from O, N, Hal, S, Si or P, more preferably selected from O, N, Hal, S, Si, even more preferably selected from O, N or Hal. A² may optionally be H. Preferably, A² and A³ can be selected such that A²-NH-A³ is a therapeutic agent where the nitrogen N is attached to one or two carbons that are part of a 5- or 6-membered aromatic or heteroaromatic ring or rings, and where the ring or rings can optionally be fused to further rings, such that the carbon or carbons are part of a ring or rings that may preferably be phenyl, pyridinyl, naphthyl, quinolyl, isoquinolyl, indolyl, pyrrolyl, thiophenyl, pyridinyl, pyrazolyl, pyrimidinyl, furanyl, oxazolyl, thiazolyl, imidazolyl, purinyl, pyrrolo, benzofuranyl, benzothiophenyl, benzothiazolyl, benzoxazolyl, naphthyridinyl, quinoxalyl, benzopyranonyl, benzo[1,3]dioxolyl, benzothiazolyl, 2,3-dihydro-1H-benzo[e]indole, xanthenyl or indolyl; more preferably they may be phenyl, pyridinyl, benzopyranonyl, naphthyl, benzo[1,3]dioxolyl, 2,3-dihydro-1H-benzo[e]indole, quinolyl or xanthenyl; still more preferably, they may be phenyl; and in all cases the ring or rings can be substituted and can be part of a larger structure which forms the therapeutic
A²-NH-A³.

A² can be hydrogen or any hydrocarbon moiety which has from 1 to 80 carbon atoms, preferably from 1 to 20 carbon atoms, more preferably 1 to 10 carbon atoms, even more preferably 1 to 4 carbon atoms. The hydrocarbon moiety can optionally contain 1 or more heteroatoms, preferably 1 to 10 heteroatoms, more preferably 1 to 5 heteroatoms, wherein the heteroatoms can be selected from O, N, S, Se, Si, Hal, B or P, preferably selected from O, N, Hal, S, Si or P, more preferably selected from O, N, Hal, S, Si, even more preferably selected from O, N or Hal. In one embodiment A² is a C₁₋₄ alkyl, such as methyl.

A³ can be any hydrocarbon moiety which contains from 3 to 80 carbon atoms, preferably from 5 to 50 carbon atoms, more preferably 6 to 50 carbon atoms. The hydrocarbon moiety can optionally contain 1 or more heteroatoms, preferably 1 to 20 heteroatoms, more preferably 1 to 15 heteroatoms, wherein the heteroatoms can be selected from O, N, S, Se, Si, Hal, B or P, preferably selected from O, N, Hal, S, Si or P, more preferably selected from O, N, Hal, S, Si, even more preferably selected from O, N or Hal.
A⁴ is selected such that A⁴-SH is a therapeutic agent which contains an —SH moiety. The definition of A⁴-SH corresponds to the definition of A¹-OH with the exception that an S atom instead of an O atom is present.
A⁵ is selected such that A⁵-COOH is a therapeutic agent which contains an —COOH moiety.

The nature of the therapeutic agent is not particularly limited, as long as it contains an —OH, —NH₂, —NH—, —SH, or —COOH moiety which after removal of the H is suitable for binding to the self-immolative spacer L, or, if L is a bond, to A, via the O, N, or S atom. As can be seen from the schemes below, the O, N or S atom partakes in the reaction when the moiety A of a compound having the formula (I) is cleaved by a disulfide reductase or its effector disulfide protein. The rest of the therapeutic agent is not involved in this reaction, so that a wide variety of therapeutic agents are suitable for use in the present invention.

Examples of suitable therapeutic agents include, but are not limited to therapeutically acceptable DNA-alkylating agents, therapeutically acceptable DNA-intercalating agents or therapeutically acceptable tubulin-inhibiting agents. In one embodiment, therapeutically acceptable agents are preferred, which include: DNA-alkylating agents that include compounds or structurally and functionally related derivatives selected from, but not limited to the following classes: nitrogen mustards or seco-cyclopropabenzaindoles; DNA-intercalating agents that include compounds or structurally and functionally related derivatives selected from, but not limited to the following classes of compounds: anthracyclins or camptothecins; antiproliferative agents that include compounds or structurally and functionally related derivatives selected from, but not limited to the following classes: monomethyl auristatins, the Amaryllidaceae alkaloids; or any compounds derived by substitution of the same.

Examples of therapeutic agents include, but are not limited to, agents for treating, ameliorating, or preventing a neoplastic disorder, an inflammatory disorder (e.g. a non-steroidal anti-inflammatory drug (NSAID), a disease-modifying anti-rheumatic drug (DMARD), or mesalamine), atherosclerosis; an autoimmune disorder; a chronic inflammatory autoimmune disorder; ischaemia; and reperfusion injury as well as kinase inhibitors.

The therapeutic agent A¹-OH, A²-NH-A³, A⁴-SH, or A⁵-C(O) OH is not specifically limited and can be, for instance, selected from a therapeutically acceptable cytotoxic, cytostatic, or immunosuppressive agent. In one embodiment, the therapeutic agent A¹-OH, A²-NH-A³, A⁴-SH, or A⁵-C(O) OH can be selected from DNA-alkylating agents, DNA-intercalating agents, DNA replication inhibitors, tubulin-inhibiting antimitotics, bifunctional degraders, antibiotics, immunomodulators, kinase inhibitors. In a further embodiment, the therapeutic agent A¹-OH, A²-NH-A³, A⁴-SH, or A⁵-C(O) OH can be selected from topoisomerase inhibitors, duocarmycins, pyrrolobenzodiazepines, trioxacarcins, nitrogen mustards, calicheamycins, mitomycins, doxorubicin derivatives, toxoids, auristatins, tubulysins, folic acid analogues, nitrosoureas, pyrimidines, tamoxifens, androgens, maytansinoids, aziridines, methylamelamines, platinum complexes, bifunctional PROTAC degraders, and molecular glue degraders. Illustrative examples of the therapeutic agent A¹-OH, A²-NH-A³, A⁴-SH, or A⁵-C(O) OH include camptothecin, topotecan, NH₂-bicyclo[1.1.1]pentane-7-MAD-MDCPT, deruxtecan (Dxd), exatecan, Gly-cyclopropane-exatecan (HY-13631), 10-hydroxycamptothecin, 10-hydroxybelotecan, belotecan, 10-hydroxygimatecan, gimatecan, CKD-602, BNP-1350, sinotecan, 7-ethyl-10-hydroxy-camptothecin (SN-38), 10-hydroxy-20-acetoxy-camptothecin, irinotecan (CPT-11), RFS 2000, duocarmycin A, duocarmycin SA, seco-duocarmycin, duocarmycin DM, duocarmycin GA, duocarmycin MB, duocarmycin MA, seco-DUBA, duocarmycin DC1, duocarmycin DC4, CBI-TMI, DC0-NH₂, CC-1065, adozelesin, KW-2189, carzelesin, bizelesin, 5-hydroxy-seco-cyclopropabenzaindoles, 5-hydroxy-seco-(2-methyl-cyclopropa)benzaindoles, 5-hydroxy-seco-cyclopropa-methoxybenzaindoles, 5-amino-seco-cyclopropabenzaindoles, C16-modified trioxacarcins, DC-45-A1, DC-45-A2, trioxacarcin A, trioxacarcin C, trioxacarcin D, LL-D49194α1, pyrrolobenzodiazepine, chlorambucil, 4-(bis(2-chloroethyl)amino)phenol, 4-(bis(2-bromoethyl)amino)phenol, 4-((2-chloroethyl-2′-mesylethyl)amino)phenol, 4-(bis(2-mesylethyl)amino)phenol, chlornaphazine, cyclophosphamide, estramustine, ifosfamide, mechlorethamine, mechlorethamine oxide, melphalan, novembichin, phenesterin, prednimustine, trofosfamide, uracil mustard, calicheamycin, calicheamycin α1, calicheamycin β1, calicheamycin γ1,calicheamycin ω1, calicheamycin T, calicheamycin δ1, calicheamycin ε1, dynemicin, dynemicin A, carabicin, carzinophillin, actinomycin, antrmycin, bleomycin, cactinomycin, carninomycin, chromomycin, dactinomycin, marcellomycin, mitomycin C, mycophenolic acid, nogalamycin, olivomycin, peplomycin, potfiromycin, puromycin, quelamycin, clodronate, esperamicin, neocarzinostatin, aclacinomysin, azaserine, daunorubicin, detorubicin, 6-diazo-5-oxo-L-norleucine, doxorubicin (Adrlimycin@), morpholino-doxorubicin, cyanomorpholino-doxorubicin, 2-pyrrolino-doxorubicin, deoxydoxorubicin, DMEA-PNU-159682, doxorubicin-N,O-acetal, pirarubicin, epirubicin, esorubicin, rodorubicin, tubercidin, zorubicin, daunomycin, streptomigrin, streptozotocin, ubenimex, valrubicin, denopterin, methotrexate, pemetrexed, raltitrexed, pteropterin, trimetrexate, fludarabine, 6-mercaptopurine, thiamiprine, thiguanine, folinic acid, aceglatone, aldophophamide glycoside, aminolevulinic acid, eniluracil, amsacrine, bestrabucil, bisantrene, edatrexate, defofamine, demecolcine, diaziquone, elfornithine, elliptinium acetate, etoglucid, hydroxyurea, lentinan, lonidainine, carmustine, chlorozotocin, fotemustine, lomustine (CCNU), nimustine, ranimnustine, ancitabine, azacytidine, 6-azauridine, carmofur, cytarabine, dideoxyuridine, doxifluridine, enocitabine, gemcitabine, 6-thioguanine, troxacitabine, 5-azacytidine, 5-fluordeoxyuridine, cytarabine, cladribine, clofarabine, decitabine, floxuridine, nelarabine, tegafur, tioguanine, calusterone, dromostanolone, propionate, epitiostanol, mepitiostane, testolactone, aminoglutethimide, mitotane, trilostane, maytansinoids, maytansine DM1, maytansine DM3, N-Me-L-Ala-maytansinol, ansamitocin, mitoguazone, mitoxantrone, mopidanmol, nitraerine, pentostatin, phenamet, losoxantrone, 2-ethylhydrazide, procarbazine, razoxane, rhizoxin, sizofiran, spirogermanium, tenuazonic acid, triaziquone, trichothecene, T-2 toxin, verracurin A, roridin A, anguidine, urethane, dacarbazine, mannomustine, mitobronitol, mitolactol, pipobroman, gacytosine, arabinoside, cyclophosphamide (Cytoxan®), busulfan, improsulfan, piposulfan, benzodopa, meturedopa, uredopa, carboquone, altretamine, triethylenemelamine, triethylenephosphoramide, triethylenethiophosphoramide, trimethylolomelamine, bullatacin, bullatacinone, ribociclib, erlotinib (Tarceva®), bortezomib (Velcade®), fulvestrant (Faslodex®), letrozole (Femara®), imatinib (Gleevec®), sutent (SU11248), PTK787/ZK 222584, oxaliplatin (Eloxatin®), cisplatin, carboplatin, 5-fluorouracil (5-FU), capecitabine, leucovorin, rapamycin (Rapamune®), lapatinib (Tykerb®), lonafamib, sorafenib (Bay43-9006), gefitinib (Iressa®), baricitinib, filgotinib, tofacitinib, upadacitinib, ruxolitinib, peficitinib, decemotinib, solcitinib, itacitinib, fostamatinib, Ivarmacitinib (SHR0302), AG1478, AG1571, thiotepa, MT-802, SJF620, P131, L181, CJH-005-067, DD-04-015, DD-03-171, MZ-1, SNIPER (BRD4)-1, 1-208 (Kymera), dBET1, BETd-260/ZBC260, A1874, QCA570, ARV-771, ARV-825, ARV-110, ARV-471, DT2216, MS40, SJF-α, SJF-δ, 753b, SIM1, I-8, DP-C-1, CFT7455, eragidomide (CC-90009), NVP-DKY709, E7820, (R)-CR8, TMX-4116, NRX-252114, BI-3802, mezigdomide (CC-92480), thalidomide, lenalidomide, pomalidomide, avodomide (CC-122), 5-hydroxythalidomide, FPFT-2216, CC-647, CC-3060, iberdomide, CC-885, ZXH-1-161, indisulfam, paclitaxel (Taxol®), docetaxel (Taxotere®), cabazitaxel, epothilone A, epothilone B, epothilone, etoposide, teniposide, eleutherobin, vinblastine, vincristine, vindesine, vinorelbine, novantrone, edatrexate, aminopterin, xeloda, ibandronate, difluoromethylornithine (DFMO), retinoic acid, capecitabin, monomethyl auristatin E (MMAE), monomethyl auristatin F (MMAF), monomethyl auristatin D (MMAD), auristatin, PF-06380101 (auristatin-0101), duostatin, dolastatins, dolastatin 10, dolastatin 15, symplostatin 1, symplostatin 3, symplostatin 4, auristatin PE, zinostatin, spongistatin, tubulysin IM-3, tubulysin A, tubulysin B, tubulysin C, tubulysin G, tubulysin I, tamoxifen (Nolvadex®), raloxifene, droloxifene, 4-hydroxytamoxifen, trioxifene, keoxifene, LY117018, onapriston, e toremifene (Fareaton®), 4 (5)-imidazoles, megestrol acetate (Megase®), exemestane (Aromasin®), anastrozole (Arimidex®), flutamide, nilutamide, bicalutamide, leuprolide, goselerin, SC209, hemiasterlin, muscotoxin A, thailanstatin A, PNU-159682, γ-amanitin, β-amanitin, mensacarcin, luisol A, myotoxin B, isofistularin-3, tomaymycin DM, polyketomycin, piercidin A, SGD-1881, anthramycin (AMC), asparaginase, azathioprine, buthionine sulfoximine (BSO), cytidine arabinoside, cytochalasin B, gramicidin D, mithramycin, palytoxin, plicamycin, enediyene, lexitropsin, echinomycin, netropsin, nocodazole, colcimid, colchicine, combretastatin, cemadotin, discodermolide, T67 (Tularik®), cryptophycin 1, cryptophycin 8, sarcodictyin, pancratistatin, narciclasine, 2-epi-narciclasine, narciprimine, bryostatin, and callystatin.
D¹ is selected from —CH₂—, —CH═CH—, —C≡C—, —C(O)—, —C(O)—O—, —C(O)—NH—, —C(O)—S—, —S(O)—, —S(O)₂—, —P(O)(O-E²)-, —S(O)₂—NH—, —P(O)(O-E²)-O—, and —P(O)(O-E²)-NH—, preferably —CH₂—, —C(O)—, —C(O)—O—, —C(O)—NH—, and —S(O)₂—NH—.
D² is either a bond or a spacer. D² is present to provide distance between the targeting unit T-W and the linker-payload moiety D¹-A-L-B. In certain embodiments, the spacer D² is selected from: alkyl, alkenyl, alkynyl, aryl, cycloalkyl, heteroaryl, heterocyclic, or heteroatoms, and combinations thereof. The spacer can be homogenous or heterogenous in its atom content (e.g., spacers containing carbon atoms or spacers containing carbon atoms as well as one or more heteroatoms). Preferably, the spacer contains 1 to 50 carbon atoms and 0-30 heteroatoms selected from oxygen, nitrogen and sulfur. The spacer may also be chiral or achiral, linear, branched or cyclic. In certain embodiments, D² is a spacer selected from polyalkylene glycol, alkylene, alkenylene, alkynylene, polyamine, and a polypeptide comprised of the proteinogenic amino acids. The spacer may comprise 1-80 single units that may also be mixed flexibly between these classes. In certain embodiments, D² is selected from —(CH₂)₁₋₁₆₀— or —(CH₂—CH₂—O)₁₋₈₀—, preferably from —(CH₂)₁₋₃₀— or —(CH₂—CH₂—O)₁₋₃₀—.
D³ is a bond or an adapting unit. The adapting unit is not particularly limited and can be any moiety which is suitable for connecting D² and D⁴. In certain embodiments -D³- is an adapting unit that results from reaction of two independently chosen groups -Q¹ and
—Q², that are found on the side of the targeting unit (T-W-D⁴-Q²) and on the side of the payload (Q¹-D²-D¹-A-L-B) by using any method known in the art. In some embodiments D³ is therefore chosen from the following expanded chemical structures, with grey ovals independently representing either D² or D⁴, although it is not limited to these structures:

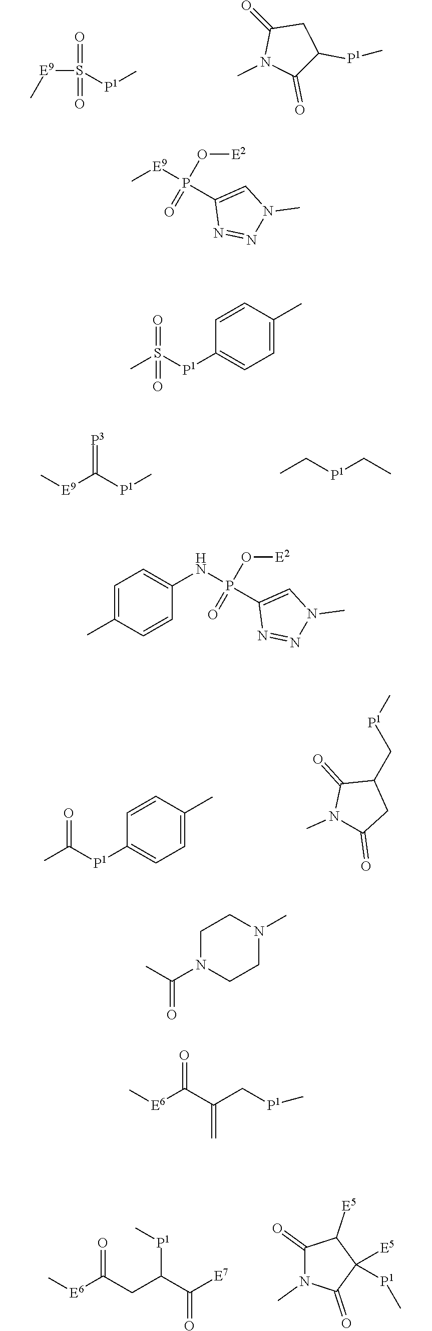

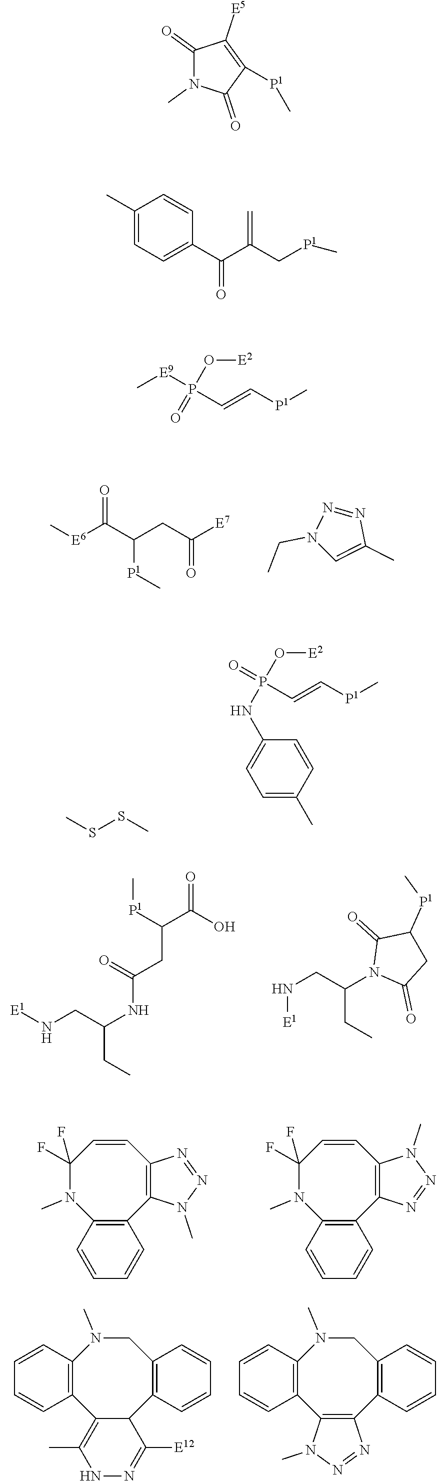

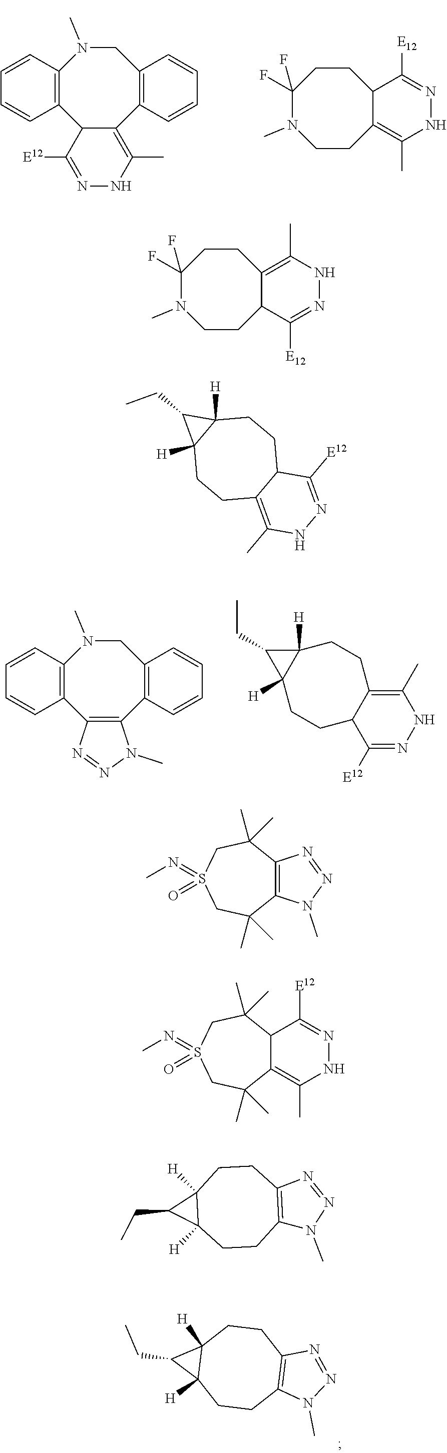

wherein either the dark grey oval represents D² and the light grey oval represents D⁴, or the light grey oval represents D² and the dark grey oval represents D⁴.
D⁴ is either a bond or a spacer. D⁴ is present to provide distance between the targeting unit T-W and the linker-payload moiety D¹-A-L-B. In certain embodiments, the spacers selected from: optionally substituted alkyl, alkenyl, alkynyl, aryl, cycloalkyl, heteroaryl, heterocyclic, or heteroatoms, and combinations thereof. The spacer can be homogenous or heterogenous in its atom content (e.g., spacers containing carbon atoms or spacers containing carbon atoms as well as one or more heteroatoms). Preferably, the spacer contains 1 to 50 carbon atoms and 0-30 heteroatoms selected from oxygen, nitrogen and sulfur. The spacer may also be chiral or achiral, linear, branched or cyclic. In certain embodiments, D⁴ is a spacer selected from polyalkylene glycol, alkylene, alkenylene, alkynylene, polyamine, and a polypeptide comprised of the proteinogenic amino acids. The spacer may comprise 1-80 single units that may also be mixed flexibly between these classes. In certain embodiments, D⁴ is selected from —(CH₂)₁₋₁₆₀— or —(CH₂—CH₂—O)₁₋₈₀—, preferably from —(CH₂)₁₋₃₀— or —(CH₂—CH₂—O)₁₋₃₀—.
E¹ is selected from —H, —C₁₋₄-alkyl, —C(O)-Me, —C(O)—O—C₁₋₄-alkyl, —C(O)—NH—C₁₋₄-alkyl, and —S(O)₂—C₁₋₄-alkyl.
E² is selected from —H, —C₁₋₄-alkyl, —C₁₋₄-alkyl-SO₃H, —C₁₋₄-alkyl-OPO₃H₂; —C(O)-Me, —C(O)—O—C₁₋₄-alkyl, —C(O)—NH—C₁₋₄-alkyl, —S(O)₂—C₁₋₄-alkyl, —(CH₂—CH₂—O)₁₋₈₀—CH₂—CH₂—OH, and —CH₂—CH₂—N(CH₂—CH₂—OH)₂.
E³ is selected from —C≡C-E¹¹, and —CH═CH-(E¹¹).
E⁴ is selected from —H, —C₁₋₄-alkyl, and —O—C₁₋₄-alkyl.
E⁵ is selected from —F, —Cl, —Br, —I, —OMs, and —OTs.
E⁶ is selected from —O—, —NH—, and —S—.
E⁷ is selected from an aromatic or heteroaromatic residue, preferably -Ph.
E⁸ is selected from —F, —Cl, —Br, —I, —OMs, —OTs, —OTf, —C₁₋₄-alkyl, —H, —O—C₁₋₄-alkyl, —(CH₂—CH₂—O)₁₋₈₀—CH₂—CH₂—OH, —CH₂—CH₂—N(CH₂—CH₂—OH)₂.
E⁹ is selected from —O—, —NH—, —N(C₁₋₄-alkyl)-, —CH₂—, and -1,2,3-triazolyl-.
E¹⁰ is selected from —F, —Cl, —Br, —I, —OMs, —OTs, —C≡C-E¹¹, and —CH═CH-(E¹¹).
E¹¹ is selected from —H, —C₁₋₄-alkyl, —NH₂, —NH—C₁₋₄-alkyl, —NH(C₁₋₄-alkyl)₂, and —O—C₁₋₄-alkyl.
E¹² is selected from —H, -Me, -Ph, -2-pyridinyl, and -5-pyrimidinyl.
—F¹—B is selected from

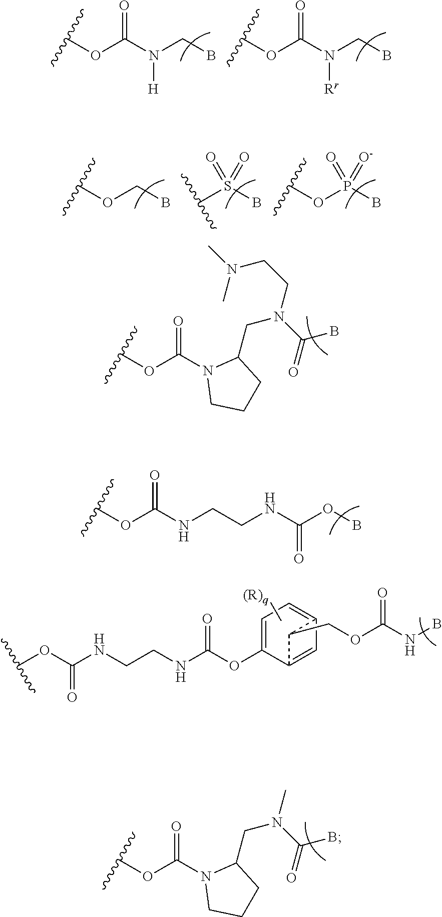

F² is selected from —H, —CF₃, and —C₁₋₄-alkyl.
F³ is selected from —H, —CF₃, and —C₁₋₄-alkyl.
G¹ is selected from —NH—, —O—, —S—, and —Se—.
G² is selected from —CH═C(E¹¹)—, and —CH₂—CH(E¹¹)—.
G³ is selected from —NH—, —O—, —S—, —Se—, —CH═C(E¹¹)—, and —CH₂—CH(E¹¹)—.
K′ is represented by W′-D⁴-D³-D²-D¹-. Thus, the compound having the formula (II) can be written as follows:

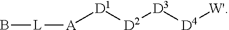

K″ is represented by Q¹-D²-D¹-. Consequently, the compound having the formula (IV) can be written as

Q¹-D²-D¹-A-L-B.
The compound having the formula (V) can be written as

Q¹-D²-D¹-A-L′.
L′ is a leaving group or an activated self-immolative spacer unit. In some embodiments L′ is selected such that the compound having the formula (III) is:

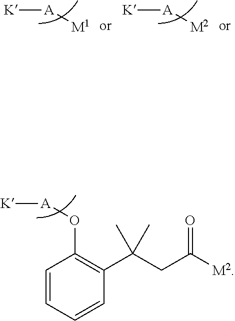

In another embodiment, L′ is selected such that the compound having the formula (III) is:

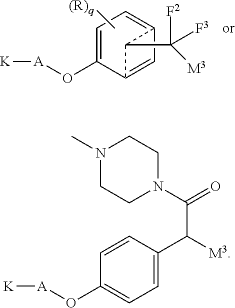

M¹ is a leaving group which is selected from —F, —Cl, —Br, —I, —OMs, —OTs, —OTf, and —CN.
M² is an activated self-immolated spacer selected from

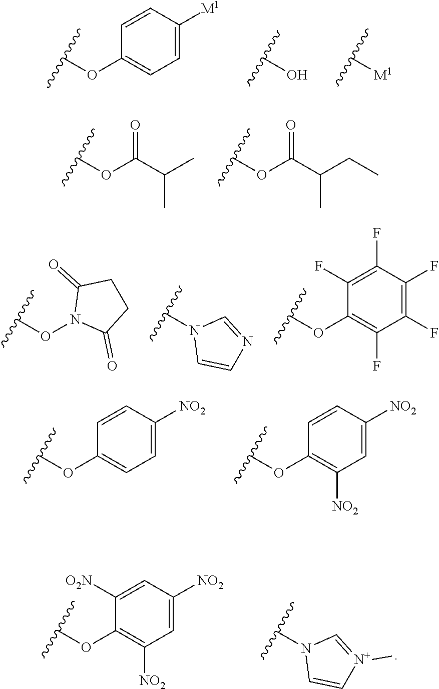

M³ is an activated self-immolated spacer selected from

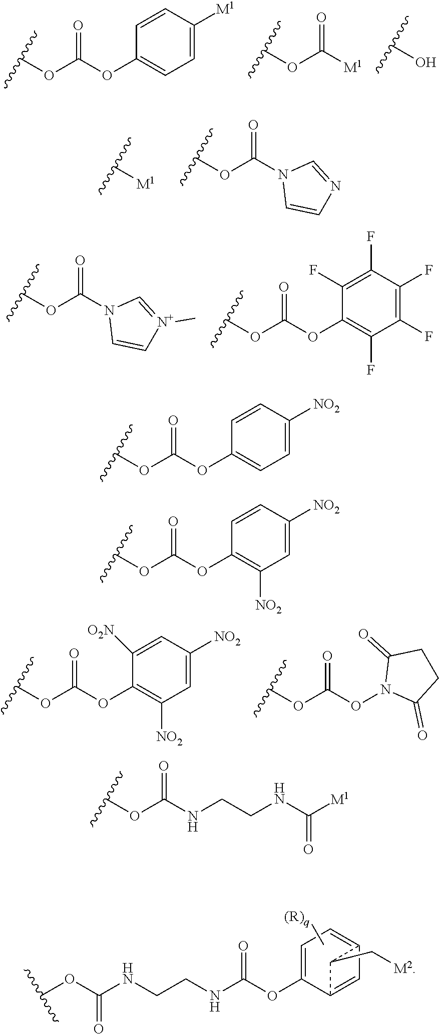

m is 0, 1 or 2; preferably m is 1.
n is 1 or 2, preferably n is 1; provided that m+n is 2 or 3.
p is 1, or 2, preferably p is 1.
P¹ is selected from —O—, —NH—, —N(E²)-, —S—, and —Se—.
P² is selected from —OH, —NH₂, —NH(E²), —SH, and —SeH.
P³ is selected from ═O, ═NH, ═N(E²), ═S, and ═Se.
q is 0, 1, 2, 3 or 4, preferably q is 0 or 1.
Q¹ is a chemical functionalization handle that may be any functional group suitable for assembly of compounds of the invention. Q¹ is not particularly limited. In some embodiments, Q¹ is selected from —CO₂H, —NHFmoc, —NHBoc, —NHCbz, —NHBn, —CCH, —CHCH₂, —CHO, —NH₂, —N₃, —OH, —Br, —I, —Cl, —OTf, —OTs, —OMs, —CO₂tBu, —OAc, —OTFA, —CO₂Bn, —CO₂Ac, —CO₂Piv, —CN, —SH, —SAc, —SMs, and —STs. In a further embodiment, Q¹ can be chosen such that the substructure Q¹-D²-D¹-A-L-B (compound having the formula (IV) is chosen from the following:

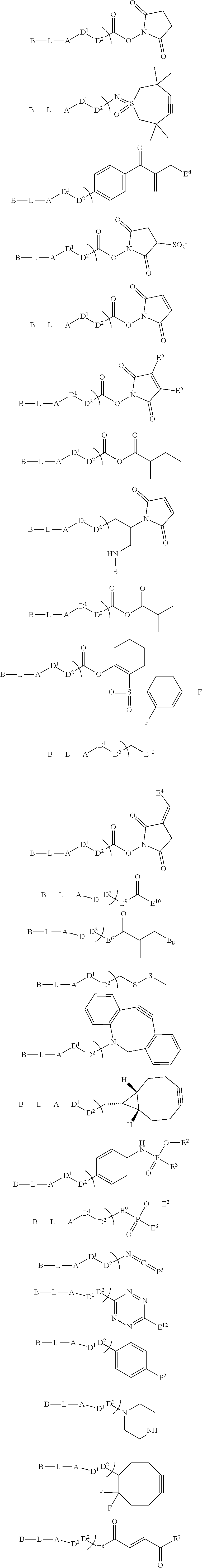

Q² is a chemical functionalization handle that may be any functional group suitable for assembly of compounds of the invention. Q² is not particularly limited. In some embodiments, Q² is selected from —CO₂H, —NHFmoc, —NHBoc, —NHCbz, —NHBn, —CCH, —CHCH₂, —CHO, —NH₂, —N₃, —OH, —Br, —I, —Cl, —OTf, —OTs, —OMs, —CO₂tBu, —OAc, —OTFA, —CO₂Bn, —CO₂Ac, —CO₂Piv, —CN, —SH, —SAc, —SMs, —STs. In a further embodiment, Q² can be chosen such that the substructure T-W-D⁴-Q² is chosen from the following:

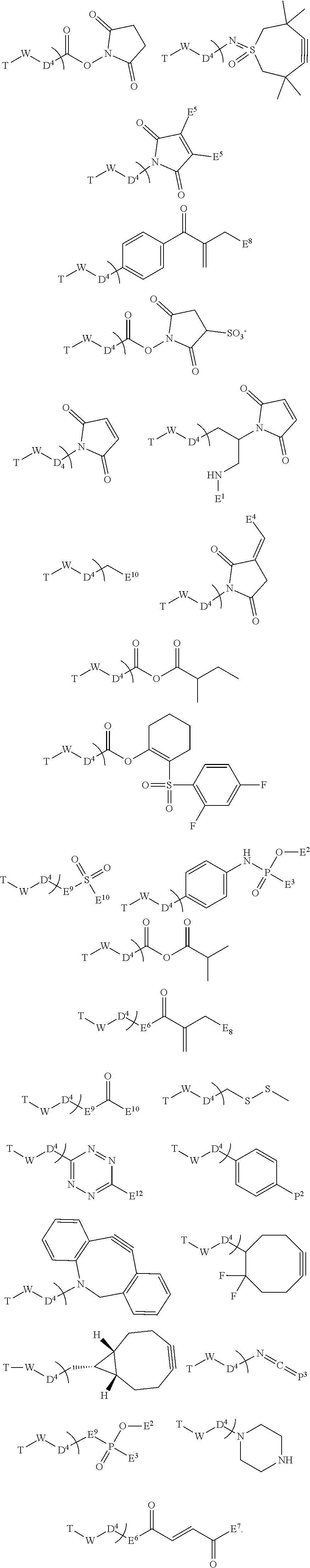

R is independently selected from halogen, —O(R^{r}), —N(R^{s})(R^{t}), —NO₂, —CN, and a heterocyclic group selected from azetidinyl, pyrrolidinyl, piperidinyl and morpholino, wherein the heterocyclic group is attached to the phenyl ring via the N atom. Preferably, group R is a halogen (such as F or Cl) in ortho position relative to the bond to A; also preferably, group R is —O(R^{r}) in ortho position relative to the bond to B.
R^{d} is independently selected from —H and —C₁₋₄-alkyl, preferably H.
R^{e} is independently selected from —H and —C₁₋₄-alkyl, preferably H.
R^{f} is independently selected from —H and —C₁₋₄-alkyl, preferably H.
R^{g} is independently selected from —H and —C₁₋₄-alkyl, preferably H.
R^{r} is selected from —H, —C₁₋₄-alkyl and —(C₂₋₄-alkylene)-O—(C₁₋₄-alkyl), preferably —C₁₋₄-alkyl, more preferably R^{r} is methyl.
R^{s} is selected from —H, —C(O)—C₁₋₄-alkyl and —C₁₋₄-alkyl, preferably —H or —C₁₋₄-alkyl, more preferably —H or —CH₃.
R^{t} is selected from —H, —C(O)—C₁₋₄-alkyl and —C₁₋₄-alkyl, preferably —H or —C₁₋₄-alkyl, more preferably —H or —CH₃.
W is a bioconjugation attachment that results from reaction of a functional group with the targeting unit T. For instance, W is a bioconjugation attachment that may result from reaction of any functional group suitable for conjugating the drug-containing small molecule linker-payload precursor W′-D⁴-D³-D²-D¹-A-L-B with suitable functional groups, e.g., present in amino acid side chains, in the targeting unit T by using any method known in the art. In some embodiments W is selected from but not limited to structures such that formula (I) is

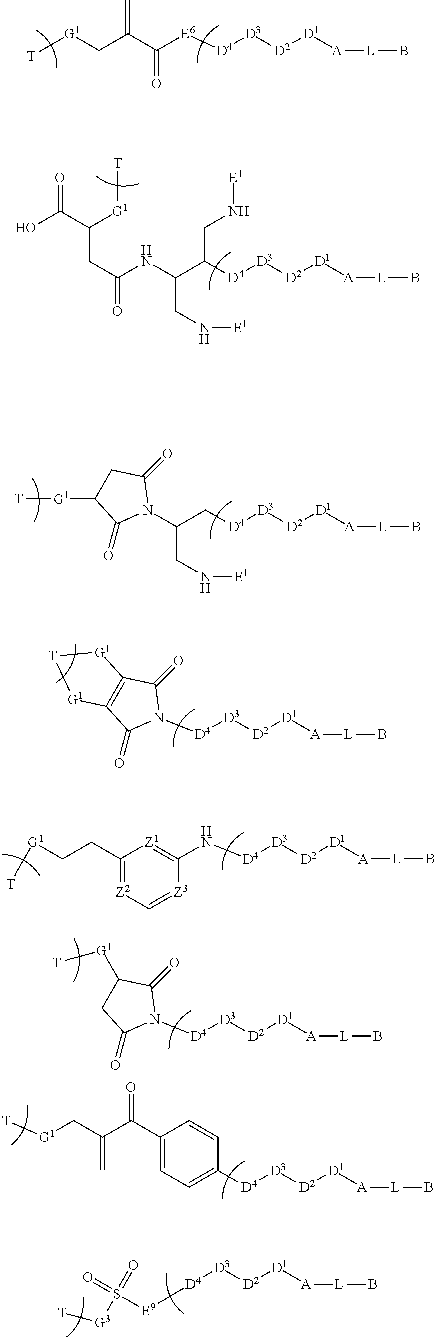

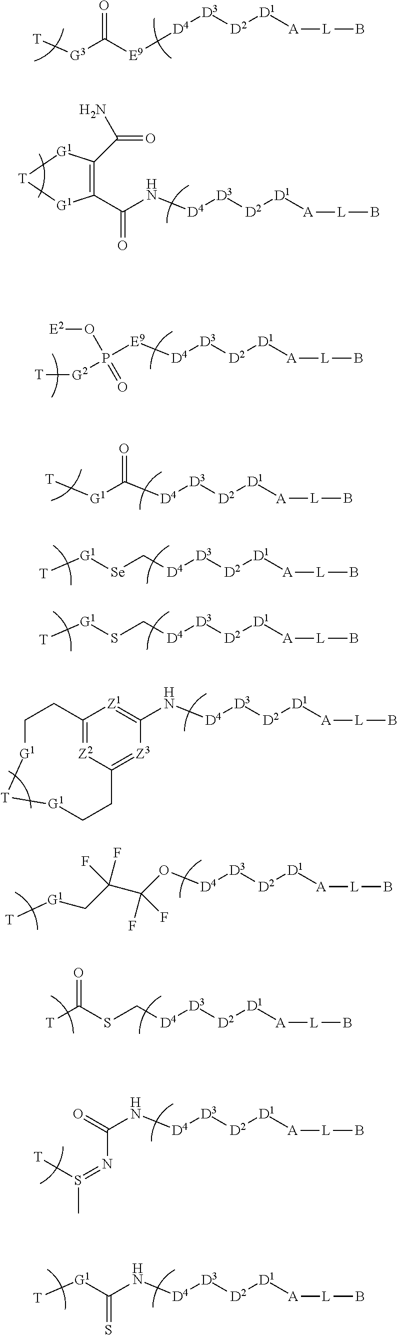

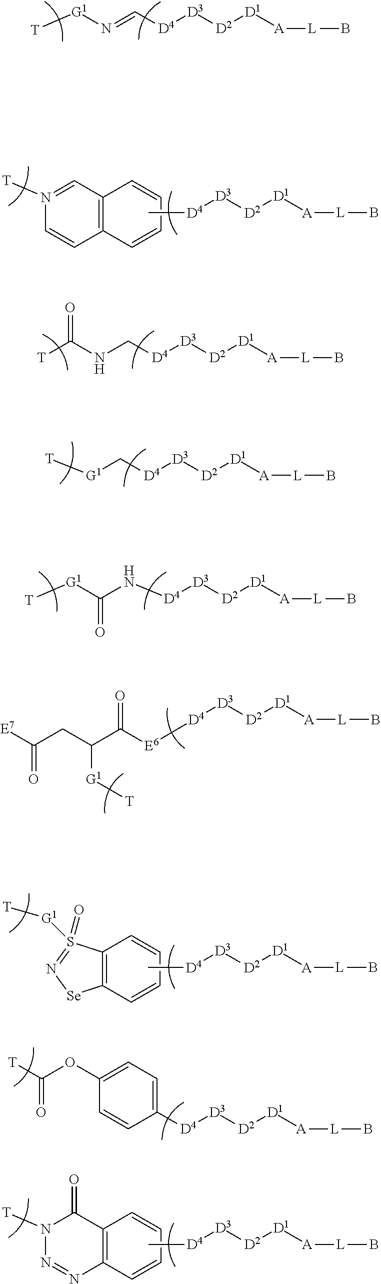

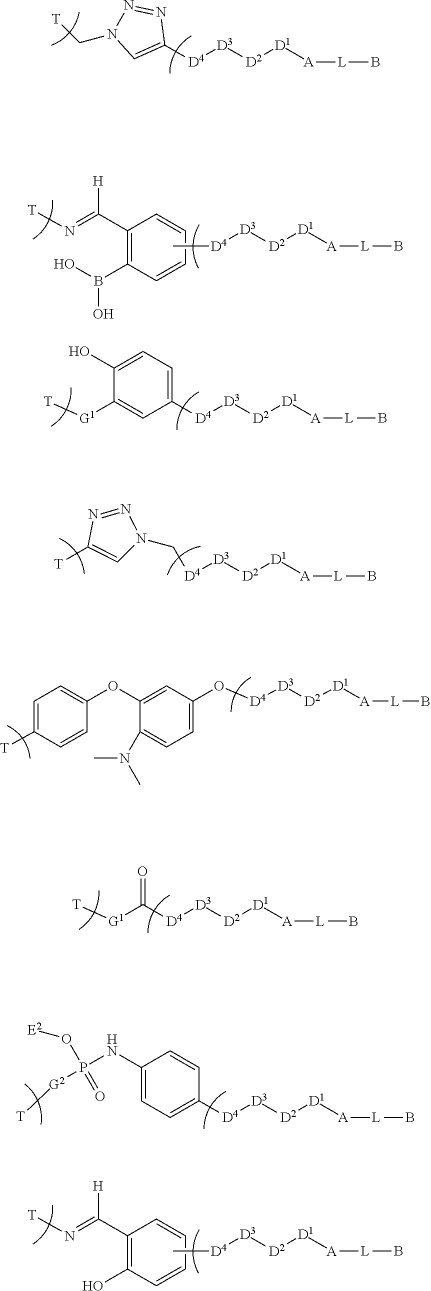

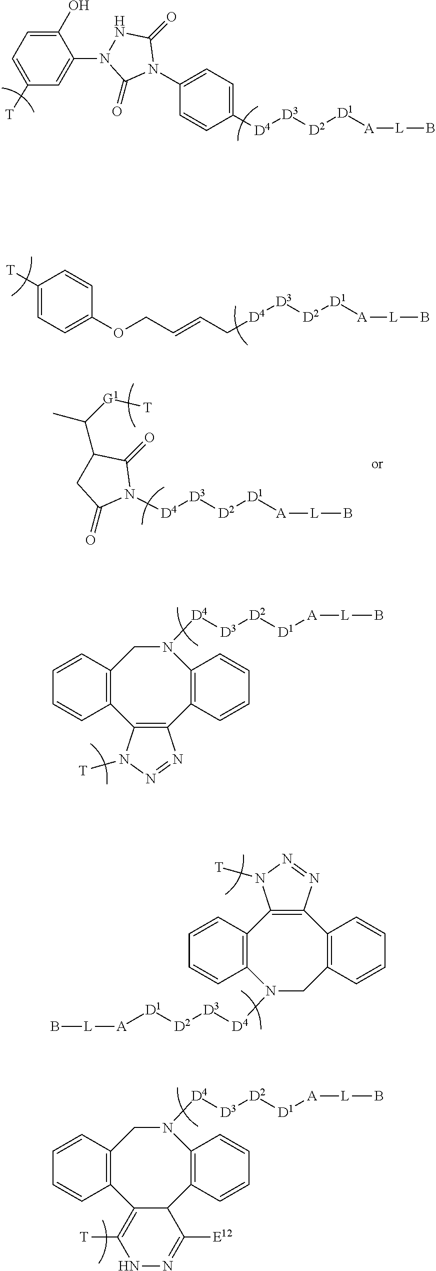

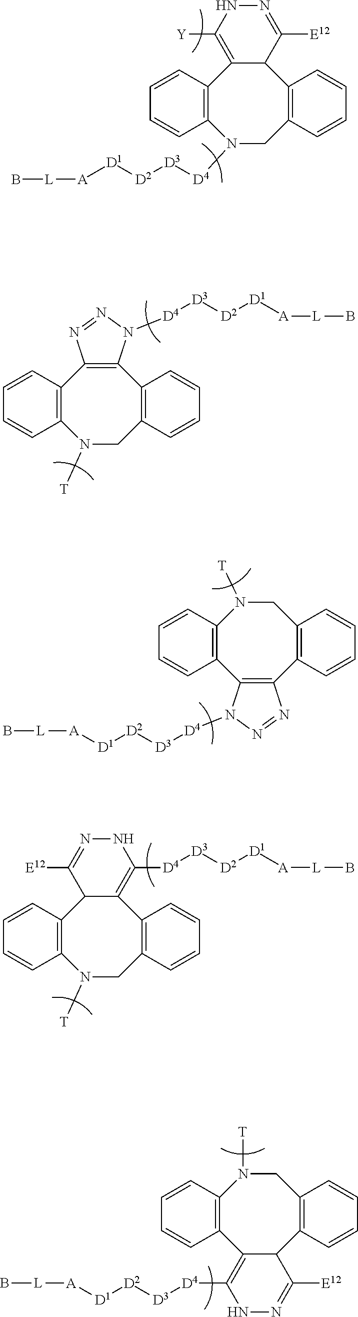

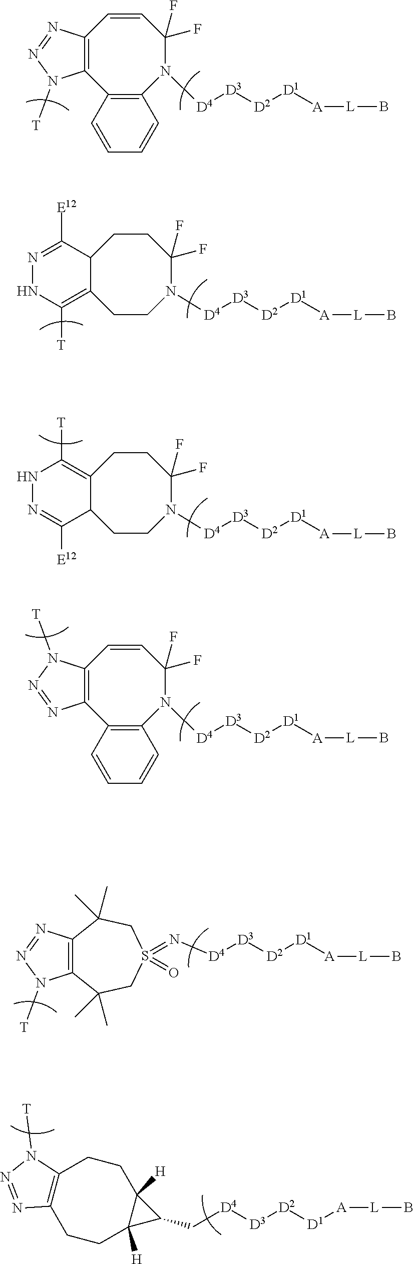

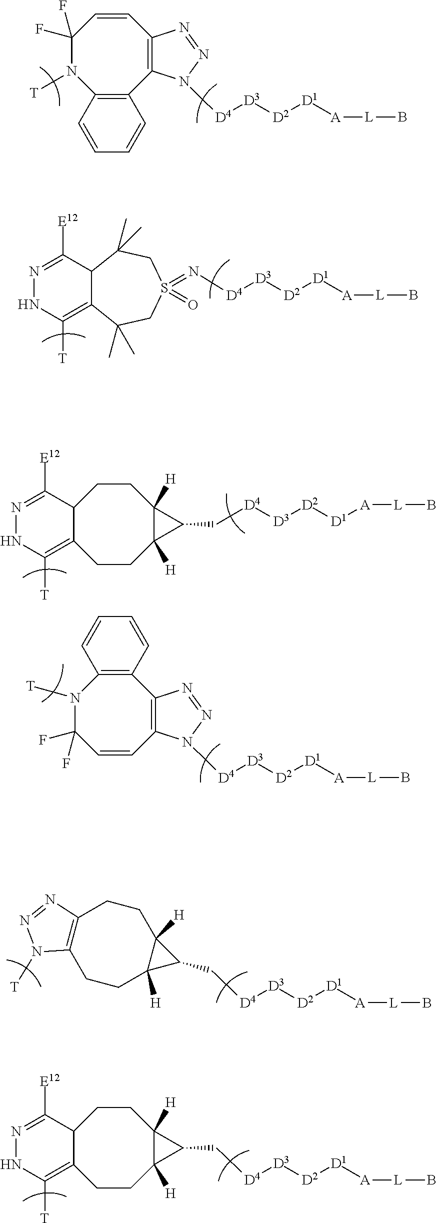

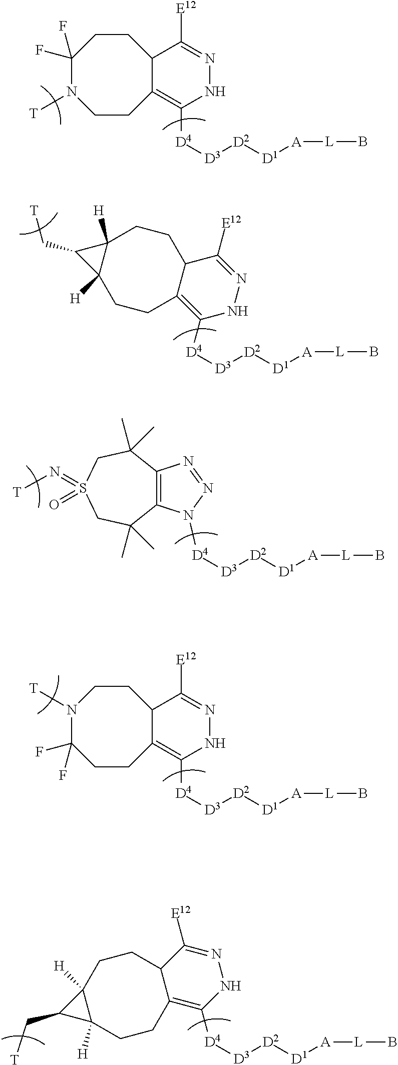

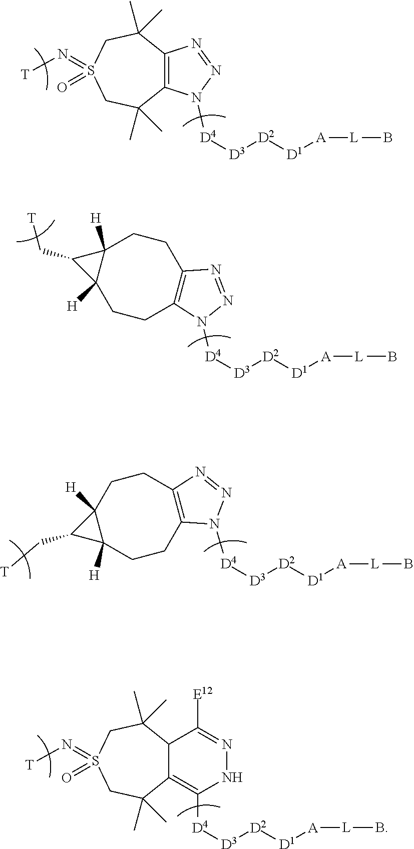

W′ is a functional group suitable for binding to an amino acid side chain in the targeting unit T. For instance, W′ can be selected from any functional group suitable for conjugating the drug-containing small molecule linker-payload precursor (W′-D⁴-D³-D²-D¹-A-L-B) with suitable functional groups, e.g., present in amino acid side chains, in the targeting unit T by using any method known in the art. In certain embodiments it is a reactive functional group such that the substructure W-D⁴-D³-D²-D¹-A-L-B (compound having the formula (II)) is represented by:

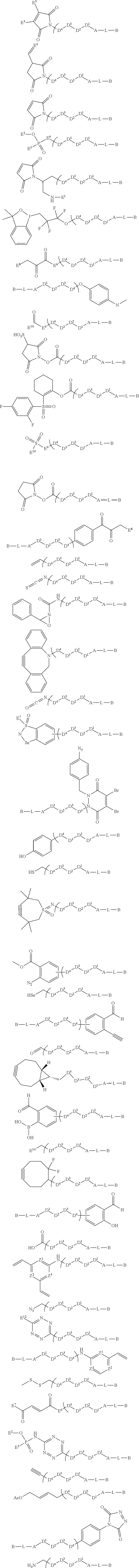

Z¹, Z², Z³ are independently selected from CH, and N.

In a preferred embodiment, the compound having the formula (I) is selected from

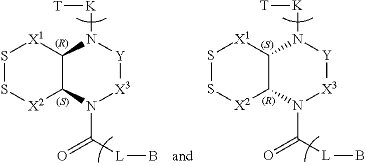

and mixtures thereof.

It is understood that all combinations of the above definitions and preferred definitions are envisaged by the present inventors.

Illustrative embodiments of the compound having the formula (I) include

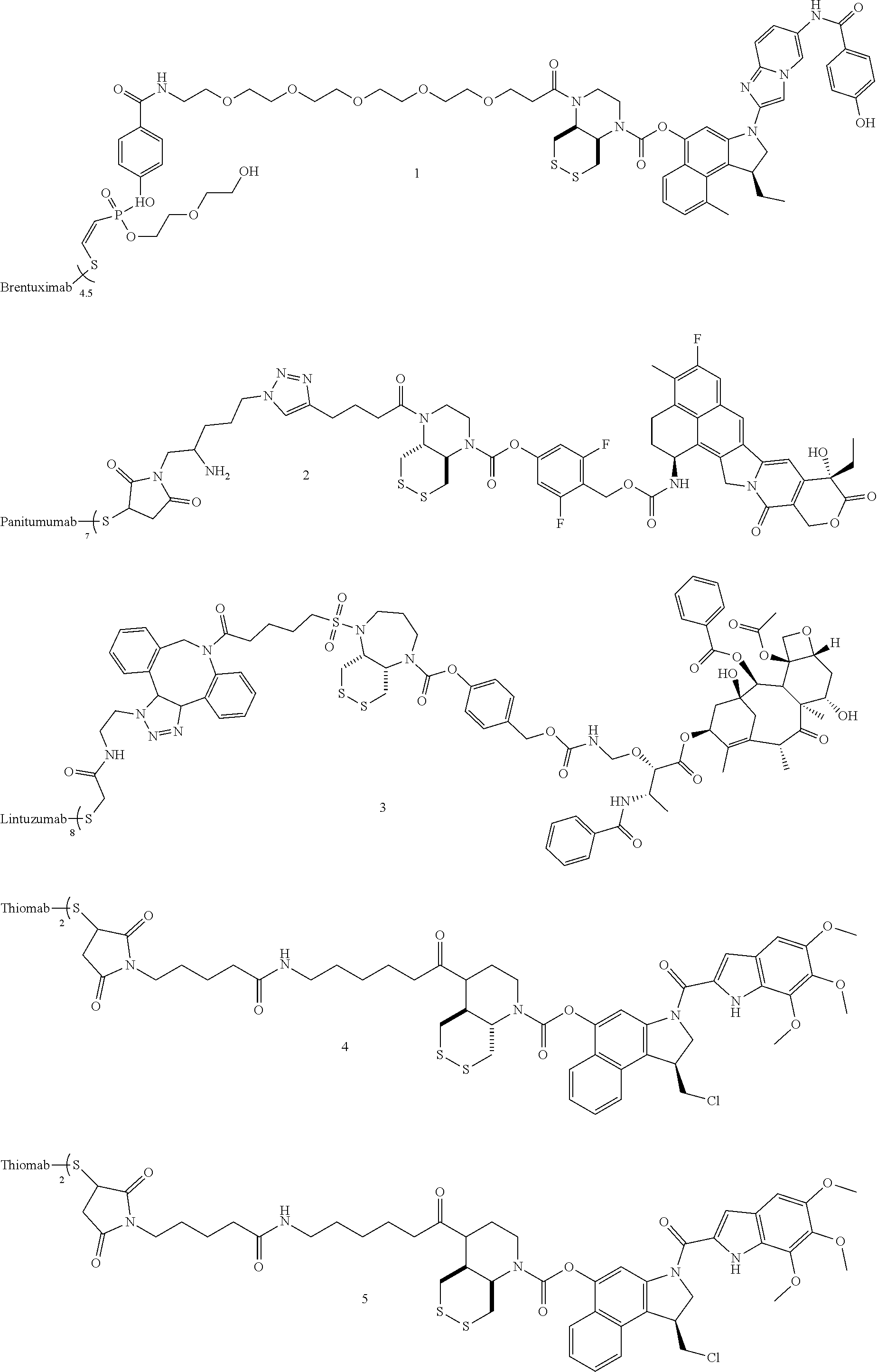

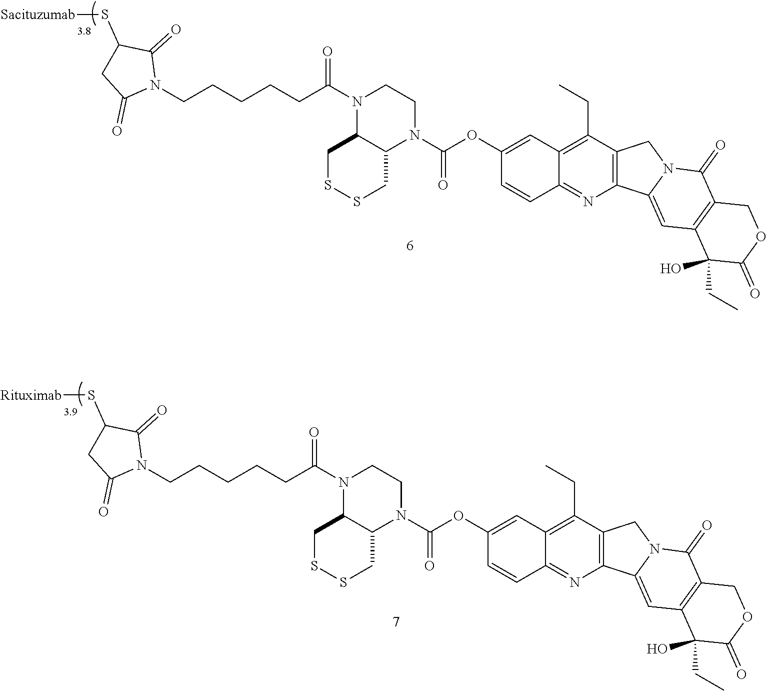

It is understood that other targeting units than Brentuximab, Panitumumab, Lintuzumab, Thiomab, Sacituzumab, and Rituximab can also be employed and that the Drug-To-Antibody ratios can also vary as applicable.

Illustrative embodiments of the compound having the formula (II) include

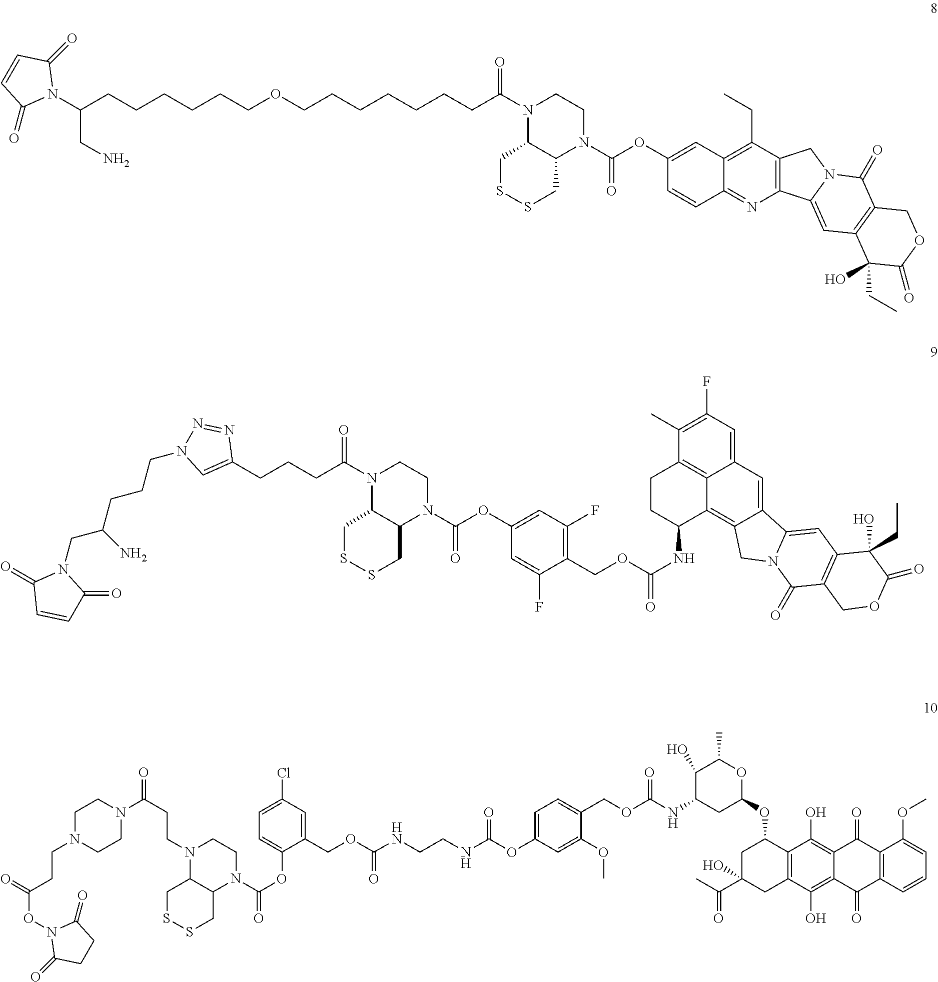

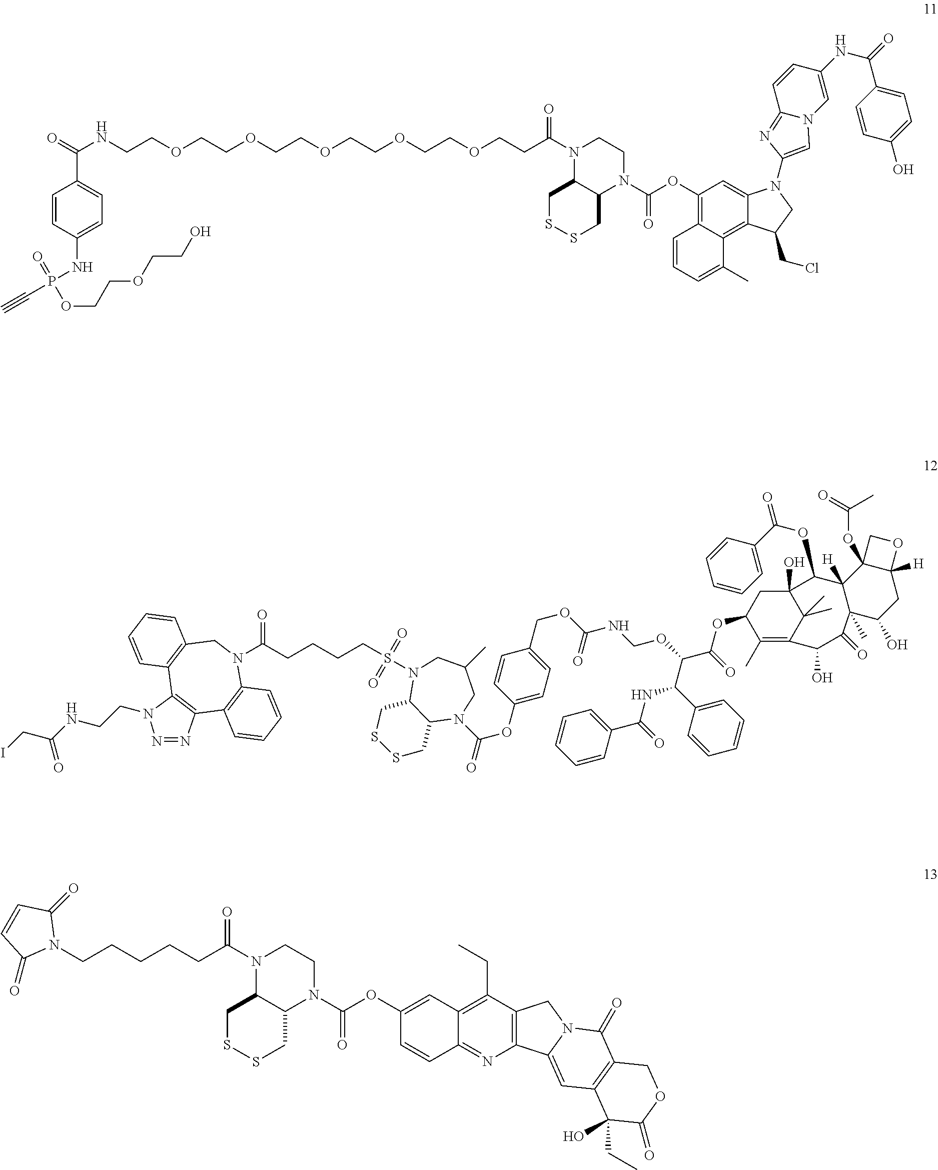

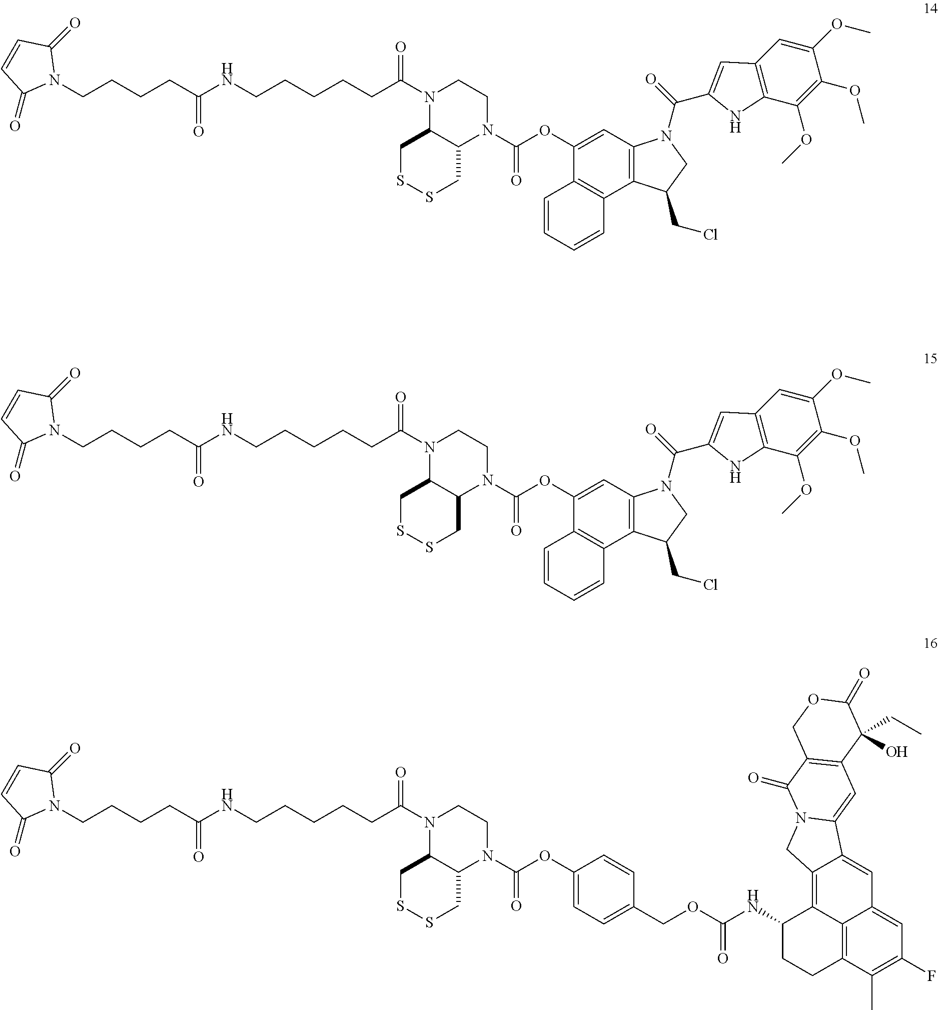

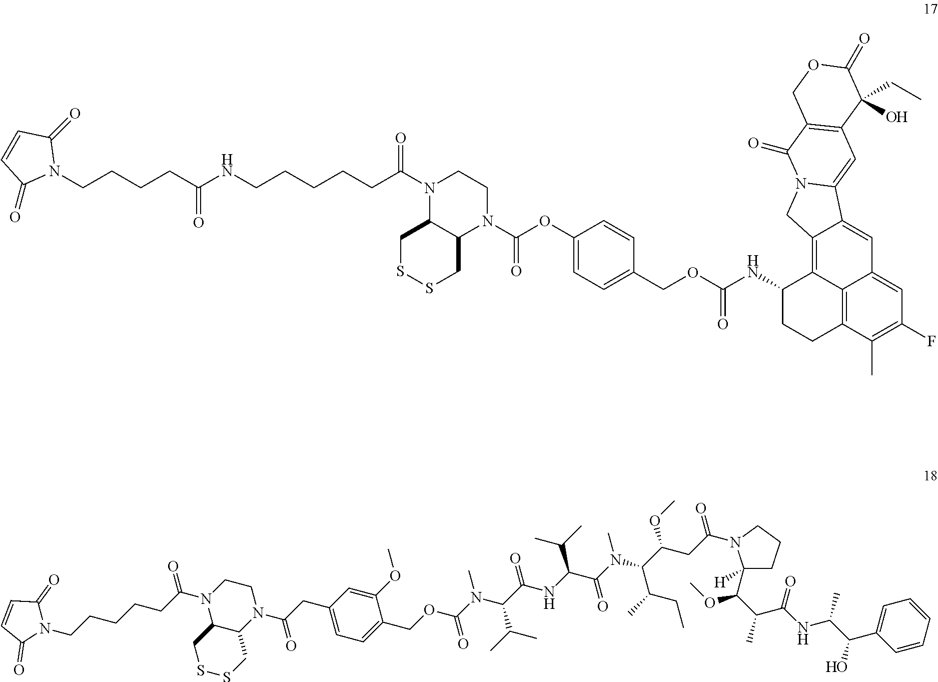

Illustrative embodiments of the compound having the formula (III) include

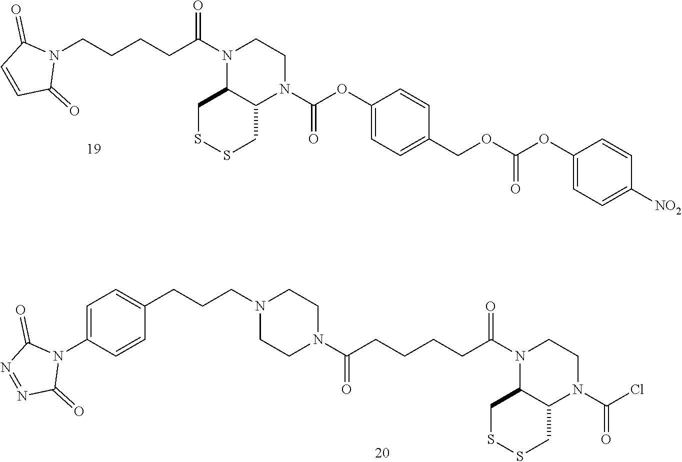

Illustrative embodiments of the compound having the formula (IV) include

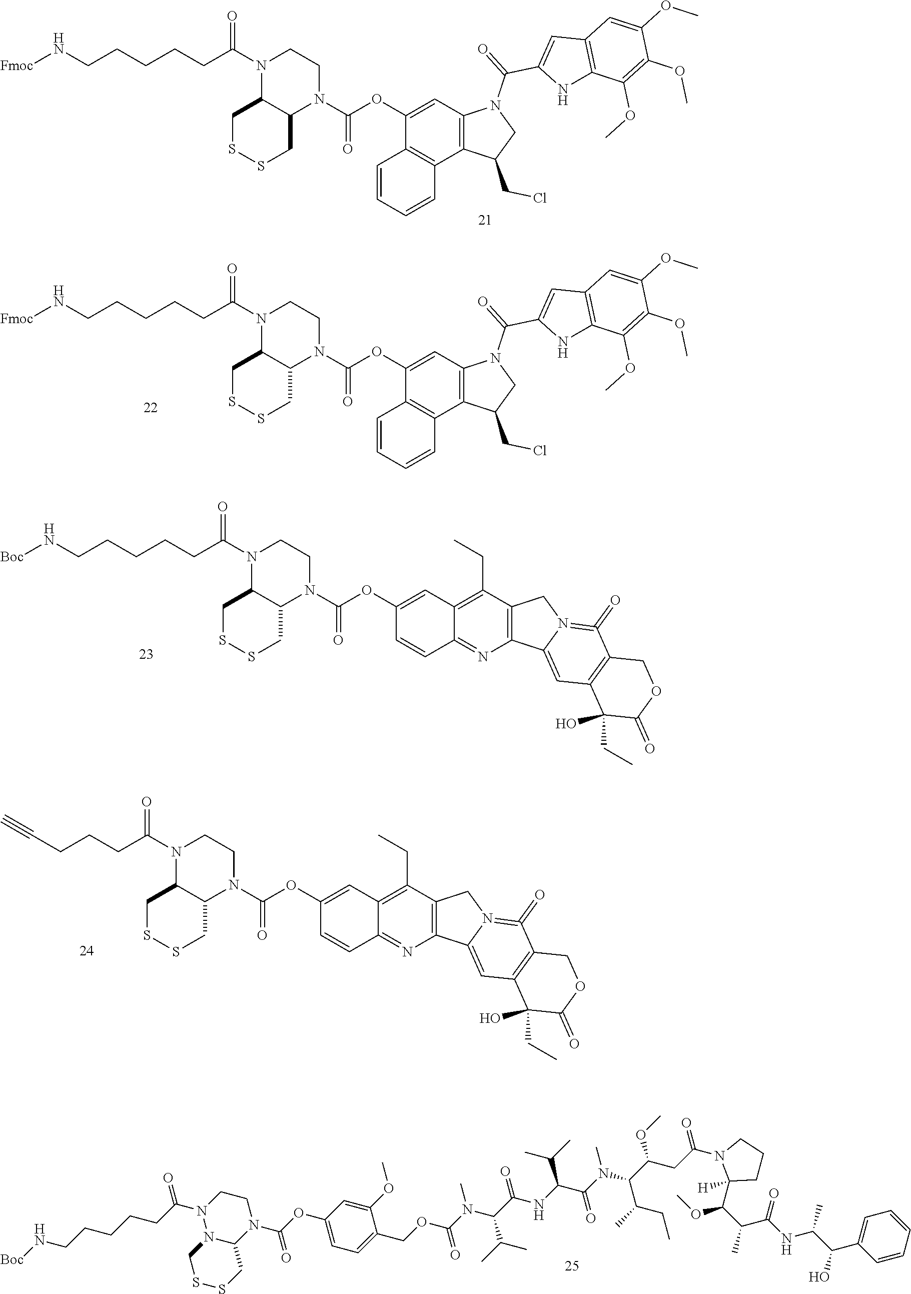

Illustrative embodiments of the compound having the formula (V) include

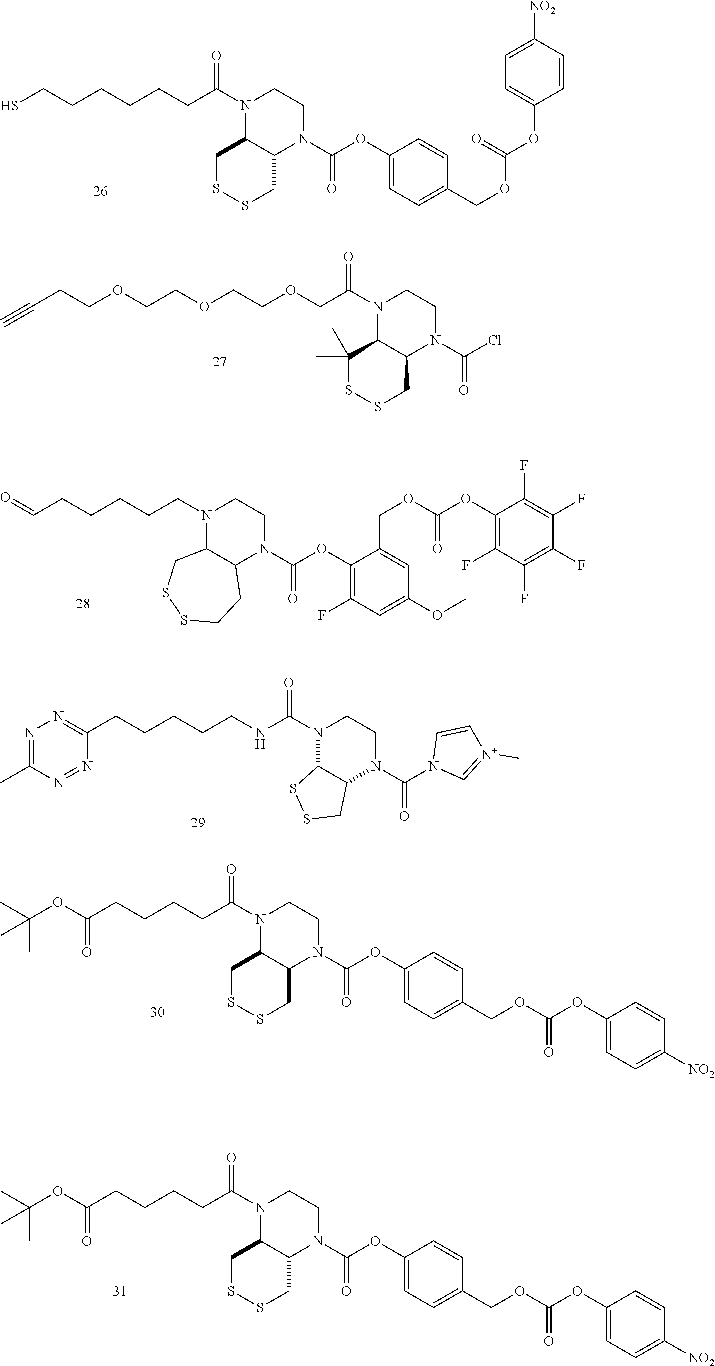

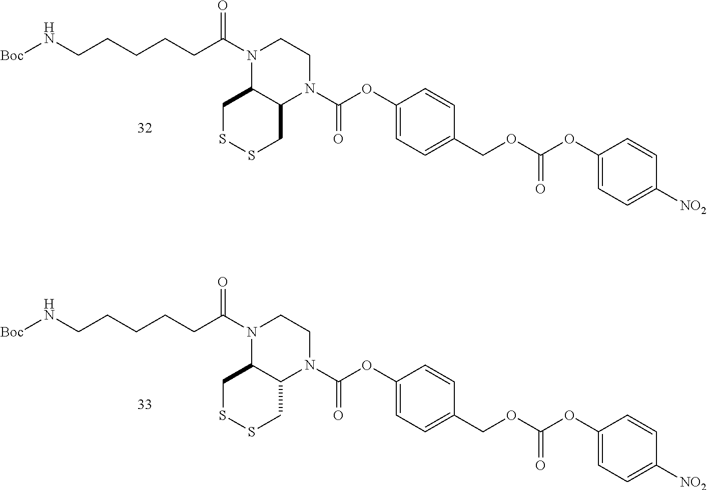

### Pharmaceutical Compositions

The compounds of formula (I) can be administered to a patient in the form of a pharmaceutical composition which can optionally comprise one or more pharmaceutical carrier(s) or excipients.

The compounds of formula (I) can be administered by various well known routes, including oral, rectal and parenteral administration, e.g. intravenous, intramuscular, intradermal, subcutaneous, topical, and similar administration routes. Parenteral, oral and topical administration are preferred, particularly preferred is intravenous administration.

Particular preferred pharmaceutical forms for the administration of a compound of formula (I) are forms suitable for injectable use and include sterile aqueous solutions or dispersions and sterile powders for the extemporaneous preparation of sterile injectable solutions or dispersion. In all cases the final solution or dispersion form must be sterile and fluid. Typically, such a solution or dispersion will include a solvent or dispersion medium, containing, for example, water buffered aqueous solutions, e.g. biocompatible buffers, ethanol, polyol, such as glycerol, propylene glycol, polyethylene glycol, suitable mixtures thereof, surfactants or vegetable oils.

A compound of the invention can also be formulated into liposomes, in particular for parenteral administration. Liposomes provide the advantage of increased half life in the circulation, if compared to the free drug and a prolonged more even release of the enclosed drug.

Sterilization of infusion or injection solutions can be accomplished by any number of art recognized techniques including but not limited to addition of preservatives like anti bacterial or anti-fungal agents, e.g. parabene, chlorobutanol, phenol, sorbic acid or thimersal. Further, isotonic agents, such as sugars or salts, in particular sodium chloride may be incorporated in infusion or injection solutions.

Production of sterile injectable solutions containing one or several of the compounds of the invention is accomplished by incorporating the respective compound in the required amount in the appropriate solvent with various ingredients enumerated above as required followed by sterilization. To obtain a sterile powder the above solutions are vacuum dried or freeze dried as necessary. Preferred diluents of the present invention are water, physiologically acceptable buffers, physiologically acceptable buffer salt solutions or salt solutions. Preferred carriers are cocoa butter and vitebesole. Excipients which can be used with the various pharmaceutical forms of a compound of the invention can be chosen from the following non limiting list:
a) binders such as lactose, mannitol, crystalline sorbitol, dibasic phosphates, calcium phosphates, sugars, microcrystalline cellulose, carboxymethyl cellulose, hydroxyethyl cellulose, polyvinyl pyrrolidone and the like;
b) lubricants such as magnesium stearate, talc, calcium stearate, zinc stearate, stearic acid, hydrogenated vegetable oil, leucine, glycerids and sodium stearyl fumarates,
c) disintegrants such as starches, croscaramellose, sodium methyl cellulose, agar, bentonite, alginic acid, carboxymethyl cellulose, polyvinyl pyrrolidone and the like.

In one embodiment the formulation is for oral administration and the formulation comprises one or more or all of the following ingredients: pregelatinized starch, talc, povidone K 30, croscarmellose sodium, sodium stearyl fumarate, gelatin, titanium dioxide, sorbitol, monosodium citrate, xanthan gum, titanium dioxide, flavoring, sodium benzoate and saccharin sodium.

Other suitable excipients can be found in the Handbook of Pharmaceutical Excipients, published by the American Pharmaceutical Association, which is herein incorporated by reference.

The pharmaceutical compositions comprising a compound of the invention can be produced in a manner known per se to the skilled person as described, for example, in Remington's Pharmaceutical Sciences, 15th Ed., Mack Publishing Co., New Jersey (1991).

It is to be understood that depending on the severity of the disorder and the particular type which is treatable with one of the compounds of the invention, as well as on the respective patient to be treated, e.g. the general health status of the patient, etc., different doses of the respective compound are required to elicit a therapeutic or prophylactic effect.

The cargo is, as defined above, any therapeutic agent. Depending on the therapeutic agent, the mode of administration, amongst other factors, the appropriate dose spans a wide range. The determination of the appropriate dose lies within the discretion of the attending physician.

### Utility, Diseases

The compounds having the formula (I) are particularly useful as therapeutics.

The compounds having the formula (I) can be used in the treatment, amelioration or prevention of a disorder selected from a neoplastic disorder; atherosclerosis; an autoimmune disorder; an inflammatory disease; ischaemia; reperfusion injury; and a chronic inflammatory autoimmune disease.

Preferably, the disorder is a neoplastic disorder, in particular cancer. The cancer is not particularly limited, preferably the cancer is selected from acoustic neuroma, adenocarcinoma, angiosarcoma, basal cell carcinoma, bile duct carcinoma, bladder carcinoma, breast cancer, bronchogenic carcinoma, cervical cancer, chondrosarcoma, chordoma, choriocarcinoma, craniopharyngioma, cystadenocarcinoma, embryonal carcinoma, endotheliosarcoma, ependymoma, epithelial carcinoma, Ewing's tumor, fibrosarcoma, hemangioblastoma, leiomyosarcoma, liposarcoma, Merkel cell carcinoma, melanoma, mesothelioma, myelodysplastic syndrome, myxosarcoma, oligodendroglioma, osteogenic sarcoma, ovarian cancer, pancreatic cancer, papillary adenocarcinomas, papillary carcinoma, pinealoma, prostate cancer, renal cell carcinoma, retinoblastoma, rhabdomyosarcoma, sebaceous gland carcinoma, seminoma, squamous cell carcinoma, sweat gland carcinoma, synovioma, testicular tumor, Wilms' tumor, adrenocortical carcinoma, urothelial carcinoma, gallbladder cancer, parathyroid cancer, Kaposi sarcoma, colon carcinoma, gastrointestinal stromal tumor, anal cancer, rectal cancer, small intestine cancer, brain tumor, glioma, glioblastoma, astrocytoma, neuroblastoma, medullary carcinoma, medulloblastoma, meningioma, leukemias including acute myeloid leukemia, multiple myeloma, acute lymphoblastic leukemia, liver cancer including hepatoma and hepatocellular carcinoma, lung carcinoma, non-small-cell lung cancer, small cell lung carcinoma, lymphangioendotheliosarcoma, lymphangiosarcoma, primary CNS lymphoma, non-Hodgkin lymphoma, and classical Hodgkin's lymphoma. The compounds of the present invention are particularly useful against colon cancer, rectal cancer, small intestine cancer, brain tumor, leukemia, liver cancer, lung cancer, lymphoma, basal cell carcinoma, breast cancer, cervical cancer, melanoma, ovarian cancer, pancreatic cancer, and squamous cell carcinoma.

The mechanism by which reductive cleavage of the disulfide or selenenylsulfide can lead to release of the therapeutically active drug from a compound of formula (I) is shown illustratively in the following scheme. This example depicts the release of H-L-B following reduction and cyclisation of a compound of formula (I). The reducing agent should, in cells, preferably be a reducing enzyme or protein with a dithiol or selenolthiol active site, for example thioredoxin, thioredoxin reductase, or glutathione reductase. H-L-B then self-immolates to release H—B, as is well-known to those skilled in the art (see e.g. Carl et al. J. Med. Chem. 1981, 24, 479).

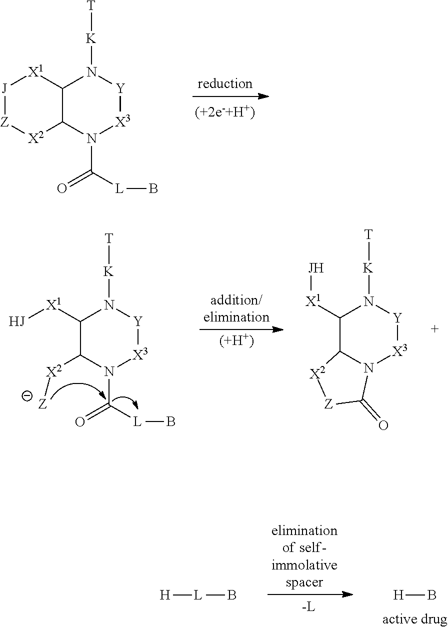

The molecules of formula (II), (III), (IV) and (V) are particularly useful for the preparation of compounds of formula (I). For example, as illustrated in the scheme below, Y5 is a compound of formula (I) that can be assembled from Y1 which is a compound of formula (V); or Y2 which is a compound of formula (III); or Y3 which is a compound of formula (IV); or Y4 which is a compound of formula (II), by standard reaction types that will be known to those skilled in the art. Brentuximab is shown as a targeting unit for illustration purposes. However, the reaction scheme can be easily adapted to other targeting units and Drug-To-Antibody ratios as appropriate by a skilled person.

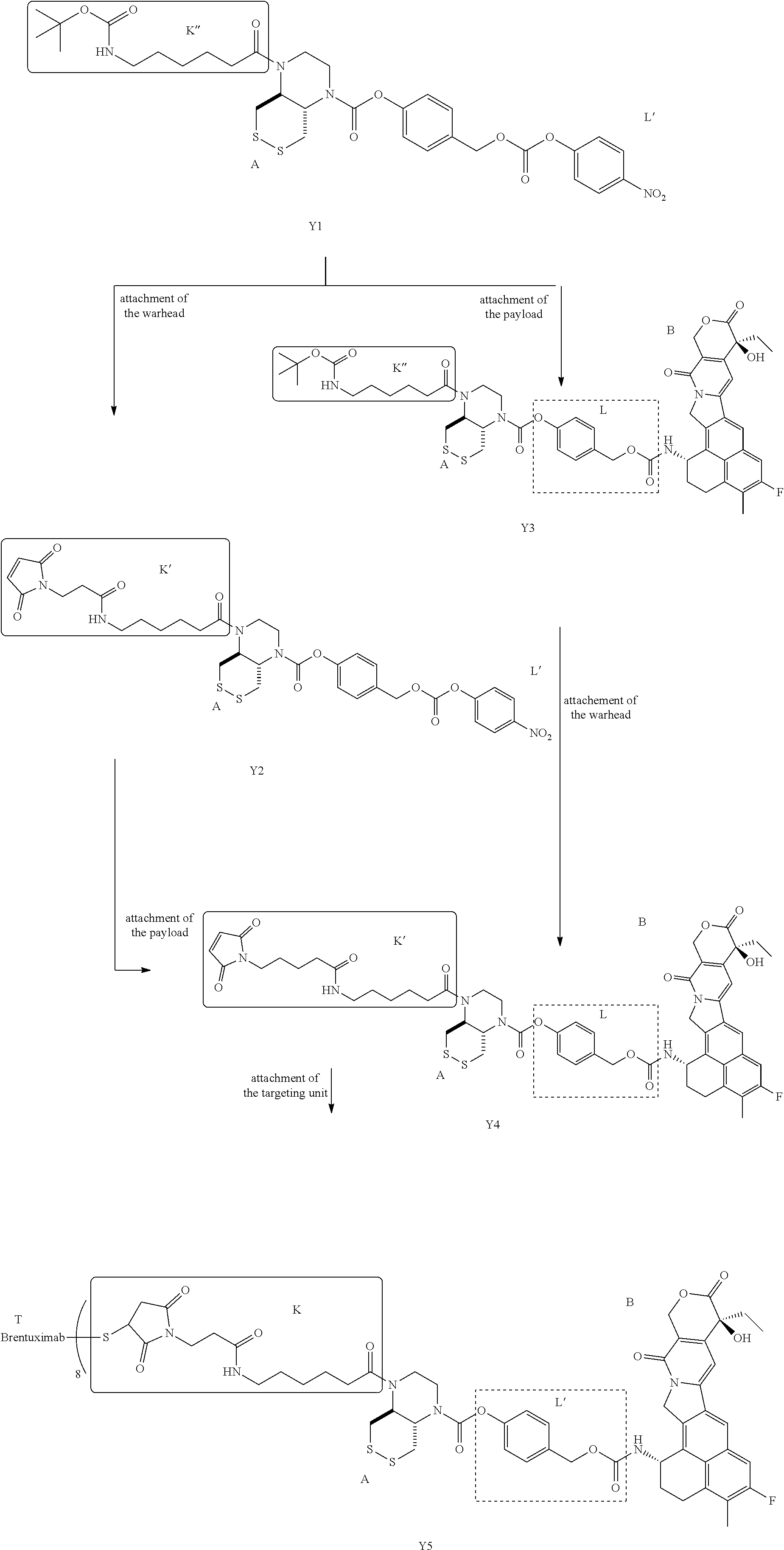

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

A key aspect of this invention is that the novel class of fused cyclic dichalcogenide-based cleavable units (A) dictate the therapeutic performance of the therapeutic compound of formula (I) (T-K-A-L-B). The cleavable units' key properties, i.e. (bio-) chemical robustness in plasma or outside target cells, yet exceptionally fast intracellular reduction-to-release kinetics allowing cleavage after entry to the target cells, render them widely applicable as a molecular motif that is key to unlocking therapeutic performance across diverse constructs containing different targeting units (T) and drugs (B): since this low off-target exposure but efficient on-target release will maximise the on-target-to-off-target drug delivery ratio, and thereby protect healthy tissues during an effective therapy.

### Examples

The invention will be more fully understood by reference to the following examples. They should not, however, be construed as limiting the scope of the invention.

### 1. Abbreviations

aq.: aqueous (solution)
Boc: tert-butoxycarbonyl
calc: calculated
COMU: 1-[1-(cyano-2-ethoxy-2-oxoethylideneaminooxy)-dimethylamino-morpholino]-uronium hexafluorophosphate
DCM: dichloromethane
DMF: dimethylformamide
DMSO: dimethylsulfoxide
(D)PBS: (Dulbecco's) phosphate buffered saline
EI: electron ionization
ESI: electron spray ionization
FA: formic acid
FCC: flash column chromatography
HATU: hexafluorophosphate azabenzotriazole tetramethyl uronium
HBTU: hexafluorophosphate benzotriazole tetramethyl uronium
(HP) LC: (high performance) liquid chromatography
HRMS: high-resolution mass spectrometry
DIPEA: diisopropylethylamine
J: coupling constant
LC-MS: liquid chromatography mass spectrometry
LRMS: low-resolution mass spectrometry
NMR: nuclear magnetic resonance
(δ) ppm: chemical shift (parts per million)
R_{f}: retention factor
r.t.: room temperature
sat.: saturated (solution)
TEA: triethylamine
THF: tetrahydrofuran
TLC: thin layer chromatography

### 2. General Synthesis Conditions

Unless stated otherwise, (1) all reactions and characterisations were performed with solvents and reagents, used as obtained, under closed air atmosphere without special precautions; (2) DCM, Et₂O, THF and DMF used for synthesis were distilled, then dried on activated molecular sieves; (3) isohexane and EtOAc used for chromatography was distilled from commercial crude iso-hexane fraction; (4) “column” and “chromatography” refer to FCC, performed on Merck silica gel Si-60 (40-63 μm); (5) procedures and yields are unoptimised; (6) yields refer to isolated chromatographically and spectroscopically pure materials, corrected for residual solvent content.

NMR: Standard NMR characterisation was carried out by ¹H- and ¹³C-NMR spectra. Avance III Bruker BioSpin 400 MHZ, 500 MHz and 600 MHz spectrometers were used (¹H: 400 MHZ, 500 MHz or 600 MHz, ¹³C: 101 MHZ, 126 MHz and 151 MHz respectively) typically at 298 K, although with some carbamates spectroscopy was simplified by measuring at 373 K to induce a dynamic equilibrium between their rotameric species. Chemical shifts (δ) are reported in ppm calibrated to residual non-perdeuterated solvent as an internal reference. Peak descriptions singlet(s), doublet (d), triplet (t), quartet (q), pentuplet (p), and multiplet (m) are used. Coupling constants J are given in Hz.

Mass spectra: Unit mass measurements were performed on an AGILENT 1200 SL coupled LC-MS system with ESI mode ionisation, with binary eluent mixtures of H₂O: MeCN, with the water containing formic acid. HRMS was carried out by the Zentrale Analytik of the Ludwig-Maximilian-University (LMU), Munich (electron impact (EI) at 70 eV with a Thermo Finnigan MAT 95 or a Jeol GCmate II spectrometer; electrospray ionization (ESI) with a Thermo Finnigan LTQ FT Ultra Fourier Transform Ion Cyclotron resonance mass spectrometer) in positive or negative mode as stated.

### 3. Synthesis

### (A) Camptothecin-ADC Precursor—Example of a Compound of the Formula (II)

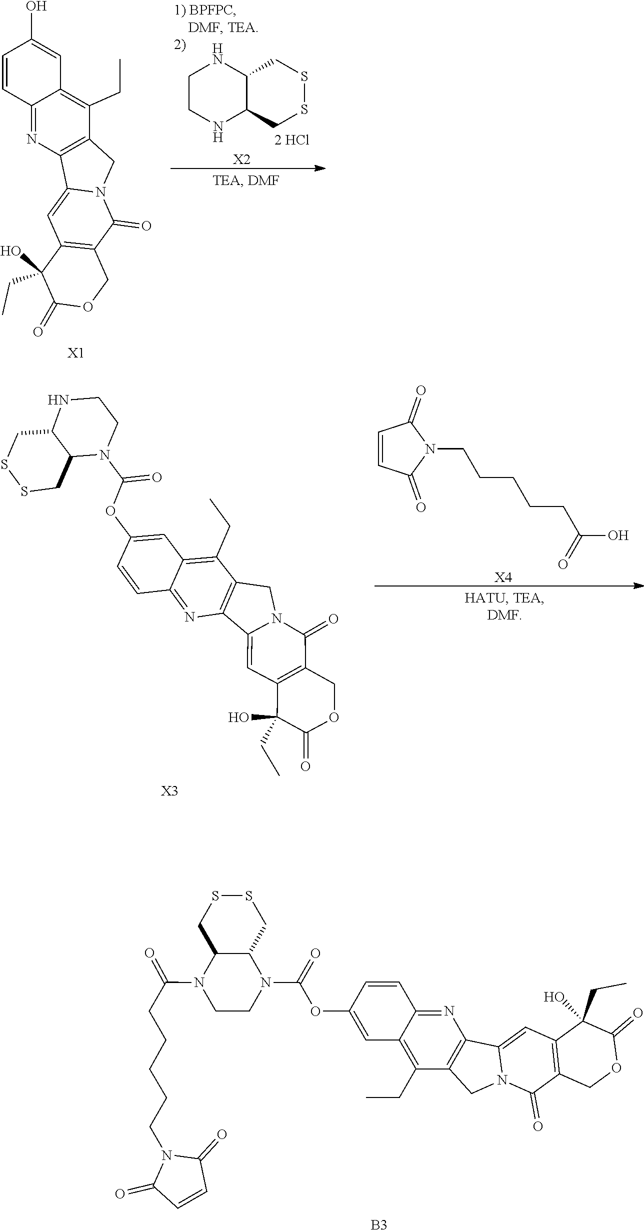

### (S)-4,11-diethyl-4-hydroxy-3,14-dioxo-3,4,12,14-tetrahydro-1H-pyrano[3′,4′: 6,7]indolizino[1,2-b]quinolin-9-yl hexahydro-[1,2]dithiino[4,5-b]pyrazine-1(2H)-carboxylate (trans) (X3)

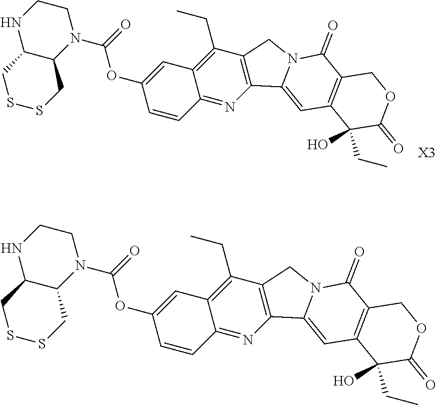

7-ethyl-10-hydroxycamtothecin (X1) (100 mg, 0.26 mmol) was dissolved in anhydrous DMF (5 mL, 0.05 M) and TEA (100 mg, 137 μL, 0.99 mmol) was added, followed by a solution of bis(pentafluorophenyl) carbonate (100 mg, 0.26 mmol) in anhydrous DMF (0.2 M). The resulting clear solution was stirred at r.t. for 30 min. Separately, X2 (·2 HCl·2.5 NaCl) (100 mg, 71 w %, 0.28 mmol) was suspended in anhydrous DMF (6 mL, 0.05 M) and TEA (100 mg, 137 μL, 0.99 mmol) was added. The clear solution from step 1 was added dropwise to the suspension of X2 at r.t. and the resulting mixture was further stirred at r.t. for 2 h. Purification was achieved by preparative HPLC (H₂O/MeCN, 0.1% FA) to yield the title compound X3 as a colourless solid (41 mg, 0.07 mmol, 27%).

TLC R_{f}=0.21 (DCM: MeOH, 6:1). HRMS (ESI): C₂₉H₃₁N₄O₆S₂⁺: [M+NH₄]⁺ calc. 595.16795, found 595.16719. ¹H-NMR (500 MHZ, CDCl₃) δ (ppm)=8.17 (d, J=9.1 Hz, 1H), 8.00 (d, J=2.3 Hz, 1H), 7.67 (dd, J=9.1, 2.3 Hz, 1H), 7.32 (s, 1H), 6.53 (s, 1H), 5.43 (s, 2H), 5.32 (s, 2H), 3.91-3.79 (m, 2H), 3.64-3.53 (m, 1H), 3.28 (d, J=10.1 Hz, 2H), 3.22-3.07 (m, 4H), 3.07-2.97 (m, 2H), 2.97-2.80 (m, 2H), 1.86 (dh, J=21.4, 7.3 Hz, 2H), 1.30 (t, J=7.6 Hz, 3H), 0.88 (t, J=7.3 Hz, 3H). ¹³C-NMR (126 MHz, CDCl₃): δ (ppm)=172.5, 156.8, 153.4, 151.7, 150.0, 149.8, 146.3, 145.9, 145.2, 131.0, 128.5, 127.0, 126.0, 119.6, 115.0, 96.6, 72.4, 65.3, 63.7, 56.2, 55.8, 49.6, 43.9, 36.8, 30.3, 22.2, 13.5, 7.8.

### (S)-4,11-diethyl-4-hydroxy-3,14-dioxo-3,4,12,14-tetrahydro-1H-pyrano[3′,4′:6,7]indolizino[1,2-b]quinolin-9-yl 4-(6-(2,5-dioxo-2,5-dihydro-1H-pyrrol-1-yl) hexanoyl)hexahydro-[1,2]dithiino[4,5-b]pyrazine-1(2H)-carboxylate (trans) (B3)

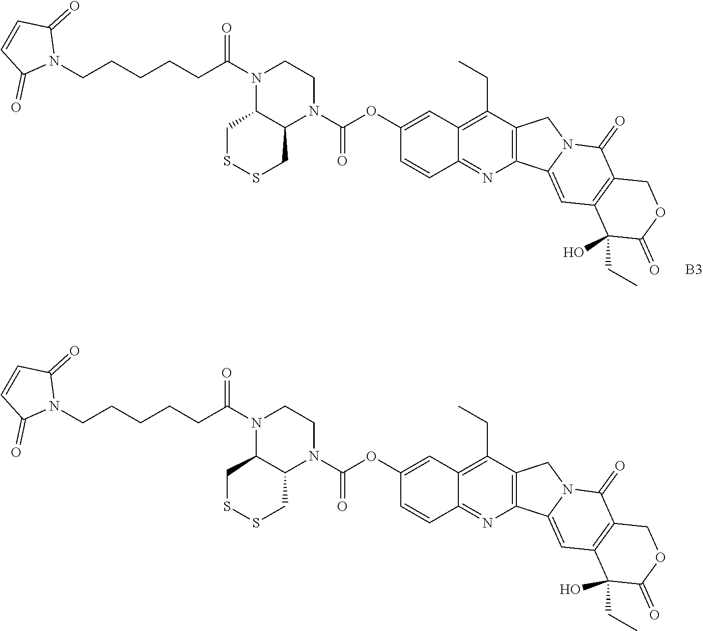

X4 (5.3 mg, 0.025 mmol) was dissolved in anhydrous DMF (0.5 mL, 0.05 M) and TEA (7 μL, 0.050 mmol) and HATU (9.6 mg, 0.025 mmol) were added at r.t. A solution of X3 (10.0 mg, 0.017 mmol) in DMF (0.2 mL, 0.02 M) was added to the solution and the resulting mixture was stirred at r.t. for 2 h. Purification was achieved by preparative HPLC (H₂O/MeCN, 0.1% FA) to yield the title compound B3 as a colourless solid (11.0 mg, 0.014 mmol, 82%).

TLC R_{f}=0.25 (DCM: MeOH, 12:1). HRMS (ESI): C₃₉H₄₂N₅O₉S₂⁺: [M+H]⁺ calc. 788.24185, found 788.24042. ¹H-NMR (500 MHZ, DMSO-d₆) δ (ppm)=8.18 (d, J=9.1 Hz, 1H), 7.93 (s, 1H), 7.58 (d, J=8.7 Hz, 1H), 7.32 (s, 1H), 6.98 (s, 2H), 6.52 (s, 1H), 5.44 (s, 2H), 5.33 (s, 2H), 4.31 (t, J=9.7 Hz, 1H), 4.04 (s, 2H), 3.80 (s, 1H), 3.71 (s, 3H), 3.54-3.42 (m, 2H), 3.39-3.34 (m, 2H), 3.24-3.12 (m, 2H), 3.07 (d, J=11.1 Hz, 1H), 2.44-2.31 (m, 2H), 1.87 (dp, J=21.4, 7.2 Hz, 2H), 1.51 (dt, J=21.6, 7.0 Hz, 4H), 1.29 (t, J=7.6 Hz, 3H), 1.26-1.22 (m, 2H), 0.88 (t, J=7.3 Hz, 3H). ¹³C-NMR (126 MHz, DMSO-d₆): δ (ppm)=172.5, 171.1, 156.8, 151.9, 150.0, 149.4, 146.4, 145.9, 145.2, 134.4, 131.1, 128.6, 127.0, 125.7, 119.0, 114.9, 96.6, 72.4, 65.3, 58.5, 49.6, 36.9, 32.8, 30.3, 27.8, 25.8, 24.3, 22.2, 13.8, 7.8.

### (B) Duocarmycin-ADC Precursor—Examples of Compounds of Formula (IV)

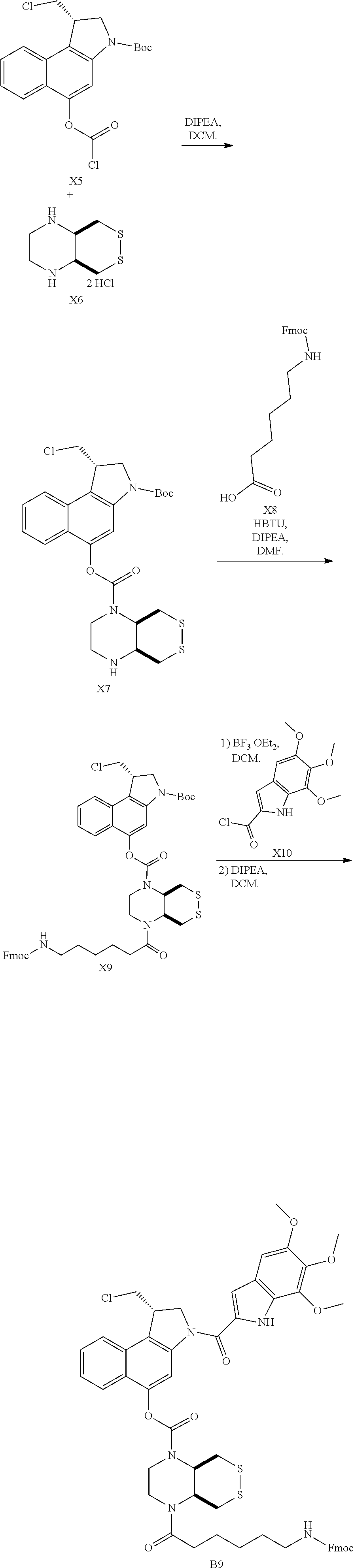

### (S)-3-(tert-butoxycarbonyl)-1-(chloromethyl)-2,3-dihydro-1H-benzo[e]indol-5-yl 4-(6-((((9H-fluoren-9-yl)methoxy)carbonyl)amino)hexanoyl)hexahydro-[1,2]dithiino[4,5-b]pyrazine-1(2H)-carboxylate (cis) (X9)

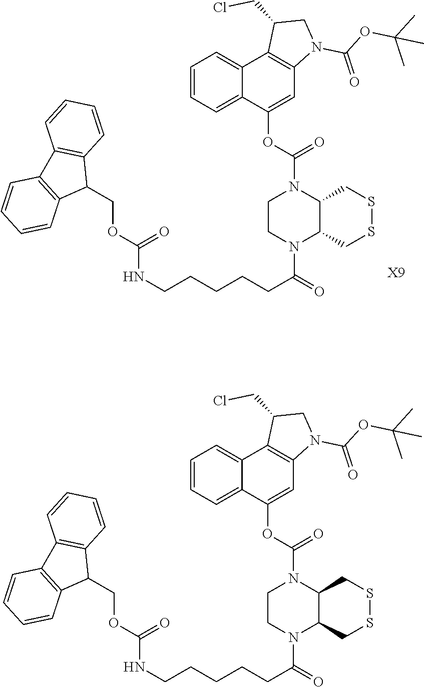 [SMILES: CC(C)(C)OC(=O)N1C[C@@H](CCl)c2c1cc(OC(=O)N1CCN(C(=O)CCCCCNC(=O)OCC3c4ccccc4-c4ccccc43)[C@H]3CSSC[C@H]31)c1ccccc21 ; "X9" ; CC(C)(C)OC(=O)N1C[C@@H](CCl)c2c1cc(OC(=O)N1CCN(C(=O)CCCCCNC(=O)OCC3c4ccccc4-c4ccccc43)[C@@H]3CSSC[C@@H]31)c1ccccc21]

Step 1: X6 (·2 HCl·2.5 NaCl) (139 mg, 64 w %, 0.351 mmol, 1.2 eq.) was suspended in anhydrous DCM (20 mL, 0.015 M), DIPEA (149 μL, 0.878 mmol, 3.0 eq) was added at r.t. and the resulting mixture was stirred for 30 min. X5 was prepared according to reported methods (Felber et al. ACS Cent. Sci. 2023, 9, 4, 763). A solution of X5 (2.9 mL, 0.1 M in anhydrous DCM, 0.293 mmol, 1.0 eq.) was added dropwise at 0° C., and the resulting mixture was stirred at 0° C. for 30 min, was then allowed to warm to r.t. and was further stirred for 1 h, before being concentrated under reduced pressure. Purification of the intermediate was achieved by FCC (EtOAc) to elute X7 (153 mg) with satisfying purity. TLC R_{f}=0.35 (EtOAc).

Step 2: X8 (61.7 mg, 0.175 mmol, 1.2 eq.) was dissolved in anhydrous DMF (3 mL, 0.05 M) and HBTU (66.2 mg, 0.175 mmol, 1.2 eq.) and DIPEA (75 μL, 0.582 mmol, 4.0 eq) were added, and the resulting mixture was stirred at r.t. for 10 min. A solution of X7 (78 mg, 50 w % of the material obtained in step 1) in 0.7 mL of anhydrous DMF was added dropwise, and the resulting mixture was stirred for 15 h before being concentrated under reduced pressure. Purification by FCC (isohexane/EtOAc) gave title compound X9 as a colorless solid (29.3 mg, 0.034 mmol, 23% over 4 steps).

TLC R_{f}=0.87 (EtOAc); 0.23 (isohexane:EtOAc, 1:1). HRMS (ESI): C₄₆H₅₅CIN₅O₇S₂⁺: [M+NH₄]⁺ calc. 888.32259, found 888.32234. ¹H-NMR (500 MHZ, CDCl₃) δ (ppm)=8.07 (s, 1H), 7.76 (d, J=7.5 Hz, 3H), 7.71 (d, J=8.4 Hz, 1H), 7.59 (d, J=7.4 Hz, 2H), 7.54-7.48 (m, 1H), 7.39 (q, J=6.5, 5.7 Hz, 3H), 7.31 (t, J=7.4 Hz, 2H), 4.89 (s, 1H), 4.83-4.57 (m, 1H), 4.45 (s, 1H), 4.39 (d, J=6.9 Hz, 2H), 4.35-4.26 (m, 1H), 4.21 (t, J=6.8 Hz, 1H), 4.18-4.11 (m, 1H), 4.02 (t, J=8.4 Hz, 1H), 3.93 (d, J=10.0 Hz, 2H), 3.85-3.74 (m, 2H), 3.60-3.50 (m, 1H), 3.50-3.43 (m, 1H), 3.29-3.16 (m, 3H), 2.99 (d, J=12.1 Hz, 1H), 2.39 (s, 2H), 1.80-1.67 (m, 2H), 1.58 (s, 9H), 1.47-1.38 (m, 2H). ¹³C-NMR (126 MHz, CDCl₃): δ (ppm)=172.7, 156.6, 153.9, 152.5, 148.0, 144.1, 141.5, 130.4, 127.8, 127.8, 127.2, 125.2, 124.7, 122.6, 122.5, 120.1, 109.4, 66.7, 53.1, 51.8, 47.4, 46.4, 42.9, 42.3, 40.9, 36.3, 33.5, 29.9, 29.8, 28.6, 28.3, 26.5, 24.5.

### (S)-1-(chloromethyl)-3-(5,6,7-trimethoxy-1H-indole-2-carbonyl)-2,3-dihydro-1H-benzo[e]indol-5-yl 4-(6-((((9H-fluoren-9-yl)methoxy)carbonyl)amino)hexanoyl)hexahydro-[1,2]dithiino[4,5-b]pyrazine-1(2H)-carboxylate (cis) (B9)

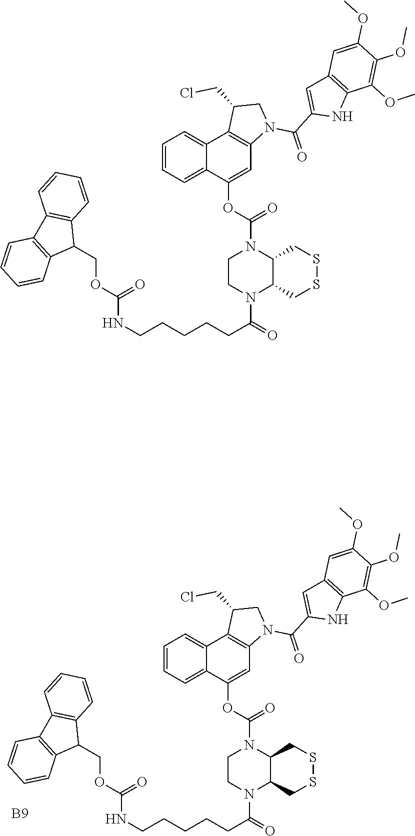 [SMILES: COc1cc2cc(C(=O)N3C[C@@H](CCl)c4c3cc(OC(=O)N3CCN(C(=O)CCCCCNC(=O)OCC5c6ccccc6-c6ccccc65)[C@H]5CSSC[C@H]53)c3ccccc43)[nH]c2c(OC)c1OC ; "B9" ; COc1cc2cc(C(=O)N3C[C@@H](CCl)c4c3cc(OC(=O)N3CCN(C(=O)CCCCCNC(=O)OCC5c6ccccc6-c6ccccc65)[C@@H]5CSSC[C@@H]53)c3ccccc43)[nH]c2c(OC)c1OC]

Step 1: X8 (20.0 mg, 0.023 mmol) was dissolved in anhydrous DCM (0.02 M) and BF₃·OEt₂ (15 μL, 0.12 mmol, 5.0 eq.) was added dropwise at r.t. and the mixture was stirred at r.t. for 30 min, before being concentrated under reduced pressure to yield the free amine intermediate as a brown solid, that was used without further purification.

Step 2: The material obtained in step 1 was suspended in anhydrous DCM (0.05 M), DIPEA (12 μL, 0.07 mmol, 3.0 eq.) was added, and a clear solution was observed. The mixture was then added dropwise at r.t. to a solution of X10 (9.3 mg, 0.034 mmol, 1.5 eq.) (pre-activated with (COCl)₂/DMF), the resulting mixture was stirred at r.t. for 1 h. Complete turnover as indicated by TLC. The mixture was poured into 5 mL sat. aq. NaCl and 5 mL water was added. The mixture was extracted with DCM (4×10 mL). The combined organic layers were dried over Na₂SO₄ and concentrated under reduced pressure. Purification by FCC (isohexane: EtOAc; 1:1 to 0:1) gave B9 as a colorless solid (17.7 mg, 0.018 mmol, 78%).

TLC R_{f}=0.61 (EtOAc). HRMS (ESI): C₅₃H₅₃CIN₅O₉S₂: [M−H]⁻ calc. 1002.29787, found 1002.29657. ¹H-NMR (400 MHZ, CDCl₃) δ (ppm)=9.37 (s, 1H), 8.46-8.34 (m, 1H), 7.82 (dd, J=13.4, 5.7 Hz, 1H), 7.76 (d, J=7.5 Hz, 5H), 7.59 (d, J=7.4 Hz, 4H), 7.56-7.50 (m, 1H), 7.50-7.44 (m, 1H), 7.39 (t, J=7.5 Hz, 4H), 7.30 (t, J=7.3 Hz, 4H), 7.03-6.99 (m, 1H), 6.90-6.87 (m, 1H), 4.99-4.87 (m, 1H), 4.82 (d, J=10.7 Hz, 1H), 4.76-4.64 (m, 1H), 4.54-4.45 (m, 1H), 4.39 (d, J=6.6 Hz, 4H), 4.36-4.29 (m, 1H), 4.26-4.14 (m, 2H), 4.08 (s, 3H), 4.02-3.96 (m, 2H), 3.95 (s, 3H), 3.92 (s, 3H), 3.85-3.72 (m, 2H), 3.64-3.43 (m, 2H), 3.30-3.11 (m, 5H), 3.00 (d, J=11.9 Hz, 1H), 2.47-2.31 (m, 3H), 1.80-1.67 (m, 4H), 1.65-1.52 (m, 4H), 1.52-1.37 (m, 4H). ¹³C-NMR (101 MHZ, CDCl₃): δ (ppm)=173.2, 156.6, 153.9, 150.4, 147.8, 144.1, 141.4, 141.0, 138.8, 130.0, 128.1, 127.8, 127.2, 125.6, 125.2, 123.7, 122.9, 122.6, 120.1, 111.4, 106.8, 97.7, 66.7, 61.6, 61.3, 56.4, 55.0, 51.9, 47.4, 46.0, 43.5, 40.9, 36.3, 33.5, 31.3, 29.8, 26.5, 24.5, 22.5, 14.3.

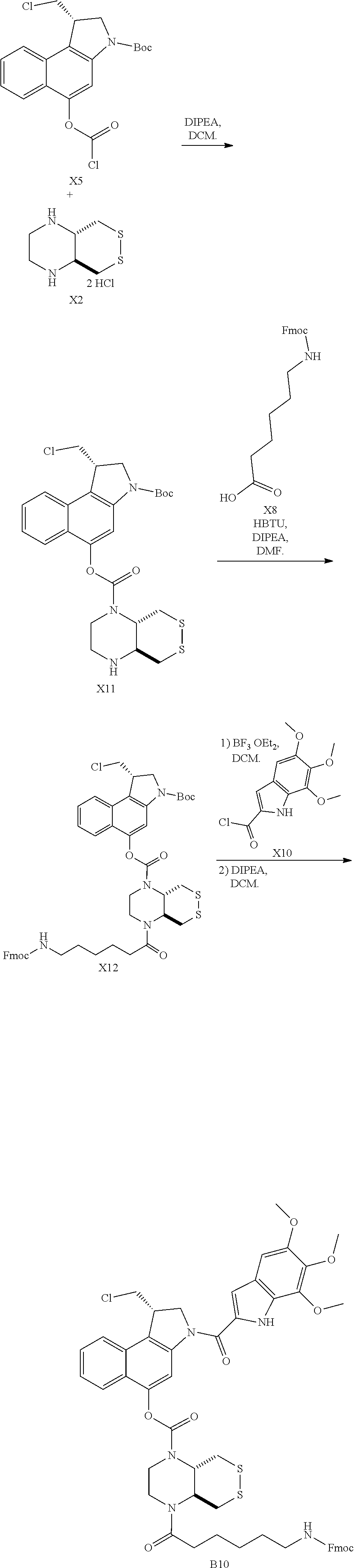

### (S)-3-(tert-butoxycarbonyl)-1-(chloromethyl)-2,3-dihydro-1H-benzo[e]indol-5-yl 4-(6-((((9H-fluoren-9-yl)methoxy)carbonyl)amino)hexanoyl)hexahydro-[1,2]dithiino[4,5-b]pyrazine-1(2H)-carboxylate (trans) (X12)

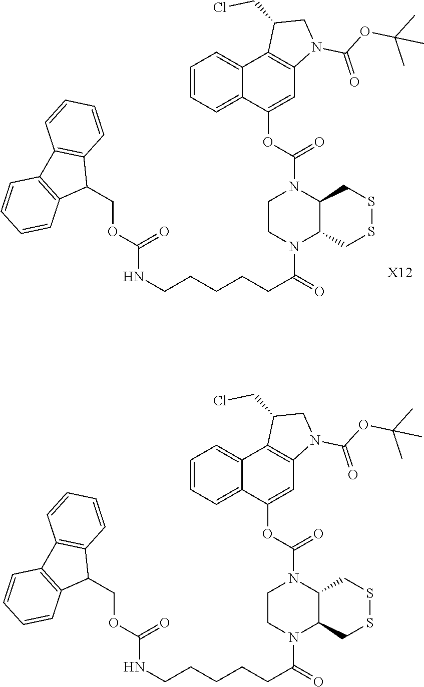 [SMILES: CC(C)(C)OC(=O)N1C[C@@H](CCl)c2c1cc(OC(=O)N1CCN(C(=O)CCCCCNC(=O)OCC3c4ccccc4-c4ccccc43)[C@H]3CSSC[C@@H]31)c1ccccc21 ; "X12" ; CC(C)(C)OC(=O)N1C[C@@H](CCl)c2c1cc(OC(=O)N1CCN(C(=O)CCCCCNC(=O)OCC3c4ccccc4-c4ccccc43)[C@@H]3CSSC[C@H]31)c1ccccc21]

Step 1: X2 (0.2 HCl-2.5 NaCl) (139 mg, 64 w %, 0.351 mmol, 1.2 eq.) was suspended in anhydrous DCM (20 mL, 0.015 M), DIPEA (149 μL, 0.878 mmol, 3.0 eq) was added at r.t. and the resulting mixture was stirred for 30 min. X5 was prepared according to reported methods (Felber et al. ACS Cent. Sci. 2023, 9, 4, 763). A solution of X5 (2.9 mL, 0.1 M in anhydrous DCM, 0.293 mmol, 1.0 eq.) was added dropwise at 0° C., and the resulting mixture was stirred at 0° C. for 30 min, was then allowed to warm to r.t. and was further stirred for 1 h, before being concentrated under reduced pressure. Purification of the intermediate was achieved by FCC (EtOAc) to elute X11 (215 mg) with satisfying purity. TLC R_{f}=0.53 (EtOAc).

Step 2: X8 (61.7 mg, 0.175 mmol, 1.2 eq.) was dissolved in anhydrous DMF (3 mL, 0.05 M) and HBTU (66.2 mg, 0.175 mmol, 1.2 eq.) and DIPEA (75 μL, 0.582 mmol, 4.0 eq) were added, and the resulting mixture was stirred at r.t. for 10 min. A solution of X11 (107 mg, 50 w % of the material obtained in step 1 with estimated content of 73%) in 1.0 mL of anhydrous DMF was added dropwise, and the resulting mixture was stirred for 15 h before being concentrated under reduced pressure. Purification by FCC (isohexane/EtOAc) gave the title compound X12 as a colorless solid (51.5 mg, 0.059 mmol, 41% over 4 steps).

TLC R_{f}=0.83 (EtOAc); 0.16 (isohexane:EtOAc, 1:1). HRMS (ESI): C₄₆H₅₅CIN₅O₇S₂+: [M+NH₄]⁺ calc. 888.32259, found 888.32231. ¹H-NMR (500 MHZ, CDCl₃) δ (ppm)=8.01 (d, J=8.3 Hz, 1H), 7.75 (t, J=7.5 Hz, 3H), 7.71-7.65 (m, 2H), 7.57 (d, J=7.3 Hz, 2H), 7.53-7.47 (m, 1H), 7.38 (q, J=7.3 Hz, 3H), 7.29 (t, J=7.4 Hz, 2H), 4.37 (s, 2H), 4.27 (d, J=39.5 Hz, 2H), 4.17 (d, J=12.3 Hz, 1H), 4.16-4.08 (m, 2H), 4.00 (d, J=9.6 Hz, 1H), 3.90 (d, J=11.2 Hz, 1H), 3.73 (s, 4H), 3.45 (t, J=10.8 Hz, 1H), 3.36 (d, J=12.0 Hz, 1H), 3.26-3.19 (m, 1H), 3.19-3.03 (m, 2H), 2.41 (s, 2H), 1.72-1.66 (m, 2H), 1.57 (s, 9H), 1.53-1.44 (m, 2H). ¹³C-NMR (126 MHZ, CDCl₃): δ (ppm)=156.6, 152.5, 147.9, 144.1, 142.6, 141.4, 130.3, 127.8, 127.2, 127.2, 125.2, 125.0, 124.7, 124.0, 122.5, 120.1, 110.6, 109.4, 81.3, 66.7, 60.5, 59.5, 53.0, 50.2, 47.4, 47.2, 46.3, 44.4, 42.0, 40.8, 34.6, 33.9, 32.1, 30.5, 29.8, 29.7, 29.5, 28.5, 26.4, 24.8, 24.3, 22.8, 21.2.

### (S)-1-(chloromethyl)-3-(5,6,7-trimethoxy-1H-indole-2-carbonyl)-2,3-dihydro-1H-benzo[e]indol-5-yl 4-(6-((((9H-fluoren-9-yl)methoxy)carbonyl)amino)hexanoyl)hexahydro-[1,2]dithiino[4,5-b]pyrazine-1(2H)-carboxylate (trans) (B10)

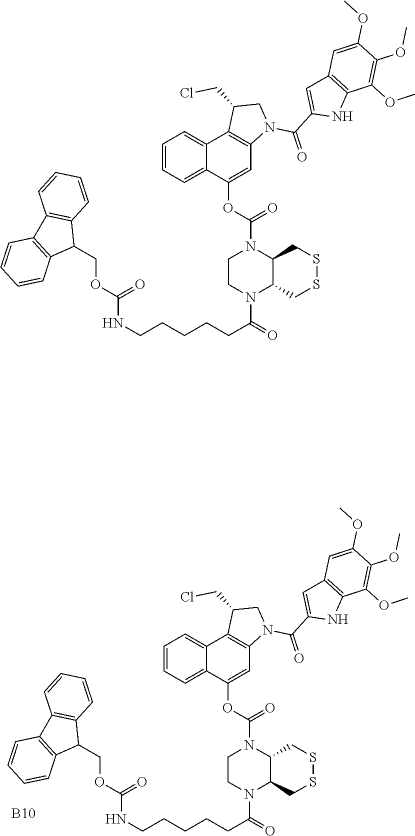 [SMILES: COc1cc2cc(C(=O)N3C[C@@H](CCl)c4c3cc(OC(=O)N3CCN(C(=O)CCCCCNC(=O)OCC5c6ccccc6-c6ccccc65)[C@H]5CSSC[C@@H]53)c3ccccc43)[nH]c2c(OC)c1OC ; "B10" ; COc1cc2cc(C(=O)N3C[C@@H](CCl)c4c3cc(OC(=O)N3CCN(C(=O)CCCCCNC(=O)OCC5c6ccccc6-c6ccccc65)[C@@H]5CSSC[C@H]53)c3ccccc43)[nH]c2c(OC)c1OC]

Step 1: X12 (25.0 mg, 0.029 mmol) was dissolved in anhydrous DCM (0.02 M) and BF₃·OEt₂ (18 μL, 0.14 mmol, 5.0 eq.) was added dropwise at r.t. and the mixture was stirred at r.t. for 30 min, before being concentrated under reduced pressure to yield the free amine intermediate as a brown solid, that was used without further purification.

Step 2: The material obtained in step 1 was suspended in anhydrous DCM (0.05 M), DIPEA (15 μL, 0.09 mmol, 3.0 eq.) was added, and a clear solution was observed. The mixture was then added dropwise at r.t. to a solution of X10 (11.6 mg, 0.043 mmol, 1.5 eq.) (pre-activated with COCl₂/DMF), and the resulting mixture was stirred at r.t. for 1 h. Complete turnover as indicated by TLC. The mixture was poured into 5 mL sat. aq. NaCl and 5 mL water was added. The mixture was extracted with DCM (4×10 mL). The combined organic layers were dried over Na₂SO₄ and concentrated under reduced pressure. Purification by FCC (Hex:EtOAc; 1:1 to 0:1) gave B10 as a colorless solid (21.6 mg, 0.022 mmol, 76%).

TLC R_{f}=0.59 (EA). HRMS (ESI): C₅₃H₅₃CIN₅O₉S₂: [M−H]⁻ calc. 1002.29787, found 1002.29730. ¹H-NMR (800 MHZ, CDCl₃) δ (ppm)=9.44 (s, 1H), 8.35 (s, 1H), 7.76 (d, J=8.1 Hz, 2H), 7.74-7.70 (m, 2H), 7.55 (s, 3H), 7.45 (s, 1H), 7.37 (d, J=18.8 Hz, 2H), 7.31-7.21 (m, 2H), 6.98 (s, 1H), 6.87 (s, 1H), 5.10 (s, 1H), 4.79 (d, J=10.2 Hz, 1H), 4.70-4.62 (m, 1H), 4.53-4.28 (m, 4H), 4.17 (d, J=29.1 Hz, 2H), 4.07 (s, 3H), 4.05-4.01 (m, 1H), 3.99-3.92 (m, 5H), 3.91 (s, 3H), 3.82-3.63 (m, 3H), 3.48 (t, J=10.9 Hz, 1H), 3.43-2.87 (m, 5H), 2.42 (s, 2H), 1.70 (d, J=43.0 Hz, 3H), 1.63-1.24 (m, 6H). ¹³C-NMR (201 MHZ, CDCl₃): δ (ppm)=160.5, 156.6, 150.4, 150.3, 147.8, 144.1, 141.5, 141.4, 140.7, 140.6, 139.0, 130.1, 129.9, 129.6, 128.0, 127.7, 127.3, 127.1, 125.8, 125.6, 125.2, 123.7, 122.9, 122.4, 120.0, 111.4, 106.8, 97.8, 97.7, 67.1, 66.6, 61.6, 61.3, 60.0, 56.4, 55.2, 47.4, 45.9, 43.5, 40.8, 39.1, 35.0, 34.3, 33.9, 29.7, 26.3, 24.8.

### (C) Disulfide-ADC Precursors—Examples of Compounds of Formula (V)

### General Protocol A: Assembly of 4-Formylphenylcarbamate Precursors

Step 1: Optionally substituted 4-hydroxy-benzaldehyde (1.0 equiv.) was dissolved in anhydrous DMF (0.05 M) and TEA (3.5 equiv.) was added. To the mixture was added dropwise at r.t. a solution of bis(pentafluorophenyl) carbonate (1.0 equiv.) in anhydrous DMF (0.2 M) and the resulting clear solution was stirred at r.t. for 30 min. A light purple colour was observed.

Step 2: Piperazine derivates (X6 or X11) (1.2 equiv.) was dissolved/suspended in anhydrous DMF (0.1 M) and optionally TEA (2.5 equiv.) was added. To this, the mixture obtained in step 1 was added dropwise at r.t. and the resulting solution was stirred at r.t. for further 2 h.

Step 3: To the mixture obtained in step 2 was added a solution of optionally substituted carboxylic acid (e.g., 6-(tert-butoxy)-6-oxohexanoic acid, 6-((tert-butoxycarbonyl)amino) hexanoic acid, etc.) (1.3 equiv.) in anhydrous DMF (0.2 M), followed by COMU (1.5 equiv.) and TEA (1.5 equiv.). The resulting mixture was stirred at r.t. for 3 h, was then diluted with EtOAc and washed with aq. NaHCO₃ (2×), aq. NH₄Cl (2×) and aq. NaCl (1×). The combined organic fractions were dried over MgSO4 and concentrated under reduced pressure. Purification by FCC (isohexane:EtOAc, 9:1 to 0:1) gave the products were obtained as colourless solids.

### General Protocol B: Pre-Activation of Piperazine Precursors

Step 1:4-formylphenylcarbamate precursors (1.0 equiv.) were suspended in MeOH:H₂O (1:1, 0.05 M) and acetic acid (150 equiv.) was added. A clear solution was observed. To this mixture was added at r.t. a solution of NaCNBH₃ (4.0 equiv.) in MeOH (0.2 M) and the resulting mixture was stirred at r.t. for 2 h. The mixture was poured into a mixture of DCM and aq. NaHCO₃ (1:1, excess) and gas formation was observed. The aq. layer was extracted with DCM (2×), and the combined organic layers were washed with aq. Na₂CO₃ (1×) and aq. NaCl (1×), was then dried over MgSO₄ and concentrated under reduced pressure.

Step 2: The material obtained in step 1 was dissolved in anhydrous DCM (0.05 M) and TEA (2.0 equiv.) was added. To the resulting mixture was added a solution of 4-nitrophenyl chloroformate in anhydrous DCM (0.2 M) at r.t . . . . Immediate colour change to light yellow/green and gas formation (HNEt₃Cl) was observed. The resulting yellow solution was stirred at r.t. for 1 h, before being poured in a mixture of DCM and aq. NH₄Cl (1:1, excess). The aq. layer was extracted with DCM (2×), and the combined organic layers were dried over MgSO₄ and concentrated under reduced pressure. Purification was achieved by FCC (isohexane:EtOAc, 9:1 to 0:1) or preparative HPLC (H₂O/MeCN, 0.1% FA) and the products were obtained as colourless oils.

### 4-formylphenyl 4-(6-(tert-butoxy)-6-oxohexanoyl)hexahydro-[1,2]dithiino[4,5-b]pyrazine-1(2H)-carboxylate (trans) (X13)

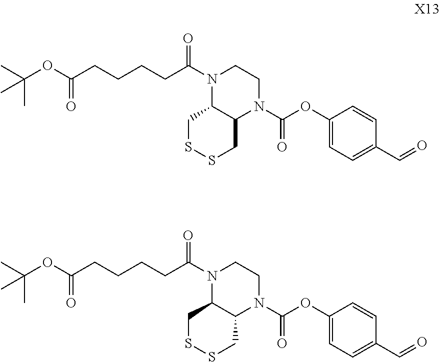

X13 (102 mg, 0.20 mmol, 35%) was prepared from 4-hydroxybenzaldehyde (70 mg, 0.57 mmol), bis(pentafluorophenyl) carbonate (226 mg, 0.57 mmol) and TEA (0.28 mL, 2.0 mmol) (step 1); X2 (·2 HCl, ·1.1 NaCl, 71 w %) (241 mg, 0.69 mmol) and TEA (0.44 mL, 0.32 mmol) (step 2); and 6-(tert-butoxy)-6-oxohexanoic acid (197 mg, 0.97 mmol), COMU (417 mg, 0.97 mmol), TEA (0.14 mL, 0.97 mmol) (step 3) according to general protocol A.

TLC R_{f}=0.63 (isohexane:EtOAc, 1:3). HRMS (ESI): C₂₄H₃₂N₂NaO₆S₂⁺: [M+Na]⁺ calc. m/z 531.15940, found 531.15953. ¹H-NMR (400 MHZ, CDCl₃): δ (ppm)=9.98 (s, 1H), 7.90 (d, J=8.6 Hz, 2H), 7.25 (d, J=8.0 Hz, 2H), 3.90 (d, J=144.9 Hz, 6H), 3.29 (d, J=12.5 Hz, 2H), 3.23-2.87 (m, 2H), 2.45-2.26 (m, 2H), 2.23 (t, J=7.0 Hz, 2H), 1.73-1.57 (m, 4H), 1.42 (s, 9H). ¹³C-NMR (101 MHz, CDCl₃): δ (ppm)=191.0, 172.8, 155.4, 134.0, 131.3, 122.4, 80.4, 60.0, 39.9, 35.2, 34.1, 28.2, 24.8, 24.6.

### 4-((((4-nitrophenoxy)carbonyl)oxy)methyl)phenyl 4-(6-(tert-butoxy)-6-oxohexanoyl) hexahydro-[1,2]dithiino[4,5-b]pyrazine-1(2H)-carboxylate (trans) (B14)

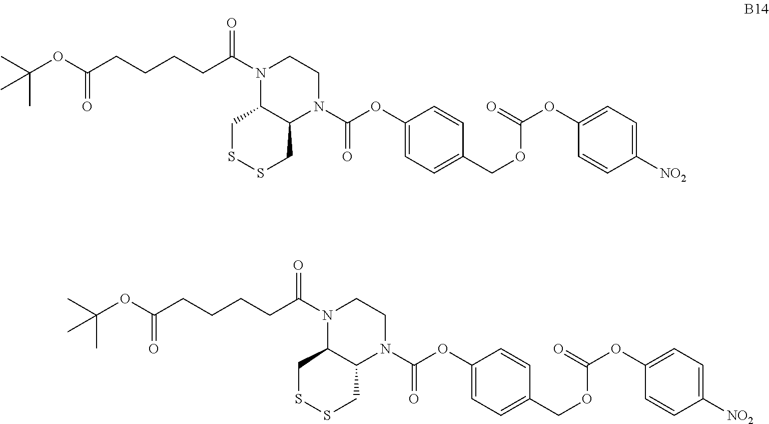 [SMILES: CC(C)(C)OC(=O)CCCCC(=O)N1CCN(C(=O)Oc2ccc(COC(=O)Oc3ccc([N+](=O)[O-])cc3)cc2)[C@H]2CSSC[C@@H]21 ; CC(C)(C)OC(=O)CCCCC(=O)N1CCN(C(=O)Oc2ccc(COC(=O)Oc3ccc([N+](=O)[O-])cc3)cc2)[C@@H]2CSSC[C@H]21]

B16 (70.0 mg, 0.104 mmol, 53%) was prepared from X13 (273 mg, 0.652 mmol), NaCNBH₃ (164 mg, 2.61 mmol), acetic acid (5.6 mL, 98 mmol) (step 1); 4-nitrophenyl chloroformate (131 mg, 0.652 mmol) and TEA (181 μL, 1.3 mmol) (step 2) according to general protocol B.

TLC R_{f}=0.62 (isohexane:EtOAc, 1:2). HRMS (ESI): C₃₁H₃₇N₃NaO10S2+: [M+Na]⁺ calc. m/z 698.18126, found 698.18160. ¹H-NMR (400 MHZ, CDCl₃): δ (ppm)=8.4-8.2 (m, 2H), 7.5 (d, J=8.5 Hz, 2H), 7.4-7.3 (m, 2H), 7.1 (d, J=7.9 Hz, 2H), 5.3 (s, 2H), 4.4-3.5 (m, 7H), 3.3 (d, J=12.7 Hz, 1H), 3.2 (s, 1H), 2.5-2.3 (m, 2H), 2.2 (t, J=6.9 Hz, 2H), 1.8-1.5 (m, 6H), 1.4 (s, 9H). ¹³C-NMR (101 MHZ, CDCl₃): δ (ppm)=172.9, 155.6, 152.5, 151.3, 145.6, 131.9, 130.2, 125.4, 122.2, 121.9, 80.4, 70.4, 59.4, 39.3, 35.3, 33.1, 28.3, 24.8, 24.6.

### 4-formylphenyl 4-(6-(tert-butoxy)-6-oxohexanoyl)hexahydro-[1,2]dithiino[4,5-b]pyrazine-1(2H)-carboxylate (cis) (X14)

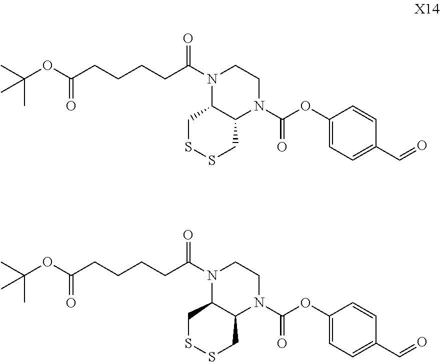 [SMILES: CC(C)(C)OC(=O)CCCCC(=O)N1CCN(C(=O)Oc2ccc(C=O)cc2)[C@@H]2CSSC[C@@H]21 ; CC(C)(C)OC(=O)CCCCC(=O)N1CCN(C(=O)Oc2ccc(C=O)cc2)[C@H]2CSSC[C@H]21]

X14 (38 mg, 0.075 mmol, 40%) was prepared from 4-hydroxybenzaldehyde (23 mg, 0.19 mmol), bis(pentafluorophenyl) carbonate (74 mg, 0.19 mmol) and TEA (91 μL, 0.66 mmol) (step 1); X6 (·2 HCl, ·2.5 NaCl, 60 w %) (117 mg, 0.28 mmol) and TEA (44 μL, 0.32 mmol) (step 2); and 6-(tert-butoxy)-6-oxohexanoic acid (76 mg, 0.38 mmol), COMU (161 mg, 0.38 mmol), TEA (52 μL, 0.38 mmol) (step 3) according to general protocol A.

TLC R_{f}=0.61 (isohexane:EtOAc, 1:3). HRMS (ESI): C₂₄H₃₂N₂NaO₆S₂⁺: [M+Na]⁺ calc. m/z 531.15940, found 531.15966. ¹H-NMR (400 MHZ, CDCl₃): δ (ppm)=9.98 (s, 1H), 7.91 (d, J=8.5 Hz, 2H), 7.30 (d, J=8.5 Hz, 2H), 4.71 (s, 1H), 4.39 (d, J=2.8 Hz, 1H), 4.20 (s, 1H), 3.97-3.83 (m, 2H), 3.71 (d, J=14.6 Hz, 2H), 3.50 (dd, J=14.0, 9.0 Hz, 1H), 3.16 (dd, J=14.4, 2.8 Hz, 1H), 2.96 (d, J=11.2 Hz, 1H), 2.36 (q, J=6.7 Hz, 2H), 2.25 (t, J=6.9 Hz, 2H), 1.76-1.55 (m, 4H), 1.43 (s, 9H). ¹³C-NMR (101 MHZ, CDCl₃): δ (ppm)=191.0, 172.9, 172.5, 155.6, 153.0, 134.0, 131.3, 122.4, 80.4, 51.7, 49.8, 42.6, 42.3, 36.4, 35.3, 33.4, 28.2, 24.8, 24.4.

### 4-((((4-nitrophenoxy)carbonyl)oxy)methyl)phenyl 4-(6-(tert-butoxy)-6-oxohexanoyl)hexahydro-[1,2]dithiino[4,5-b]pyrazine-1(2H)-carboxylate (cis) (B15)

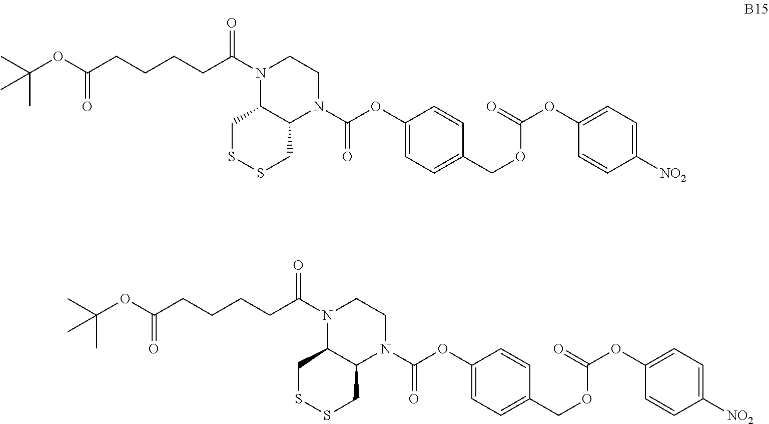 [SMILES: CC(C)(C)OC(=O)CCCCC(=O)N1CCN(C(=O)Oc2ccc(COC(=O)Oc3ccc([N+](=O)[O-])cc3)cc2)[C@@H]2CSSC[C@@H]21 ; CC(C)(C)OC(=O)CCCCC(=O)N1CCN(C(=O)Oc2ccc(COC(=O)Oc3ccc([N+](=O)[O-])cc3)cc2)[C@H]2CSSC[C@H]21]

B17 (16.5 mg, 0.025 mmol, 33%) was prepared from X14 (38.0 mg, 0.075 mmol), NaCNBH₃ (18.8 mg, 0.30 mmol), acetic acid (0.6 mL, 11.2 mmol) (step 1); 4-nitrophenyl chloroformate (15.1 mg, 0.075 mmol) and TEA (21 μL, 0.15 mmol) (step 2) according to general protocol B.

TLC R_{f}=0.77 (isohexane:EtOAc, 1:2). HRMS (ESI): C₃₁H₃₇N₃NaO₁₀S₂⁺: [M+Na]⁺ calc. m/z 698.18126, found 698.18175. ¹H-NMR (400 MHZ, CDCl₃): δ (ppm)=8.28 (d, J=9.1 Hz, 2H), 7.47 (d, J=8.4 Hz, 2H), 7.38 (d, J=9.1 Hz, 2H), 7.17 (d, J=8.4 Hz, 2H), 5.28 (s, 2H), 4.72 (s, 1H), 4.47-4.33 (m, 1H), 4.21 (s, 1H), 3.87 (d, J=7.7 Hz, 2H), 3.74 (s, 2H), 3.50 (dd, J=13.7, 9.1 Hz, 1H), 3.17 (d, J=11.9 Hz, 1H), 2.97 (d, J=11.9 Hz, 1H), 2.36 (q, J=6.6 Hz, 2H), 2.26 (t, J=6.9 Hz, 2H), 1.76-1.61 (m, 4H), 1.44 (s, 9H). ¹³C-NMR (101 MHZ, CDCl₃): δ (ppm)=172.9, 172.5, 155.6, 153.8, 152.6, 151.5, 145.6, 131.9, 130.2, 125.5, 122.2, 121.9, 80.4, 70.4, 51.6, 42.7, 42.2, 38.8, 36.4, 35.3, 33.4, 30.0, 28.3, 24.8, 24.4.

### 4-formylphenyl 4-(6-((tert-butoxycarbonyl)amino)hexanoyl)hexahydro-[1,2]dithiino[4,5-b]pyrazine-1(2H)-carboxylate (trans) (X15)

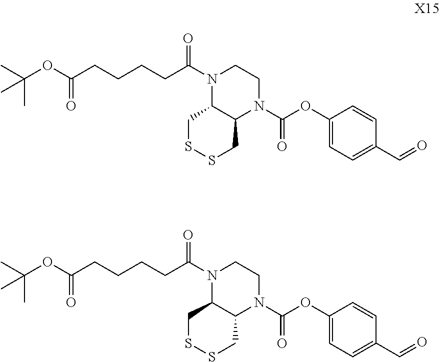 [SMILES: CC(C)(C)OC(=O)CCCCC(=O)N1CCN(C(=O)Oc2ccc(C=O)cc2)[C@H]2CSSC[C@@H]21 ; CC(C)(C)OC(=O)CCCCC(=O)N1CCN(C(=O)Oc2ccc(C=O)cc2)[C@@H]2CSSC[C@H]21]

X15 (126 mg, 0.23 mmol, 62%) was prepared from 4-hydroxybenzaldehyde (46 mg, 0.38 mmol), bis(pentafluorophenyl) carbonate (148 mg, 0.38 mmol), TEA (0.2 mL, 1.5 mmol) (step 1); X2 (2 HCl, -1.1 NaCl, 71 w %) (198 mg, 0.56 mmol), TEA (88 μL, 0.64 mmol) (step 2); 6-((tert-butoxycarbonyl)amino) hexanoic acid (174 mg, 0.75 mmol), COMU (321 mg, 0.75 mmol), TEA (0.1 mL, 0.75 mmol) (step 3) according to general protocol A.

TLC R_{f}=0.32 (isohexane:EtOAc, 1:2). HRMS (ESI): C₂₅H₃₅N₃NaO₆S₂⁺: [M+Na]⁺ calc. m/z 560.18595, found 560.18618. ¹H-NMR (400 MHZ, CDCl₃): δ (ppm)=9.98 (s, 1H), 7.90 (d, J=8.5 Hz, 2H), 7.25 (d, J=8.8 Hz, 2H), 4.57 (s, 1H), 4.41-3.50 (m, 7H), 3.29 (d, J=12.9 Hz, 1H), 3.22-2.98 (m, 4H), 2.32 (dd, J=15.7, 7.8 Hz, 2H), 1.75-1.55 (m, 2H), 1.51-1.45 (m, 2H), 1.42 (s, 9H), 1.39-1.29 (m, 2H). ¹³C-NMR (101 MHZ, CDCl₃): δ (ppm)=191.0, 156.1, 155.4, 134.0, 131.3, 122.4, 79.2, 59.9, 43.1, 40.3, 39.2, 34.1, 29.9, 28.5, 26.5, 24.8.

### 4-((((4-nitrophenoxy)carbonyl)oxy)methyl)phenyl 4-(6-((tert-butoxycarbonyl)amino)hexanoyl) hexahydro-[1,2]dithiino[4,5-b]pyrazine-1(2H)-carboxylate (trans) (B16)

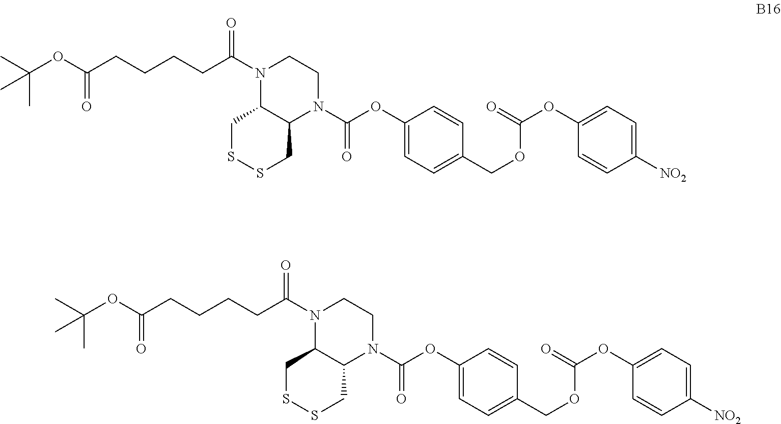 [SMILES: CC(C)(C)OC(=O)CCCCC(=O)N1CCN(C(=O)Oc2ccc(COC(=O)Oc3ccc([N+](=O)[O-])cc3)cc2)[C@H]2CSSC[C@@H]21 ; CC(C)(C)OC(=O)CCCCC(=O)N1CCN(C(=O)Oc2ccc(COC(=O)Oc3ccc([N+](=O)[O-])cc3)cc2)[C@@H]2CSSC[C@H]21]

B18 (60.5 mg, 0.086 mmol, 40%) was prepared from X15 (116.0 mg, 0.216 mmol), NaCNBH₃ (54 mg, 0.86 mmol) and acetic acid (1.8 mL, 32.3 mmol) (step 1); 4-nitrophenyl chloroformate (43.5 mg, 0.216 mmol), TEA (60 μL, 0.43 mmol) (step 2) according to general protocol B.

TLC R_{f}=0.37 (isohexane:EtOAc, 1:2). HRMS (ESI): C₂₇H₃₃N₄O₈S₂⁺: [M-Boc+2H]⁺ calc. m/z 605.17343, found 605.17399. ¹H-NMR (400 MHZ, CDCl₃): δ (ppm)=8.27 (d, J=9.2 Hz, 2H), 7.45 (d, J=8.5 Hz, 2H), 7.38 (d, J=9.2 Hz, 2H), 7.11 (d, J=7.6 Hz, 2H), 5.27 (s, 2H), 4.57 (s, 1H), 4.42-3.44 (m, 8H), 3.29 (d, J=12.6 Hz, 1H), 3.23-3.00 (m, 3H), 2.44-2.21 (m, 2H), 1.76-1.57 (m, 2H), 1.52-1.46 (m, 2H), 1.43 (s, 9H), 1.35 (q, J=8.6 Hz, 2H). 13C-NMR (101 MHZ, CDCl₃): δ (ppm)=173.1, 156.1, 155.6, 152.5, 151.3, 145.6, 131.9, 130.2, 125.4, 122.2, 121.9, 79.2, 70.3, 59.9, 40.4, 34.0, 30.0, 28.6, 26.5, 24.8.

### 4-formylphenyl 4-(6-((tert-butoxycarbonyl)amino)hexanoyl)hexahydro-[1,2]dithiino[4,5-b]pyrazine-1(2H)-carboxylate (cis) (X16)

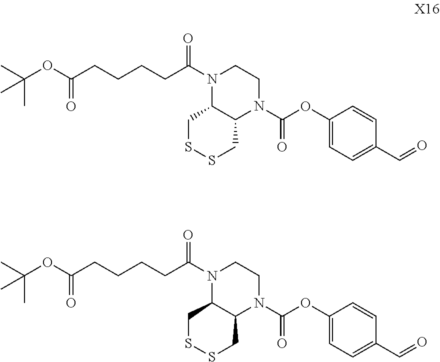 [SMILES: CC(C)(C)OC(=O)CCCCC(=O)N1CCN(C(=O)Oc2ccc(C=O)cc2)[C@@H]2CSSC[C@@H]21 ; CC(C)(C)OC(=O)CCCCC(=O)N1CCN(C(=O)Oc2ccc(C=O)cc2)[C@H]2CSSC[C@H]21]

X16 (39.0 mg, 0.073 mmol, 39%) was prepared from 4-hydroxybenzaldehyde (22.9 mg, 0.188 mmol), bis(pentafluorophenyl) carbonate (73.9 mg, 0.19 mmol), TEA (0.2 mL, 1.5 mmol) (step 1); X6 (·2 HCl, 2.5 NaCl, 60 w %) (117 mg, 0.28 mmol), TEA (44 μL, 0.32 mmol) (step 2); 6-((tert-butoxycarbonyl)amino) hexanoic acid (86.7 mg, 0.38 mmol), COMU (161 mg, 0.38 mmol), TEA (52 μL, 0.38 mmol) (step 3) according to general protocol A.

TLC R_{f}=0.33 (isohexane:EtOAc, 1:2). HRMS (ESI): C₂₅H₃₅N₃NaO₆S₂⁺: [M-Boc+Na]⁺ calc. m/z 460.13352, found 460.13947. HPLC-MS: t_{R}=6.9 min (10 min run, H₂O:MeCN, from 10%→100% MeCN, 0.1% formic acid); ESI-MS: C₂₀H₂₇N₃NaO₄S₂⁺ [M-Boc+H+Na]⁺ calc. m/z 460.1, found 460.1.

### 4-((((4-nitrophenoxy)carbonyl)oxy)methyl)phenyl 4-(6-((tert-butoxycarbonyl)amino)hexanoyl) hexahydro-[1,2]dithiino[4,5-b]pyrazine-1(2H)-carboxylate (cis) (B17)

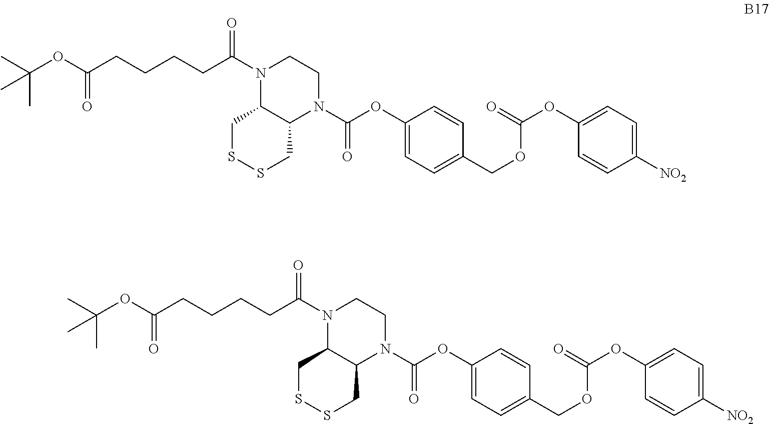 [SMILES: CC(C)(C)OC(=O)CCCCC(=O)N1CCN(C(=O)Oc2ccc(COC(=O)Oc3ccc([N+](=O)[O-])cc3)cc2)[C@@H]2CSSC[C@@H]21 ; CC(C)(C)OC(=O)CCCCC(=O)N1CCN(C(=O)Oc2ccc(COC(=O)Oc3ccc([N+](=O)[O-])cc3)cc2)[C@H]2CSSC[C@H]21]

B19 (15.0 mg, 0.021 mmol, 43%) was prepared from X16 (33.0 mg, 0.049 mmol), NaCNBH₃ (12.3 mg, 0.20 mmol) and acetic acid (0.4 mL, 7.4 mmol) (step 1); 4-nitrophenyl chloroformate (9.9 mg, 0.049 mmol) and TEA (14 μL, 0.10 mmol) (step 2) according to general protocol B.

TLC R_{f}=0.41 (isohexane:EtOAc, 1:2). HRMS (ESI): C₃₂H₄₀N₄NaO₈S₂⁺: [M+Na]⁺ calc. m/z 727.20781, found 727.20870. ¹H-NMR (400 MHZ, CDCl₃): δ (ppm)=8.31-8.24 (m, 2H), 7.47 (d, J=8.4 Hz, 2H), 7.40-7.35 (m, 2H), 7.16 (d, J=8.4 Hz, 2H), 5.28 (s, 2H), 4.72 (s, 1H), 4.57 (s, 1H), 4.44-4.34 (m, 1H), 4.21 (s, 1H), 3.95-3.82 (m, 2H), 3.73 (s, 2H), 3.51 (dd, J=13.6, 9.1 Hz, 1H), 3.25-3.06 (m, 3H), 2.97 (d, J=12.7 Hz, 1H), 2.34 (hept, J=7.8 Hz, 2H), 1.68 (p, J=7.4 Hz, 2H), 1.52 (p, J=7.2 Hz, 2H), 1.44 (s, 9H), 1.38 (p, J=7.7, 7.0 Hz, 2H). ¹³C-NMR (101 MHZ, CDCl₃): δ (ppm)=172.7, 156.2, 155.6, 153.8, 152.5, 151.4, 145.6, 131.9, 130.2, 125.4, 122.2, 121.9, 79.3, 70.4, 51.6, 49.8, 42.7, 42.3, 40.5, 38.3, 36.4, 33.6, 30.0, 28.6, 26.6, 24.6.

### 5. Biological Testing

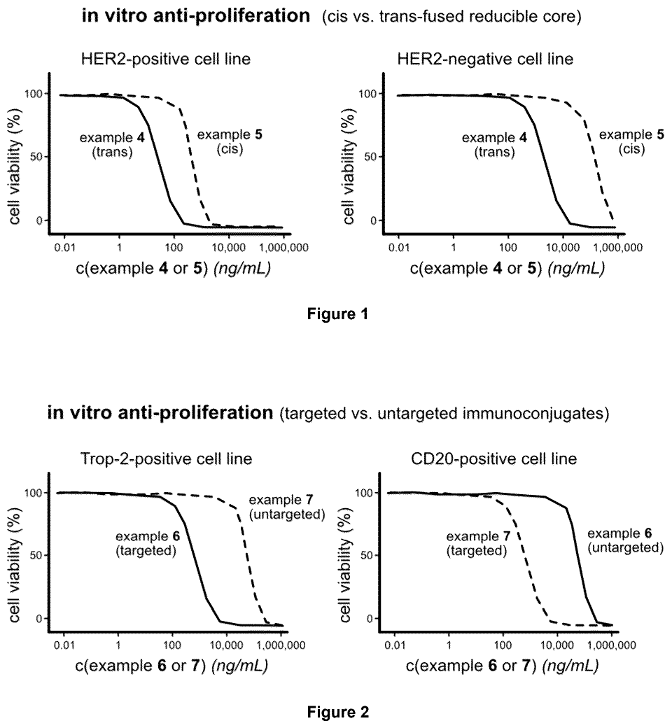

### 5.1 Cellular Anti-Proliferation Assays (See FIGS. 1 and 2)

Cells (HER2-positive cell line, HER2-negative cell line, Trop2-positive cell line, or CD20-positive cell line) are cultured under standard conditions and antigen density of the cell lines is tested using flow cytometry-based measurements. For anti-proliferative experiments cells are left to adhere for 15 h and molecules of the invention 4, 5, 6 or 7 (diluted from 10 mg/mL in DPBS (Dulbecco's PBS (Phosphate Buffered Saline))) are added. Cells are incubated under standard conditions for 7 days, then 0.20 volume equivalents of a solution of resazurin in DPBS (0.15 mg/mL) are added and incubated for 3 hours, and then fluorescence (ex 544 nm, em 590 nm) is detected on a fluorescence platereader. Cell viability (%) is calculated from the fluorescence values relative to a control experiment with no 4, 5, 6 or 7 added.

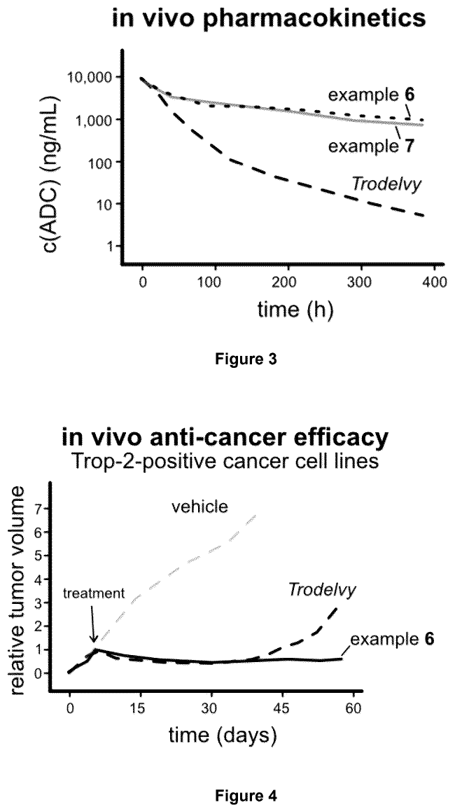

### 5.2 In Vivo Mouse Assays (See FIGS. 3 and 4)

Mice are maintained in individually ventilated cages at constant temperature (22±2° C., air-conditioned) and humidity (45-65%), under optimum hygienic conditions with 10-15 air changes per hour. A cycle of 12 h artificial fluorescent lightning and 12 h darkness is applied and animal behavior is monitored daily throughout the study. The mice receive food and water ad libitum.

### Pharmacokinetic Study

To study the in vivo disposition of molecules of the invention mice are treated once with 6, 7 or commercial Trodelvy® at a dose of 7 mg/kg by intravenous injection. Plasma samples are collected at the following time points: predose, postdose: 2 min, 6 h, 24 h, 48 h, 72 h, 168 h, 240 h, and 380 h. All samples are analyzed using an MS-based analytical method for quantification of the intact immunoconjugates and of the free molecular cargo in the mouse plasma.

### Anti-Cancer Efficacy Study—Trop-2-Positive Pancreatic Cancer Model

On day zero, 1×10⁵ Trop-2-positive pancreatic cancer cells in 100 μL DPBS are implanted into the left mammary fat pad of all mice. After reaching a mean tumour volume of 200 mm³, animals are randomized and the mice are treated with 6, or else with commercial Trodelvy®, at 3 mg/kg from immunoconjugates formulated in DPBS, with a single-dose intravenous injection, while the control group receive DPBS vehicle. The study is continued while animals are maintained in good condition, up to a limit of day 57, during which time tumour size is measured twice weekly.

1. A compound having the formula (I)
T-K-A-L-B (I)
wherein
T is a targeting unit that binds to a target biomolecule of interest;
K is a linker that is composed of —W-D⁴-D³-D²-D¹-, wherein W is bound to T and D¹ is bound to the N atom of A;
A is a structure of formula (I) 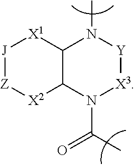
L is a bond or a self-immolative spacer which is bound to the C(═O) of A;
B is —O-A¹, —N(A²)-A³, —S-A⁴, or —O(O)C-A⁵;
J and Z are independently selected from S or Se such that either J is Se and Z is S, or J is S and Z is Se, or J and Z are both S;
X¹ is —(CR^{d}₂)ₘ—;
X² is —(CR^{e}₂)ₙ—;
X³ is —CR^{f}₂—;
Y is —(CR^{g}₂)ₚ—;
A¹ is selected such that A¹-OH is a therapeutic agent which contains an —OH moiety;
A² is selected such that A²-NH-A³ is a therapeutic agent which contains an —NH₂ or —NH-moiety;
A³ is selected such that A²-NH-A³ is a therapeutic agent which contains an —NH₂ or —NH-moiety;
A⁴ is selected such that A⁴-SH is a therapeutic agent which contains an —SH moiety;
A⁵ is selected such that A⁵-COOH is a therapeutic agent which contains an —COOH moiety;
D¹ is selected from —CH₂—, —CH═CH—, —C≡C—, —C(O)—, —C(O)—O—, —C(O)—NH—, —C(O)—S—, —S(O)—, —S(O)₂—, —P(O)(O-E²)-, —S(O)₂—NH—, —P(O)(O-E²)—O—, and —P(O)(O-E²)—NH—;
D² is either a bond or a spacer;
D³ is a bond or an adapting unit;
D⁴ is either a bond or a spacer;
R^{d} is independently selected from —H and —C₁₋₄-alkyl;
R^{e} is independently selected from —H and —C₁₋₄-alkyl;
R^{r} is independently selected from —H and —C₁₋₄-alkyl;
R^{g} is independently selected from —H and —C₁₋₄-alkyl;
m is 0, 1 or 2;
n is 1 or 2, provided that m+n is 2 or 3;
p is 1, or 2;
W is a bioconjugation attachment that results from reaction of a functional group with the targeting unit T; and
E² is selected from —H, —C₁₋₄-alkyl, —C₁₋₄-alkyl-SO₃H, —C₁₋₄-alkyl-OPO₃H₂—C(O)-Me, —C(O)—O—C₁₋₄-alkyl, —C(O)—NH—C₁₋₄-alkyl, —S(O)₂—C₁₋₄-alkyl, —(CH₂—CH₂—O)₁₋₈₀—CH₂—CH₂—OH, and —CH₂—CH₂—N(CH₂—CH₂—OH)₂;
or a stereoisomer, racemic mixture, pharmaceutically acceptable salt, ester, hydrate, or solvate thereof.

2. The compound according to claim 1, wherein
T is selected from a monoclonal antibody (mAb), polyclonal antibody, antibody fragment, Fab, Fab′, Fab′-SH, Fv, single chain Fv, diabody, linear antibody, bispecific antibody, multispecific antibody, chimeric antibody, humanized antibody, human antibody, fusion protein, nanobody, fusion protein comprising the antigen-binding portion of an antibody, an antibody mimetic, functional fragment, antigen-binding antibody fragment, antigen-binding region, Fynomer®, antibody mimetic, affibody, adnectin, anticalin, DARPin, avimer, nanofitin, affilin, Kunitz domain peptide, trispecific binding molecule, or probody. Preferably, T is monoclonal antibody (mAb), human or humanized antibody, or an antibody fragment. More preferably, T is a monoclonal antibody (mAb).

3. The compound according to claim 1, wherein L is a bond.

4. The compound according to claim 1, wherein the compound having the formula (I) is selected from: 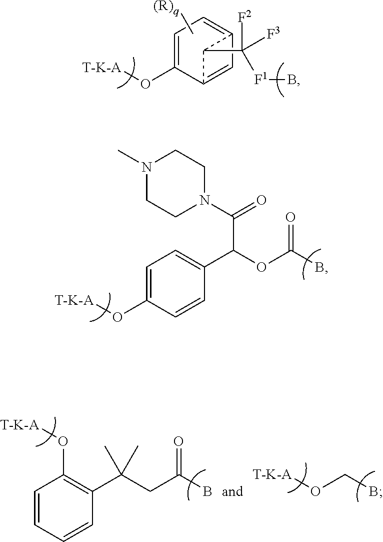
wherein
—F¹—B is selected from 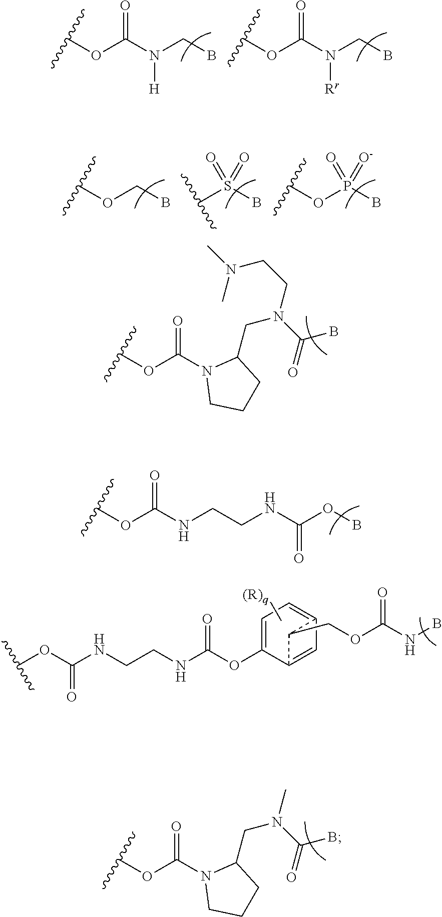
F² is selected from —H, —CF₃, and —C₁₋₄-alkyl;
F³ is selected from —H, —CF₃, and —C₁₋₄-alkyl;
R is independently selected from halogen, —O(R^{r}), —N(R^{s})(R^{t}), —NO₂, —CN, and a heterocyclic group selected from azetidinyl, pyrrolidinyl, piperidinyl and morpholino, wherein the heterocyclic group is attached to the phenyl ring via the N atom;
q is 0, 1, 2, 3 or 4;
R^{r} is selected from —H, —C₁₋₄-alkyl and —(C₂₋₄-alkylene)-O—(C₁₋₄-alkyl);
R^{s} is selected from —H, —C(O)—C₁₋₄-alkyl and —C₁₋₄-alkyl;
R^{t} is selected from —H, —C(O)—C₁₋₄-alkyl and —C₁₋₄-alkyl; and
T, K, A and B are as defined claim 1.

5. The compound according to claim 1, wherein the compound having the formula (I) is selected from: 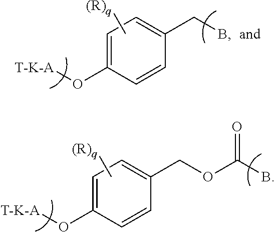
wherein
R is independently selected from halogen, —O(R^{r}), —N(R^{s})(R^{t}), —NO₂, —CN, and a heterocyclic group selected from azetidinyl, pyrrolidinyl, piperidinyl and morpholino, wherein the heterocyclic group is attached to the phenyl ring via the N atom;
q is 0, 1, 2, 3 or 4;
and
T, K, A and B are as defined claim 1.

6. The compound according to claim 1, wherein X¹ and X² are —CH₂—.

7. The compound according to claim 1, wherein
J and Z are both S.

8. The compound according to claim 1, wherein
D² is selected from —(CH₂)₁₋₁₆₀— and —(CH₂—CH₂—O)₁₋₈₀—; and/or
D³ is selected from 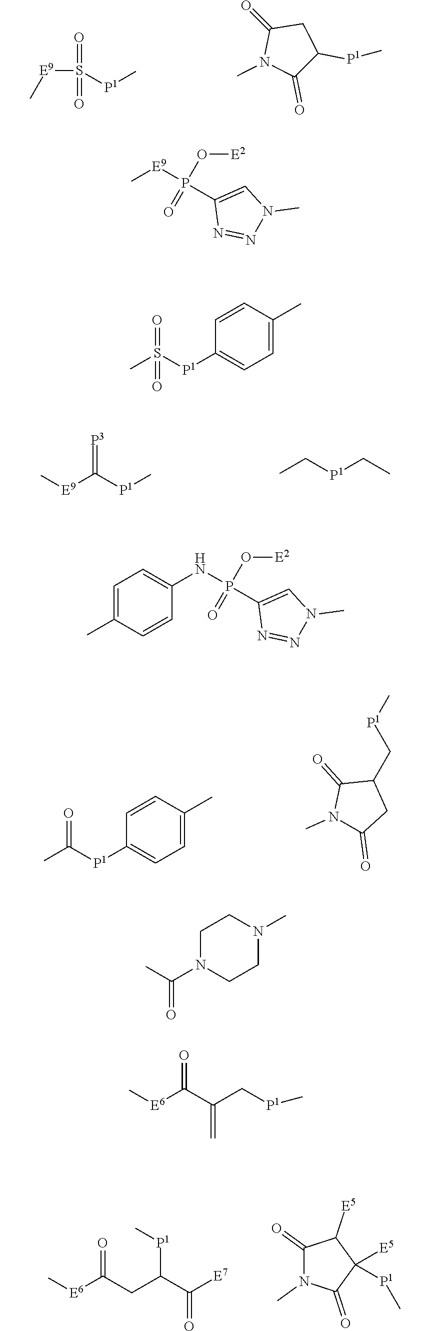 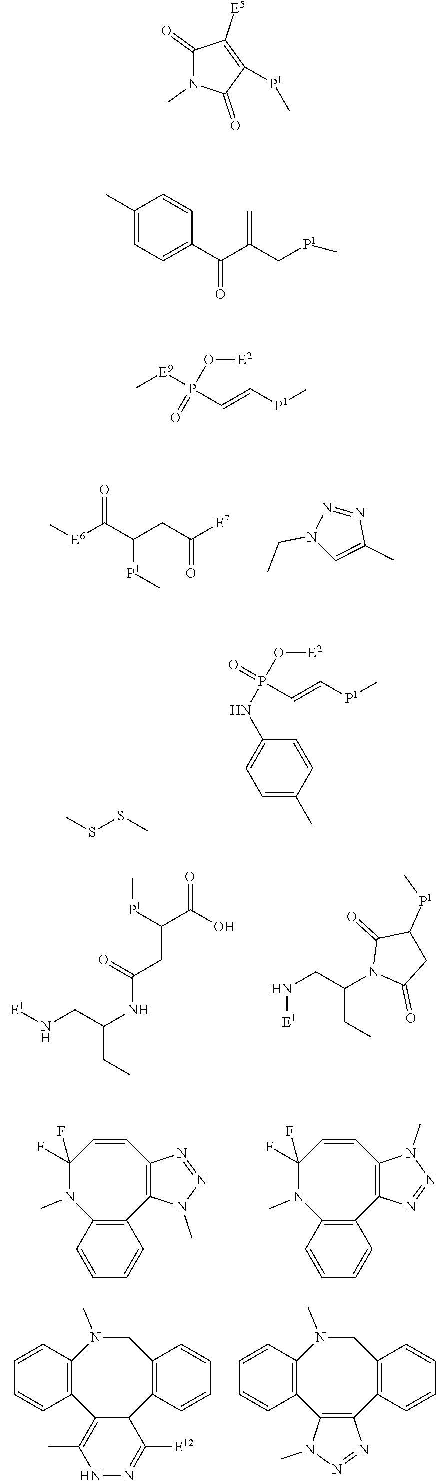 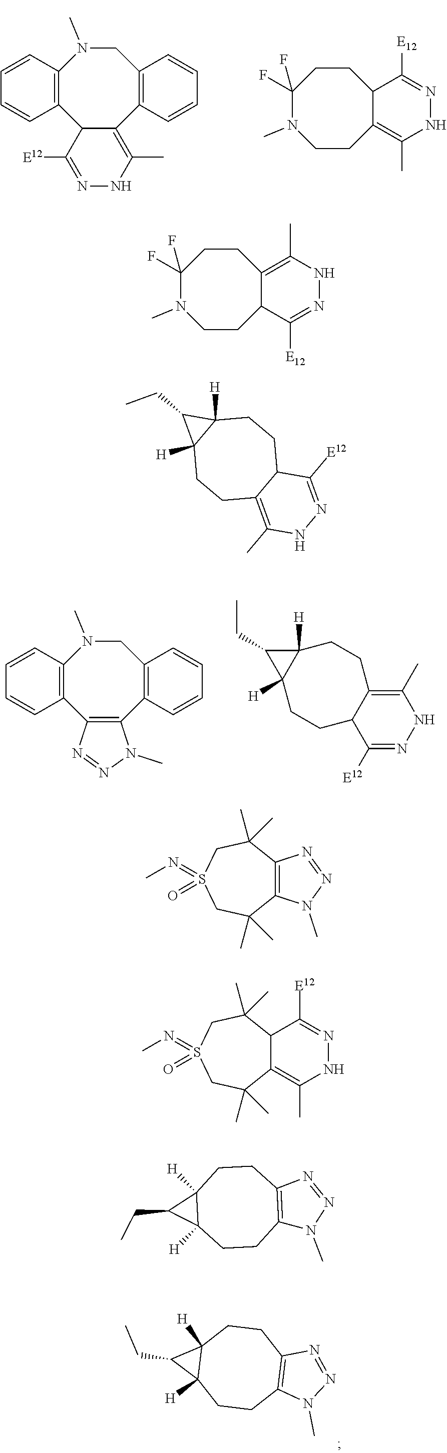
wherein either the dark grey oval represents D² and the light grey oval represents D⁴, or the light grey oval represents D² and the dark grey oval represents D⁴.
E² is selected from —H, —C₁₋₄-alkyl, —C₁₋₄-alkyl-SOH, —C₁₋₄-alkyl-OPO₃H₂; —C(O)-Me, —C(O)—O—C₁₋₄-alkyl, —C(O)—NH—C₁₋₄-alkyl, —S(O)₂—C₁₋₄-alkyl, —(CH₂—CH₂—O)₁₋₈₀ CH₂—CH₂—OH, and —CH₂—CH₂—N(CH₂—CH₂—OH)₂;
E⁵ is selected from —F, —Cl, —Br, —I, —OMs, and —OTs;
E⁶ is selected from —O—, —NH—, and —S—;
E⁷ is selected from an aromatic or heteroaromatic residue, preferably -Ph;
E⁹ is selected from —O—, —NH—, —N(C₁₋₄-alkyl)-, —CH₂—, and -1,2,3-triazolyl-;
E¹² is selected from —H, -Me, -Ph, -2-pyridinyl, and -5-pyrimidinyl;
P¹ is selected from —O—, —NH—, —N(E²)-, —S—, and —Se—; and
P³ is selected from ═O, ═NH, ═N(E²), ═S, and ═Se.

9. The compound according to claim 1, wherein
the compound is selected from 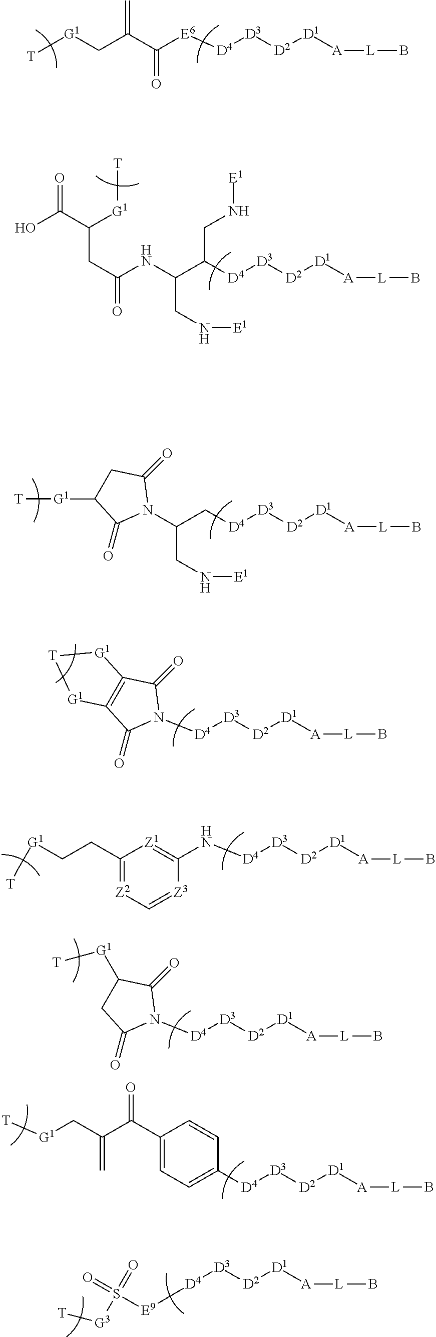 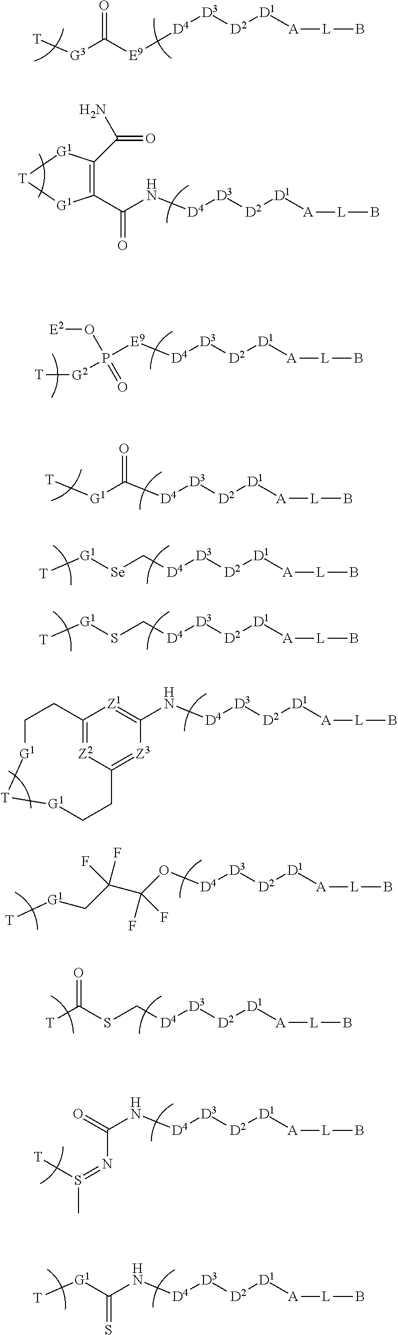 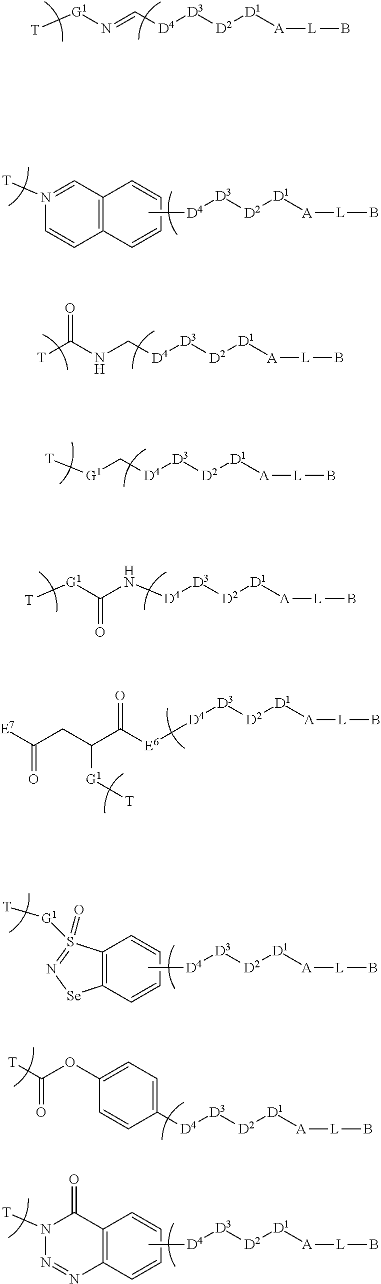 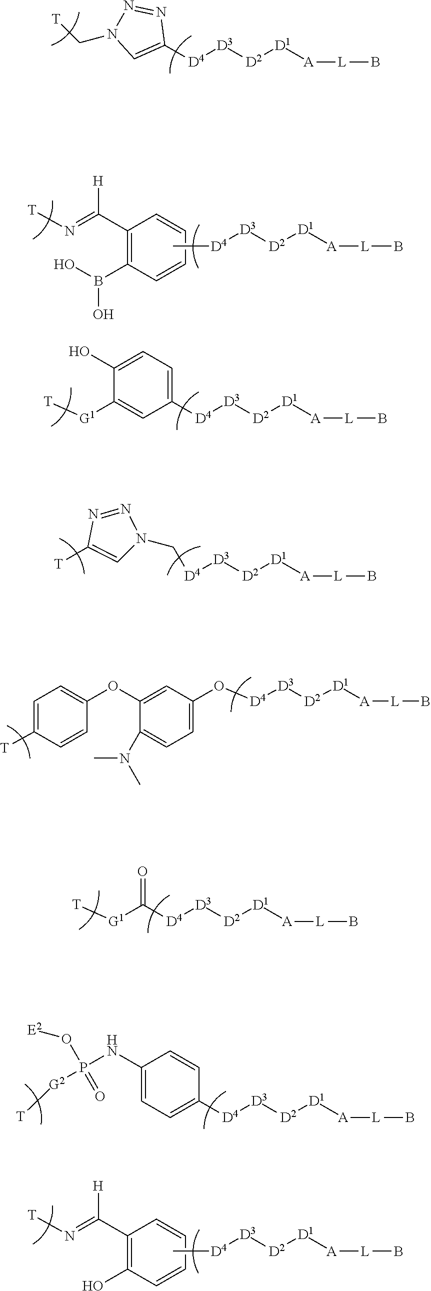 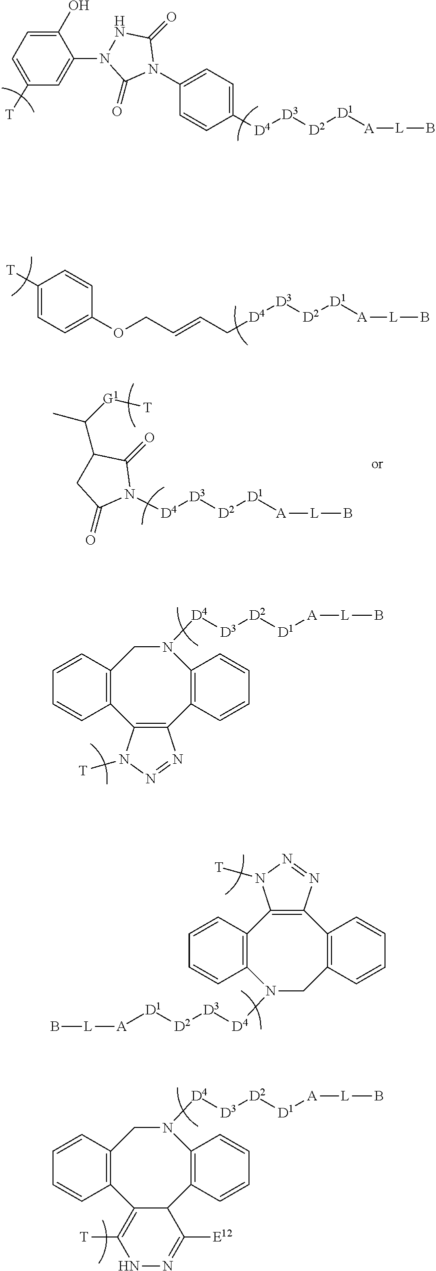 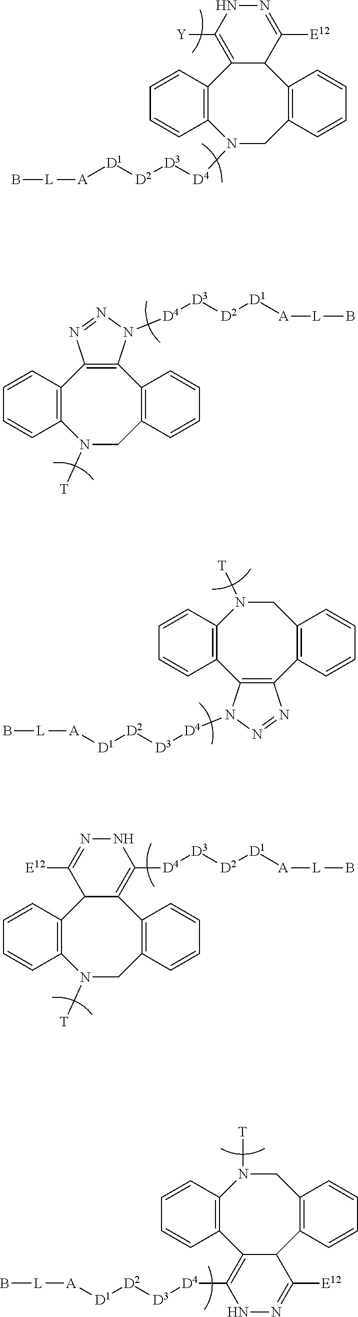 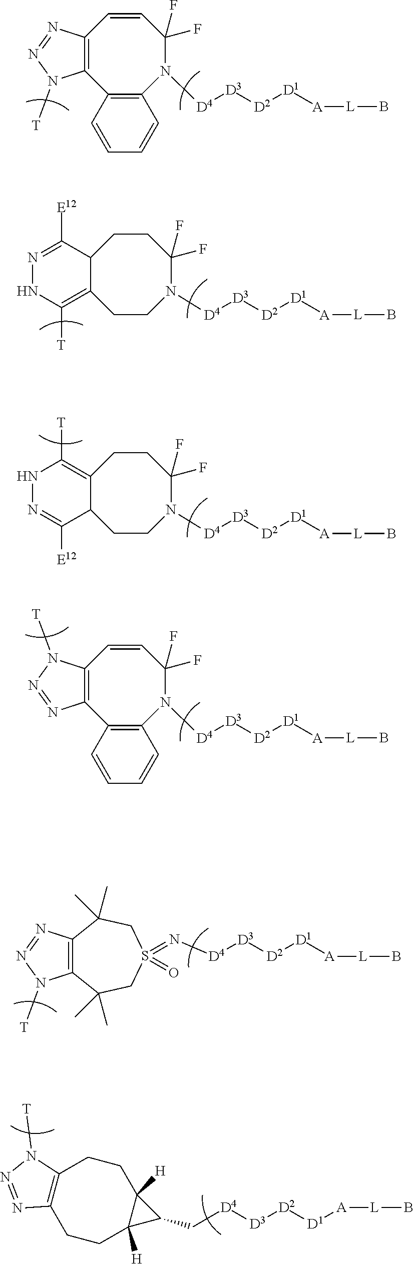 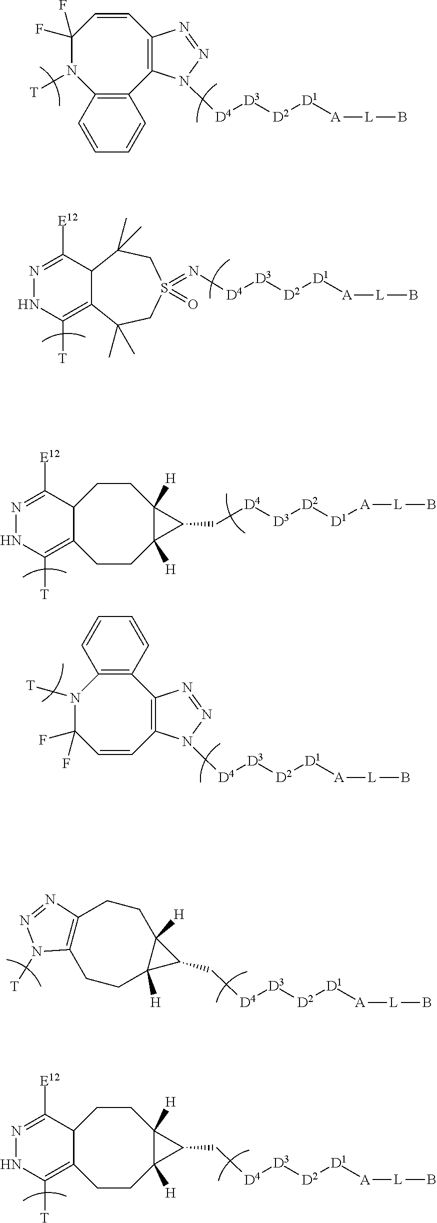 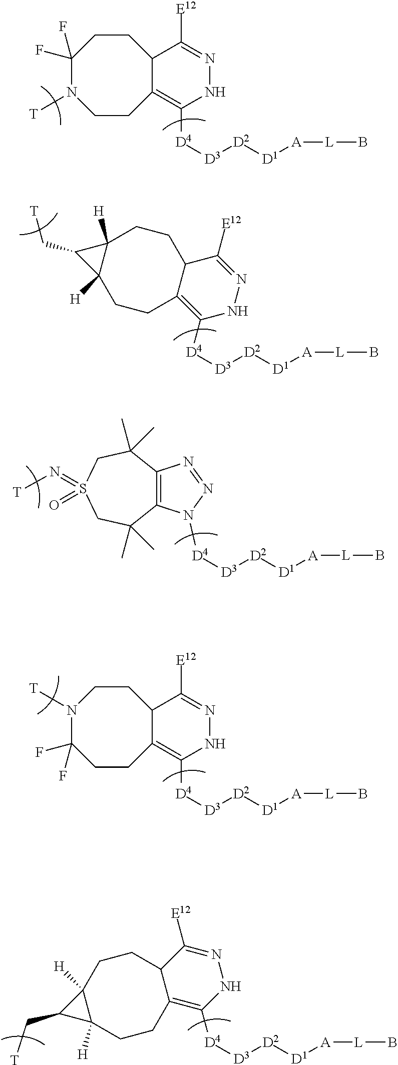 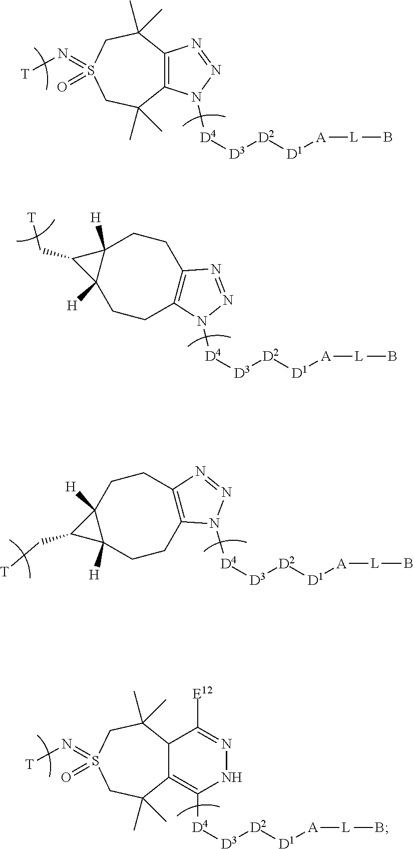
E¹ is selected from —H, —C₁₋₄-alkyl, —C(O)-Me, —C(O)—O—C₁₋₄-alkyl, —C(O)—NH—C₁₋₄-alkyl, and —S(O)₂—C₁₋₄-alkyl;
E² is selected from —H, —C₁₋₄-alkyl, —C₁₋₄-alkyl-SO₃H, —C₁₋₄-alkyl-OPO₃H₂; —C(O)-Me, —C(O)—O—C₁₋₄-alkyl, —C(O)—NH—C₁₋₄-alkyl, —S(O)₂—C₁₋₄-alkyl, —CH₂—CH₂—O)₁₋₈₀—CH₂—CH₂—OH, and —CH₂—CH₂—N(CH₂—CH₂—OH)₂;
E⁶ is selected from —O—, —NH—, and —S—;
E⁷ is selected from an aromatic or heteroaromatic residue, preferably -Ph;
E⁹ is selected from —O—, —NH—, —N(C₁₋₄-alkyl)-, —CH₂—, and -1,2,3-triazolyl-;
E¹¹ is selected from —H, —C₁₋₄-alkyl, —NH₂, —NH—C₁₋₄-alkyl, —NH(C₁₋₄-alkyl)₂, and —O—C₁₋₄-alkyl;
E¹² is selected from —H, -Me, -Ph, -2-pyridinyl, and -5-pyrimidinyl;
G¹ is selected from —NH—, —O—, —S—, and —Se—;
G² is selected from —CH═C(E¹¹)—, and —CH₂—CH(E¹¹)-;
G³ is selected from —NH—, —O—, —S—, —Se—, —CH═C(E¹¹)—, and —CH₂—CH(E¹¹)-;
Z¹, Z², Z³ are independently selected from CH, and N; and
T, A, L and B are as defined claim 1.

10. The compound according to claim 1, wherein
the compound having the formula (I) is selected from 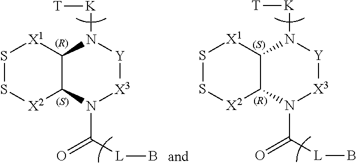
and mixtures thereof;
wherein
X¹, X², X³, Y, T, K, L and B are as defined claim 1.

11. The compound according to claim 1, wherein
the therapeutic agent A¹-OH, A²-NH-A³, A⁴-SH, or A⁵-C(O) OH is selected from a therapeutically acceptable cytotoxic, cytostatic, or immunosuppressive agent, preferably wherein the therapeutic agent A¹-OH, A²-NH-A³, A⁴-SH, or A⁵-C(O) OH) is selected from DNA-alkylating agents, DNA-intercalating agents, DNA replication inhibitors, tubulin-inhibiting antimitotics, bifunctional degraders, antibiotics, immunomodulators, kinase inhibitors.

12. The compound according to claim 1, wherein
the therapeutic agent A¹-OH, A²-NH-A³, A⁴-SH, or A⁵-C(O) OH) is selected from topoisomerase inhibitors, duocarmycins, pyrrolobenzodiazepines, trioxacarcins, nitrogen mustards, calicheamycins, mitomycins, doxorubicin derivatives, toxoids, auristatins, tubulysins, folic acid analogues, nitrosoureas, pyrimidines, tamoxifens, androgens, maytansinoids, aziridines, methylamelamines, platinum complexes, bifunctional PROTAC degraders, and molecular glue degraders.

13. A pharmaceutical composition comprising the compound according to claim 1, or a stereoisomer, racemic mixture, pharmaceutically acceptable salt, ester, hydrate, or solvate thereof, and a pharmaceutically acceptable carrier or excipient.

14. A method of treating, ameliorating, preventing a disorder selected from a neoplastic disorder; atherosclerosis; an autoimmune disorder; an inflammatory disease; a chronic inflammatory autoimmune disease; ischaemia; and reperfusion injury, wherein a therapeutically effective amount of a compound according to claim 1, or a stereoisomer, racemic mixture, pharmaceutically acceptable salt, ester, hydrate, or solvate thereof, is administered to a patient in need thereof.

15. A method according to claim 14, wherein the neoplastic disorder is cancer which is preferably is selected from acoustic neuroma, adenocarcinoma, angiosarcoma, basal cell carcinoma, bile duct carcinoma, bladder carcinoma, breast cancer, bronchogenic carcinoma, cervical cancer, chondrosarcoma, chordoma, choriocarcinoma, craniopharyngioma, cystadenocarcinoma, embryonal carcinoma, endotheliosarcoma, ependymoma, epithelial carcinoma, Ewing's tumor, fibrosarcoma, hemangioblastoma, leiomyosarcoma, liposarcoma, Merkel cell carcinoma, melanoma, mesothelioma, myelodysplastic syndrome, myxosarcoma, oligodendroglioma, osteogenic sarcoma, ovarian cancer, pancreatic cancer, papillary adenocarcinomas, papillary carcinoma, pinealoma, prostate cancer, renal cell carcinoma, retinoblastoma, rhabdomyosarcoma, sebaceous gland carcinoma, seminoma, squamous cell carcinoma, sweat gland carcinoma, synovioma, testicular tumor, Wilms' tumor, adrenocortical carcinoma, urothelial carcinoma, gallbladder cancer, parathyroid cancer, Kaposi sarcoma, colon carcinoma, gastrointestinal stromal tumor, anal cancer, rectal cancer, small intestine cancer, brain tumor, glioma, glioblastoma, astrocytoma, neuroblastoma, medullary carcinoma, medulloblastoma, meningioma, leukemias including acute myeloid leukemia, multiple myeloma, acute lymphoblastic leukemia, liver cancer including hepatoma and hepatocellular carcinoma, lung carcinoma, non-small-cell lung cancer, small cell lung carcinoma, lymphangioendotheliosarcoma, lymphangiosarcoma, primary CNS lymphoma, non-Hodgkin lymphoma, and classical Hodgkin's lymphoma, preferably colon cancer, rectal cancer, small intestine cancer, brain tumor, leukemia, liver cancer, lung cancer, lymphoma, basal cell carcinoma, breast cancer, cervical cancer, melanoma, ovarian cancer, pancreatic cancer, and squamous cell carcinoma.

16. A compound having the formula (II)
K′-A-L-B (II)
wherein
K′ is represented by W′-D⁴-D³-D³-D¹-;
W′ is a functional group suitable for binding to an amino acid side chain in the targeting unit T; and
A, L, B, D¹, D², D³, and D⁴ are as defined in claim 1;
or a stereoisomer, racemic mixture, pharmaceutically acceptable salt, ester, hydrate, or solvate thereof.

17. The compound according to claim 16, wherein the compound having the formula (II) is selected from 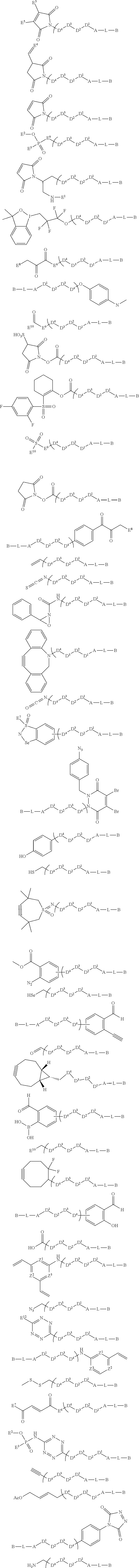
wherein
E¹ is selected from —H, —C₁₋₄-alkyl, —C(O)-Me, —C(O)—O—C₁₋₄-alkyl, —C(O)—NH—C₁₋₄-alkyl, and —S(O)₂—C₁₋₄-alkyl;
E² is selected from —H, —C₁₋₄-alkyl, —C₁₋₄-alkyl-SOSH, —C₁₋₄-alkyl-OPO₃H₂—C(O)-Me, —C(O)—O—C₁₋₄-alkyl, —C(O)—NH—C₁₋₄-alkyl, —S(O)₂—C₁₋₄-alkyl, —(CH—CH₂—O)₁₋₈₀—CH₂—CH₂—OH, and —CH—CH₂—N(CH₂—CH₂—OH)₂;
E³ is selected from —C≡C-E¹¹, and —CH—CH-(E¹¹);
E⁴ is selected from —H, —C₁₋₄-alkyl, and —O—C₁₋₄-alkyl;
E⁵ is selected from —F, —Cl, —Br, —I, —OMs, and —OTs;
E⁶ is selected from —O—, —NH—, and —S—;
E⁷ is selected from an aromatic or heteroaromatic residue, preferably -Ph;
E⁸ is selected from —F, —Cl, —Br, —I, —OMs, —OTs, —OTf, —C₁₋₄-alkyl, —H, —O—C₁₋₄-alkyl, —(CH₂—CH₂—O)₁₋₈₀—CH₂—CH₂—OH, —CH₂—CH₂—N(CH₂—CH₂—OH)₂;
E⁹ is selected from —O—, —NH—, —N(C₁₋₄-alkyl)-, —CH₂—, and -1,2,3-triazolyl-;
E¹⁰ is selected from —F, —Cl, —Br, —I, —OMs, —OTs, —C≡C-E¹¹, and —CH═CH-(E¹¹);
E¹¹ is selected from —H, —C₁₋₄-alkyl, —NH₂, —NH—C₁₋₄-alkyl, —NH(C₁₋₄-alkyl)₂, —O—C₁₋₄-alkyl;
E¹² is selected from —H, -Me, -Ph, -2-pyridinyl, and -5-pyrimidinyl;
Z¹, Z², Z³ are independently selected from CH, and N; and
A, L, B, D¹, D², D³, and D⁴ are as defined in claim 16.

18. A compound having the formula (III)
K′-A-L′ (III)
wherein
K′ is represented by W′-D⁴-D³-D²-D¹-;
W′ is a functional group suitable for binding to an amino acid side chain in the targeting unit T;
L′ is a leaving group or an activated self-immolative spacer unit; and
A, D¹, D², D³, and D⁴ are as defined in claim 1;
or a stereoisomer, racemic mixture, pharmaceutically acceptable salt, ester, hydrate, or solvate thereof.

19. A compound according to claim 18, wherein the compound having the formula (III) is selected from 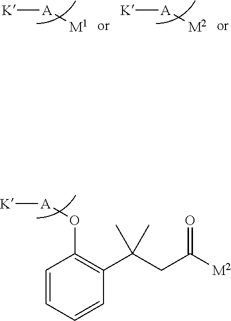
wherein
K′ and A are as defined in claim 18;
M¹ is selected from —F, —Cl, —Br, —I, —OMs, —OTs, —OTf, and —CN;
M² is selected from

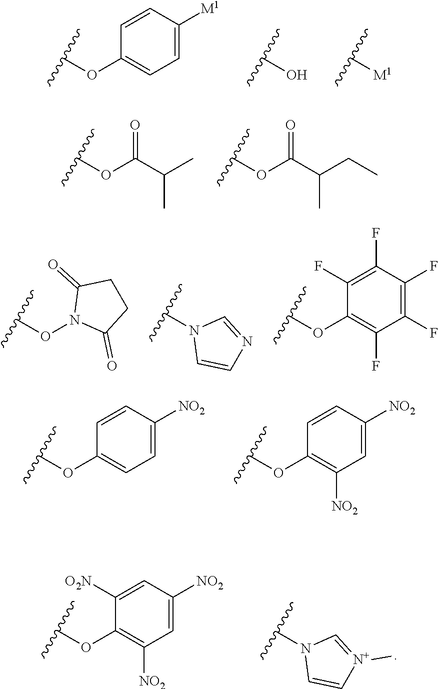

20. A compound according to claim 18, wherein the compound having the formula (III) is selected from 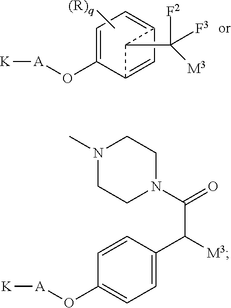
wherein
M³ is selected from 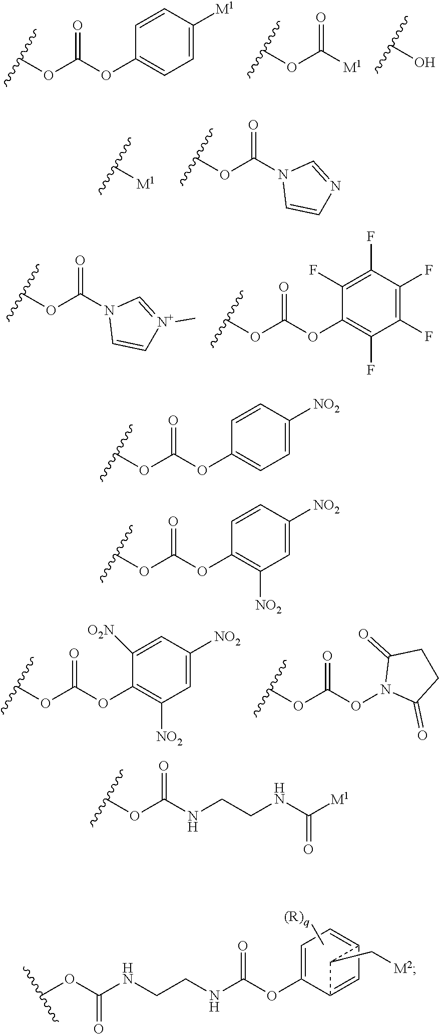
K′ and A are as defined in claim 18;
F² is selected from —H, —CF₃, —C₁₋₄-alkyl;
F³ is selected from —H, —CF₃, —C₁₋₄-alkyl;
M¹ is selected from —F, —Cl, —Br, —I, —OMs, —OTs, —OTf, and —CN;
M² is selected from 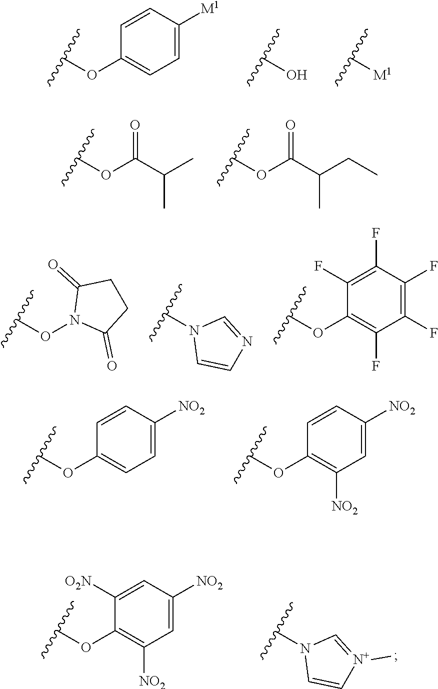
R is independently selected from halogen, —O(R^{r}), —N(R^{s})(R^{t}), —NO₂, —CN, and a heterocyclic group selected from azetidinyl, pyrrolidinyl, piperidinyl or morpholino, wherein the heterocyclic group is attached to the phenyl ring via the N atom;
R^{r} is selected from —H, —C₁₋₄-alkyl and —(C₂₋₄-alkylene)-O—(C₁₋₄-alkyl);
R^{s} is selected from —H, —C(O)—C₁₋₄-alkyl and —C₁₋₄-alkyl;
R^{t} is selected from —H, —C(O)—C₁₋₄-alkyl and —C₁₋₄-alkyl; and
q is 0, 1, 2, 3 or 4.

21. A compound having the formula (IV)
Q¹-D²-D¹-A-L-B (IV)
wherein
B, L, A, D¹ and D² are as defined in claim 1; and
Q¹ is selected from —CO₂H, —NHFmoc, —NHBoc, —NHCbz, —NHBn, —CCH, —CHCH₂, —CHO, —NH₂, —N₃, —OH, —Br, —I, —Cl, —OTf, —OTs, —OMs, —CO₂tBu, —OAc, —OTFA, —CO₂Bn, —CO₂Ac, —CO₂Piv, —CN, —SH, —SAC, —SMs, and —STs;
or a stereoisomer, racemic mixture, pharmaceutically acceptable salt, ester, hydrate, or solvate thereof.

22. A compound according to claim 21, wherein the compound having the formula (IV) is selected from 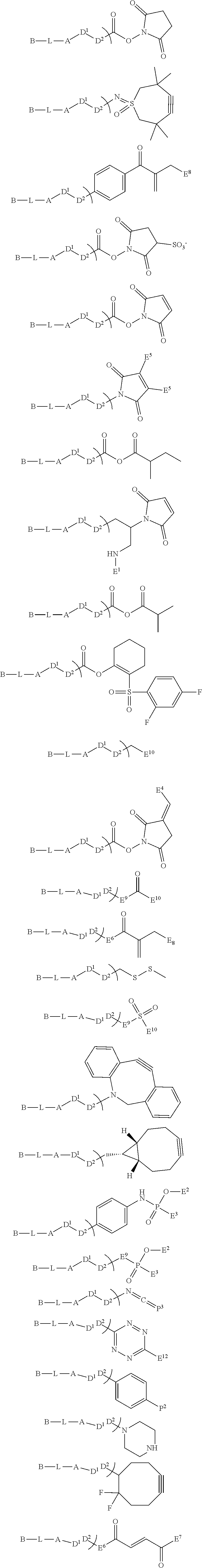
wherein
E¹ is selected from —H, —C₁₋₄-alkyl, —C(O)-Me, —C(O)—O—C₁₋₄-alkyl, —C(O)—NH—C₁₋₄-alkyl, and —S(O)₂—C₁₋₄-alkyl;
E² is selected from —H, —C₁₋₄-alkyl, —C₁₋₄-alkyl-SO₃H, —C₁₋₄-alkyl-OPO₃H₂; —C(O)-Me, —C(O)—O—C₁₋₄-alkyl, —C(O)—NH—C₁₋₄-alkyl, —S(O)₂—C₁₋₄-alkyl, —(CH₂—CH₂—O)₁₋₈₀—CH₂—CH₂—OH, and —CH₂—CH₂—N(CH₂—CH₂—OH)₂;
E³ is selected from —C≡C-E¹¹, and —CH═CH-(E¹¹);
E⁴ is selected from —H, —C₁₋₄-alkyl, and —O—C₁₋₄-alkyl;
E⁵ is selected from —F, —Cl, —Br, —I, —OMs, and —OTs;
E⁶ is selected from —O—, —NH—, and —S—;
E⁷ is selected from an aromatic or heteroaromatic residue, preferably -Ph;
E⁸ is selected from —F, —Cl, —Br, —I, —OMs, —OTs, —OTf, —C₁₋₄-alkyl, —H, —O—C₁₋₄-alkyl, —(CH₂—CH₂—O)₁₋₈₀—CH₂—CH₂—OH, and —CH₂—CH₂—N(CH₂—CH₂—OH)₂;
E⁹ is selected from —O—, —NH—, —N(C₁₋₄-alkyl)-, —CH₂—, and -1,2,3-triazolyl-;
E¹⁰ is selected from —F, —Cl, —Br, —I, —OMs, —OTs, —C≡C-E¹¹, and —CH═CH-(E¹¹);
E¹¹ is selected from —H, —C₁₋₄-alkyl, —NH₂, —NH—C₁₋₄-alkyl, —NH(C₁₋₄-alkyl)₂, and —O—C₁₋₄-alkyl;
E¹² is selected from —H, -Me, -Ph, -2-pyridinyl, and -5-pyrimidinyl;
P² is selected from —OH, —NH₂, —NH(E²), —SH, and —SeH;
P³ is selected from ═O, ═NH, ═N(E²), ═S, ═Se; and
B, L, A, D¹, D² are as defined in claim 21.

23. A compound with the formula (V)
K″-A-L′ (V)
wherein
K″ is represented by Q¹-D²-D¹-,
Q¹ is selected from —CO₂H, —NHFmoc, —NHBoc, —NHCbz, —NHBn, —CCH, —CHCH₂, —CHO, —NH₂, —N₃, —OH, —Br, —I, —Cl, —OTf, —OTs, —OMs, —CO₂tBu, —OAc, —OTFA, —CO₂Bn, —CO₂Ac, —CO₂Piv, —CN, —SH, —SAC, —SMs, and —STs;
L′ is a leaving group or an activated self-immolative spacer unit, and
A, D¹, and D² are as defined in claim 1;
or a stereoisomer, racemic mixture, pharmaceutically acceptable salt, ester, hydrate, or solvate thereof.
